SEQUENCE LISTING (28 sequences)

SEQ ID NO: 1 - DNA, 33 bp, Artificial Sequence
  Primer
ggtctagaggtcagtgcccccgcgtaccgtcgt

SEQ ID NO: 2 - DNA, 26 bp, Artificial Sequence
  Primer
ggcatatgcttgtgctcgggctcaac

SEQ ID NO: 3 - DNA, 36 bp, Artificial Sequence
  primer
cccgcccgcgcgagcggcgcgtggccgcccgagggc

SEQ ID NO: 4 - DNA, 36 bp, Artificial Sequence
  Primer
gcgtcctcgcgcagccacgccaccagcagctccagc

SEQ ID NO: 5 - DNA, 35 bp, Artificial Sequence
  Primer
ccaaccccgccgcgtccccggccgcgccgaacacg

SEQ ID NO: 6 - DNA, 34 bp, Artificial Sequence
  Primer
gtcgtcggctacgggccggtggggcagctgctgt

SEQ ID NO: 7 - DNA, 35 bp, Artificial Sequence
  Primer
gtcggtggactgccctgcgcctgatcgccctgcgc

SEQ ID NO: 8 - DNA, 35 bp, Artificial Sequence
  Primer
ggccggtggtgctgcccgaggacggggagctgcgg

SEQ ID NO: 9 - DNA, 39 bp, Artificial Sequence
  Primer
caccgctcgcgggggtggcgcggcgcacgacgtggctgc

SEQ ID NO: 10 - DNA, 38 bp, Artificial Sequence
  Primer
cctcctcggacagcgcgatcagcgccgcgcacagcgag

SEQ ID NO: 11 - DNA, 100588 bp, Actinosynnema pretiosum
gatctggggcgacgagccgcccgccgggccggggccggcgttgcaggcgctcgtctcccggctgcggcgggcgctcggcgcgccgggcgcggtcgcgctgggggtgggcgggtaccggctcgtggcggacgtggacgcggcgcggttcgaggagctggccgcgcggggcggggaggacgcgctgcgggaggccgccgcgctgtggggcgggcgggtcgggggcgagccgccggtggtcgcggccgtcgcgccgcgggtggcgacccggctggcgcggctgtcggtggaggtggtgctggacctggcggaggtcgagctggcgctcgggcgcaccggggcggccatcggtggggcgagcggggtgctggccgagcacccggcgcacgagcgggccgccggggtgctggtggacgcgctcgcgggcgcgggacggcaggccgaggcgctggcggcctacgagcgggtccgcgcggcgctggccgacgagctgggcgccgaccccggcacggccctgcgcgagcgccacctgcggctgctgcgcgccaccccgccaccgctcccccggccgaacgcgctgcccgcgccggtgacgggcttcctcggccgggacgccgacctcgcccgcgtcgccgacctgctggccgccgggcggctggtcaccgtcgtcgggcccggcggggtgggcaagacccggctggccgtggaggcgctgcgccgggaccgggacgcgctgctggtggacctcgcgccggtcgccgagccctcggaggtcgtcgccgccgtgctcgccgggatcgggctgcgcggcgaccgcgaccggccgggcggggacgcgacggcgctgctggccgccgagctggcggcgcgcaggtcggtgctgctgctggacaactgcgagcacctggtcgacgccgtggcccacctggtcgcgctcctgctcccccgctgccccgagctgcgcgtgctcgccaccagccgggaacccctggcggtcgacggggaggcgctggtcccgctggggccgctcgcgctgcccggaatcggggacgggcttgacgccgcggtcggcacggcctcggtgcggttgttcgcccaacgggcgtcggcggtgcgccccggtttcgccgtcgacgccacgacgctgccggacgtggtgcgcctggtgcgggcgctggacgggctgccgctggcgctggagctggccgccgcccggttgcgcgccctgccgctgcccgacctggtggccgggttgtcggcgcggttccgcctgctggcgggcgggaaccgggccgcgccgccccggcaccgcacgctgcgcgcggtgatcgcgtggagctgggacctgctggacgggcccgagcgggccgtggccgagcggatctccgtgctgcccggcggggtcaccccggagtcggccgccgccgtctgcgcgggcgccgtgcccgccgacgaggtgcccgaactgctggccgcgctggtcgaccggtcgctgctgagcctggtcgggggtcggcggcggatgctggagacggtgcgcgcgtacggggtcgagcgcctggccgccgccggggacttgagcgcggtccgcgacctggccgccgcgcacgtggcgggggtgctggcggggcaggacgcggtgctgcgcgggccggggcagcgcgcggcggtggcggcgatcggcgcggagcacgacaacgcggtggccgcgctgcaccaccggtgcgccaccggggacgcggacggggcgctcgcgctggcgctgtcgctggtctggtactggcaggtgttcggccgccagtccgagggcgcgcactggctcgggcgggcgctggcggtgcccggcgggccgtccccggagcgggactgcgcgcgggccgcccacctgctcggcctggccgacggcgggcacggggtgggtgatcgcggggaggtgggggcgctcgcggaccgggtgctggcgcaccgggggctccccggtcacctgcgggtgctcggggcggtcctgctgttcctgctggggcgcggcgagggggtgttccgggagctgggcgcgggcggcgggtggttgtccgggctggcgcacctgttcctggccgagctggcggagaacgcgggcgagctggaccgggcgcgcgggcacgcggaggtgtccctggaccggttccgggcggccggggacgggtggggcgtggcgggggtgctgccggtgcgggcgcgggcgcggcggtacgacgacctggacgggacgtgggcggaccttcgggaggcgcgggcgctggagggggagttcggggcgctgagccccggtgaccgggtgcgggcggacctgcggtgggtcgacctgcacgagcggcgcggtgacagcggggcggcgctggaggtgctggccgcggcccgtgctcggggggagcaggtcgcggtggtggacgcgcgggaggccgcgctgcgggtgcggctcggggacctggggcgggcgggtgagctgctggccggggtgggtggggcggtgggcgacctggcgcgggccgcgtatcgggtggcctcgggggacctggcgggtgcggagcgggcgttgcggcgggcgcgggtggtggcggctgcgagcggggagctgcccgcgctggccccggtggcggtgggggcggcggcgctggagcaggcgcgggggcggtgggcggggtcgggggtgctgctcgggacggccgcgcgggtgcggggcgcgcacgaccgcaccgaccccctggtgcgcgagctggtcgaccgggggcgggcggcggtgggcgggagcgcgttcgcggcggcgtacgcgcgggggtgggaggcggagcgggacgtggcggcggcgttcgtgctctgagcgccgggatcgggcgggcggggtcaggcgggcggggtcatgtgggcggggtcaggcgggccaggtcacacgtccagggaccccgcccagtccgcgatcgtccggacttcggcctgcgtcgggaagaccttctcggtgagcacgcggtgcacctcggggtcgccgtccaggcagccgtcggccaggacggtgagctggaagtccaggtcggcggcctggcggagggtggacaggaccacgccgctggtcgcgatgccggtgagcaccaggtggtcgacgccctgggcgcgcaggacgaggtccaggtcgctgcccgcgaacgcgctgacgcggcgcttggtcaccaccacctcgtcgtcgagcggcgcggtctcggggtggaagtcggtggcgccggagcccctgggggccgcggccaggcggccgaacatcttgttgcgcgggtggatctccgcgtagtcggggcggaagccgacgccgacgtggatcaccggcacggacgcggcgcgggccgcctcgagcgcggtggcgagcctggggaggtaggccgggtcggggtagcgggcgaccacggcgggctggacgtccatcaccagcagggcgggggtggggatctcgggcctcgtttcggtggtggcggcgcgggggccgccggtgggggtcaggggtgcgggggtgccggggtgagcaggctggtgacggtgagcaggcggtcggcgagttcctcggggcgcagcgggtcctcggcgcgcacgacccagtcgtggacgatcgcgccggtgccgtgggagatcagcacggccaggtcgcgggcggcccgctcgtccacctcgcccaggccggcgcgcagggcctcggtggtgatgagggtgcggaagagctcggccagggcctccgccagccgccacgcgcacgggccggtgagcacggcgcggtagaaggggcggtggtcggcgaagtggcgggccacggccaggaggcgggcgtggcgcggggcccgcgggtcggccaggtgcggcaggagctcgcgccgcaccaggtccgccgcagcggcgacgaggagcgtgtcgcggtcgccgaagtgctggtagagcagctgcctgctgacgtcggcggcctcggccaggtcggtcaccgggaccgccgccccgcgctcggcgaccaggtcgacggcggcggccatgagggcggccctggagcgggcgacccggcggtcggggcgggtggtcacgggggtgaaactagacagttgtcaataaatgagcaagtgtcgtcgaacgcgcgcgcgggaatctccggtgcgcggggcccgtccctggcagcatgatcacgcgatgaccgaggtgaggacgcgcccgtacgccgggcccgcggacctgcgcgcgatgcaggggttggcgcggcggatctggacgccgtcgagccggtggcacgtcggcgacctggcctggcagcgcaaccagcacaccgggcgcgaggccgagtggccgaccgcgctgtgggaggcgggcggcgaggtggtggcgtgggggtgggccgagctgccgggtgagctggcgctgctggtcgaccccgcccggccggagcttgcgggggcggtgctcgactggttcgcgggcgtggccaccgcgccccggcggtcggtcaccgtgctggacgccgaaccgcacctggtcgccgcgctggaggctcgcgggtacgagcggctgggcgggccgcacttccggcactcggtgcgcgcgctggacgacctgccgacgcccgaactgcccgccgggtaccgggtccgcgccgtgcggggcgaggaggacgtggcggcgcgggtcgcggcgcaccgggcggcctggtggccgtcgcgggtcaccgaggagagctaccgggcggtgatgggggcgtggccgtaccggccggggctggactgggtggtggaggggccggacgggcggttcgcggccacctgcctgatctggttcgacgagcgcaacggcgtgggcgagctggaaccggtcggggtcgaccccggtctgcggcggcgcgggctggggcgggcggtgtgcctggcggcgctgggcgcgctgcgcgaggcgggcgggcgggcggcggtggtgtacccgctgcacgggcaccccgaccaccccgcgcccgcgccgctgtaccgggggctggggttccgcgagcacgcccgcacgatcaccttcaccgcgctggaggcgcgcgggtagcagcggccgggcggggcgagcggacccggtcgacgagcggctccgctgtcggagcggtcgtcccagcgcgtggacaccagtgccacgaccagaccgcgccccgcttcgcgtggtcggctcgggggtcgaccgcggtgaggctctcgccggggtgggtgaaccacgtcctggcgatggcctgcaccgcgagcaccgggtgccgcccgtggcgctggacgtcaccgacgcagccgccgtcgaccgggccgggccggccgcgttcggccgttgcgccgcgccggccgagtccgacgccaggtggcggccggtccccgggtccgcctggaactgaccccgccggcctccccgcccgcccgtccggccgggcgccgaacccgcctcaggcgtgctcgaccgcgcgcaccgatccccccaccaccaccggcatcgggacgtggtgcacggtcgtcgggctgcggtcgcggcgggggcgggacaggaggagttccacggccatcgcgcccaggcggtggtgcggcagggcgacggtggtcaggcgcgggcgcatccaggcggccacggggtggtcgtcgaagccgaccacggagacgtcgtccggcacggacaggcccgcctccgcgagcgcctggcacgcgccgaacgccaggcggtcgttgaagcacagcagcgcgcgagggcggtggtgggacaggaggtccagggtggcgcggtagccgttctccggcatccactccacgcacgggcgcacgctctccacctccacccccgccgccgcgaaggtctccagcgcgccggagaggcgggccacggcggcgatgtggcgcgggtcgatcgcctcggccgtgggcccggtgccgatcaggtgcacgccctcgcggtgcccggcgtcgagcagcacgcgcgccgccgaacggccgccgccgcggtcgtcggggagcacggcgtgcgcggggaagtcgttggcgggcagcacgttcagcagcacggacggcccgtcgcgcagcccgtccgggacctccagcagccgggggaacctggccgcgaagaccacgccctccacctggcgggcgcgcagcgaggccaccagcgccgcctccacctcgcggtcgccgccgctctcaccggcgaacagggtgaacccgtgccggtgggcggcgccgaccgcgccctcgatcagctcaccggacagcttggccgaggccacggcgtccgagacgaaaccgagggtcttggtgcgggaggcggacagcagcgtgtcgcggcggtagccgagctgctcggccgtcgcccgcaccttgcgctccaccgccgccgagatgcgcagctcccgagcgcggccggagagcaccagggaggcggtgggcaccgacacggcgcaggcggacgcgacgtcggccagcgtgacgcgcgtccgcccgctctcgcggacacctgctcgcgggggtgtgcccgtcacccgtgcctcccgtcaccggtcgcgcgacagccccgcgcgaggtcctaccccatcgtgcaggccgcgccgttcaaggagaaccccgaaggtggggccgcgtccccgccgtgggtgacctggtagccgatgctgactttgccaccgggtgggatcgccgcgttgtagcccgcgtcgcgggcggtcacccggcccgagctgggcgcgtacgaggcgttccagccggaggtgatcacctggcccgcgggcagcgcgaactccagcgaccagccctgcacctgcgtggtcccggtgttggtgatggcgagctccgccgtcaggccgttgccccaggcgttgacggtggccgacacccggcaggcccccggctgcggttcgccgggcgtggtggtggtggtggtggtggtggtcgtggtcgtggtggtgctggtcgggtcggggccggttccggcgaactgggtgaagaaccgccaggtctcctcgggcgcccacgtcctggtgccgctgtcgccgggcgcgttgtcctgcggtgcggcgatgtggccctcgtcgaacgcgacccagcgcaccgggtagccgtcgcggcagccggtgtaggtggtgccccggtgggtcaggctgccctgggacggttccggcgggttctgcgcggcgcagccgttgttgcgcacgaaccggtcgcgcatcgagcgcccgccggagatgttcaggacgctgtcgcgcaggccgtggatgccgaggtaggcgatgggctgcgtgccgccggcgcagccgctgagcacgccgcccgcgatgaccgcgaccgcgcggaacaccgtcggccgcgagcaggccaccgagtaggacatcgcgccgccgtagctgaagccggtggcgaaccgctgggtggtgtccacgcacagcccggcgtcgagctggcggacgatgtcgtcgacgagggtgatgtcctcgccgccgttgttggcccagccgttgttgaagccctgcggcgccacgaagatcgtgctgctgcccgccaggcgcttgaggccgtagtaggaccagacgtcccgctgcacggtctggccggtggcgacgtcgttcgcggtgccgctgagccagtggaagccgaagacgacgcggtgggggcggttccggtcgtagccgtccgggatcgacaggatgtaggtgcgggacttgccgctgctggtgatcgtgcgcgtgccgctggtgagcgcgggcgccttgccgcagccctccgtcgtggcggacgcgccgggggcgccggacgcgccgggcgctccggtcgcgctggtggtgatcagccccgcggcgagggtgagcagcgcgatgcccgctgccgcgaggaccctgttgcgcgccaagggattcgcccttcctgtggtggttccggtgggtgtggtcacggggtggtgaggtcgaagcggcgggcggtgacggagccgccgagcgcggcggtggcgtggttgaagacggcgaagcggtagcccatgaagaaccgccagtcgttcttgagcgtgaacgccgggccgaaggcggtgaagttgacgccgtcggtgctgtaggagaaccgggcctgcctgccgttgccggggcggatgtcggcgttggcgcgcaaccagatccgggagccgcccaggtcggcgctcgcgacctcgtagccggttccggtggtgcgccaggagccgtccatggtcaggccggtgacggagacgatccggttgcggccgttgtcgcgcttgacgccgatccacgccgaggagtcgcgcagcacggccagcccggtgcggtcgccgtcgcgcatccccgacaggtccagttccacggtgccggtggaggtggggccctggatgcggtgggtgagggtgttgcgggcggagtacaggtcgttggtgacggtcgcggtggacaggcgaaggccgttgttcacgctgtacttggcggtgtccgggttgtggttccactcccactgcgggccgagcgcggcgccggagaaggtgtcggcgccgatcatgggtttgacctggcgcgggggcgcgggcaggttcggcttcgggtaggtcgcgccccagccgccgttgacggtggtgacgcgcggccagccgtccgaggtccaggtgatcggggcgagcaccggcacgcgcccgccggggtaggcgtcgacgaacgccaggtagtgccagtcgccgttctgggtctgcaccaggccgccctggtgcggcactcccccgccctggatcggcgagggcaggtcgagcagcacctgctggatcgagtacgggccgaacgggctggacgacttgagcacgtactggccgttcgcgggcctggtgagccagatgtagtagttgccgccgcgcttgtagaagcgcgccccttcgagggtgccgatgttcgagggggtctggaacacctgctgggagcggacctccgacttcccgtcggcggagagctgggcgacgctgatgctggtgttgccgtaggcgacgtacagggtgtcgtcgtcgtccacgagcatcccggcgtcgtagtagcacttgttgatggtggtgtgcttggaccactggccgtcgacggcggtcgcggtgtacaggtgcgtctgggcgaagtcgacgcagccgccccagtagaaggtgcggttgctcttgcggtgcgccaggaacgacgcccagatgccgttgacgtacgcgcgggagccgttgcccatgtcgtacttggccccgaagtccaggcgtggcacggagtgcccggcgaactcccagttgaccaggtcgtaggagcgcagcacgggcgcgccgggcgagtagtgcatggtggaggccgagtagtagtaggtgtcgtccacgcgcaggacgtcgatgtcggcgaagtcctgccacagcacggggttggtgtaggtcccggccgcgcgggccgggtgggtggtggtggcggtcaggctcgccgccacgaggccgagcacggccagggcgatgggcgcccatcggcgttgacggggcatcggtgtgcctctcctggtgtccgggagttggctctgggcgcggcggcggtggacttgtcgggcgcggcggtggtcgtgggggtcagcagggggagttggtctgggtcagcaggcccagccgccagggcaggcggttgtagtcgccggacgcgttggggtccaggccctggtacaggtagctcagcttgcaggggttgatctccatggtctggtcggtcccgctgcgcaccagctcgccgtggctgatgtcgcgcgtccactggccaccggggaacgtggtgttgttggccctggcgaacgggttcgactcgctgtcggccagcgcggtccacggtccggcgatcgccggggcggtccaggagcggaaccagcggcggccgtccgagccgatcgcctcgtggagcatcagccactggttcttgccggcgaccttgtagatgttggacgcctcgaacaaccggttgcggttgctgtcctgcatggcgatcacggtgttggtgaagccgttggggaactgggcgaggctggtctccgagcggtacaggtggccgttgtcgtccgaggagaacaggtggcacttggccgtgtcgcagacggtccagaagtcgacccagtagccgttgccgatgttgtcccggatgatctgcggcatcccgttggcgtagaagttcctcggcgcggaccaggacgcggggttctcgatgtcggcggtcgtcgagtacgaggcgttggacccggtctggtacaccaggtaccacaggcgttgcggggcgaagtagaacacctgcggcgcggcccggtagcccgtgccgatcccggagcggtccaggtagtggtgcggggcggacgcggcctgggaccagtcggtgaagctggtgtgcacgaggttgtagccgttggtgtagaccgaggcgaacacgtggtagcggccgttgtggcgcaccacgctggggtccttgacggagaccgtggcgtgcgaggagtcgggcttgggtccgatcagcgcgccgctggaggaccaccggaagctgctcggcagcgagccgcccggttgctgcggggtcgtggtggtgggcggcgtggtcgggggcgtggtggtcgtgggcccgactcctcccgtgcacgtggtcccgttgaggctgaacgaggtcgggtcggggttggacccggtggaggtcgcggtgaacccgaactcgacgcggcccccggtggggatggcggcgttgtaggaggcgttgcgggcgctcacctggccgccggactgggacacctcggcgttccaggcctgcgcgacctgctggcccgagccgtaggtccaggtgagcgtccagccgtcgacggcgtcaccgaggttggtgatggcgacgctcgcggtgaaaccgccctgccactgggaggtcgccgcgtaggcgatcgagcaccccggcgccgcggcggcctggggtgggagggcggcgagcgcggccaccatggcgagcgaggtggtggccatggcgccgatccgggcccggcgcggggtgaacagccttgcgaggagcatggtcgcccttcgtcgtcgtcgacgggtggtcggcgcggccgaccgggagcggcgggagcgggctggtactcccccacttcctgcaatctagccaggtggcacagggtggtcaaagctaaaaagggcggacgcggtttagcttccagcgcaaaggtttcgcgcgttctttcggccggggggcaggtggatcggggcgggctcggggcgaggacggggctgggaatggggcgggggatggggcgggctcggggcgggggtcgcccggagcccgccacgggtcagaggcgcacgcggacgacggtgaacgggaggttcggctcggcgatctggtactggaagttgaccaccagcaggtcgtcgccgtcgaaggtcgcggtggacgggacgtccatgcccctgccgttgacccgccgcagcaccgtggcgcgggagtggtcctcgctcagccgcaggacgctgatctcgccctccgggtggaacaggctggtgacgctgtagaggtcgttgccgcgcaggagcagcccgtccgagccgatgtcgcccacgccgcccaggtcgatcggggtgacggcgccggtgcgggtgctgatgcggtggaacgcctgggagttggtgtcggcgagcagcacgtggcggccgtccggggtgaccacgaggccgttgccgttgatgccctcctcgtagcgcaccggggagtcggcgaggtccacgaacgtcctcagtggctggtcgacctcggggctcgccagctgggcggcggtgatccggtagaggacggggcggaacgagtcgctgacgtaggcgtcgccgttcggggcgatggcgacgtcgttgaccaggccgtcgcgggcgccggagtcgaacacgtgcaggagcgcgccggtgcgggtgctgtggacgaagaccttgccggtggcgccgcccgcgatgaccagcctgcctcgggtgatcttcatgccgacggcggtggtgcggccgtgctgaccggcgggcaggaacggctccagggcggggcggtcgacgtggccgcgccagatcgtgccgtcggtcgtgccgccgacgtagaagtgcggcgtgcccggctcgcggacgatgccctccgggtaggcgcggtcgccggggaccacgtagcgggtgacggggtggtgcgcggcggcggcgggtggcacggcggcggcgggtggcgcggcggcggcgggtggcgcggcggcggggagggctggggcgagcgcggtgaggagcagggtcgcggtgaggagggctcgggtggtggtcacggaagggctccgggggtcgaaggggtgtctggcgccagacaagcgcttcgtggcgcggggtggcagtgggcgcttgtcgggggtagttcttcaccccccttccgggcggggcggccgactagggtgagcggtgtgggcgatcttgggcggcacccggtggcgaaccgctccgacgtgcgggacttcctggtcagcaggcgcgcgagggtgagtccggggcgggccgggctgcccgtgctcgggcggcggcgggtgccggggttgcggcgggaggaggtcgcgctgctcgccggggtcagcgtggactggtacacccgcttggagaaggggcacatcggcggtgtctcgcgggaggtgctcgacgccgtggccggggtgctgcggctcgacgccgaggagcgggtctacctgttcgacctggcgcgcgcggcccggcgtccccgcgccgccgaggtggcggcggaggccgcgctgcccgcgacggcgcagtggctgctggacagcatgacgctgtcgtcggcgatggtgaccgggcggcggcaggacgtgctggcggtcaacccgctggcccgcgcgctctacgcgccgctgttcgccagcgccaccacgcgggacggcggccgggcgaacctcgcccgctaccacttcctcgacgcgggcgcccgcgagttctacggggactgggcgggcaccgccgacgtgctcgtcgccgcgctgcgcgccgaggccgggcgcgacccgcgcgacggggccacccgcgagctggtgggcgagctgacggccgcgagcaccgagttccgggcgcggtggagcgcgcacgacgtgctgctgcacccgcgcggcgccaagaccttccggcaccccgaggcgggtgagctgagcctgagctaccactcggtggacctgccgatctccgccaccgagacccggcacgtgtgcgcgtgcaccgccgaacccggctcgaccgacgaggcgaggctgcgcgcgctcgtcgggtgagccgggggtggccggccaccgccgtcgcgctcgcggcggcggggcggggccgccggtcagagcgtgagcgccatcccgatcgagccgggcgcggtctcggtgaagccgtgcttgaggtacagcgggcggccgggcgcgtcggccaggagggtcacgaacgcgccgggcggggcggcctcgcggatgcgccggagcagcgcgtccatgatcgcgccgccgacgccccttccctggtggtcgggcagcacggccatgtcgacgacgtggaagtaccagccgccgtcgccgaggacccggcccatgccgacggtcccgccgtccgcgtgcgtgacgtggaaggaggcccaggcgccgggcagggcggcggcggcctgctcggcggtcttgggcgacaggccggactcggcgcgcaggcggaggtagtcggcgacggacggcggggtcgggtggagctcgtagtcccggtgcacgcggtcaggctcccacgggcgcgggcgggcccccgcccgacctgacgatttccccgtcggcggggatgcgggcgggcgctcgcggattttcgacatcccccggcccggcgagacgcggcggcgccgctgaaaagagcgccgtcgcggcccttcgcgccgcccccgacatcccccgcgcggcgaccggtcaatgcggtccacgcctgggggtttccctcccacgtcgaacaccgccaccacgcgcccacgcgccgcgtcgaccaccccgacgccgaggaacacctgttcacgggcacgggaagccgcagcggagggggaaccgggaatggccgcaggcgatcgcggcacgacgtccgcacatcaccgcgagcagaatcgcgaggcgttcaccggggcggcggaggaagattccagcgcccctctcgaagaacctgcgggaagccctggaagaaaacccggacccgaaacgcgacaaaattgcggacacccacccgtgaaacaccgggcgcccccaccaggtcacccgctgacatcacgctcagtcagtatcggcacgctcccccgccgaggcggagcgcgacaccccgcccaaccgggcaccgagcgggcacctccactcggcccagcaccgccccaagatcgcacgtagcacgggttgaaaccgctcaagcgcatctcaacccgttcggagcagagtggcgcccgtcacgtcccgaccggtcacggttggcaacgggtccagtccacgcgaggtggcatcaagcgcacttgccccgatcacacccgcccgtgcaaccgaatgcagcagggatatctttcccgagaactcggccgttaaccgggagtggagccaggcccacccctaagacgcttgcccacatgcccaacaatggtgaagatggaacggcggaccgcaccgcgaacgcgaaccgaactccgcgagagggcacggtgaacgatcctggaacagctactgccgcgtagctcaagggtggaacgcccggctcgcgcgcggcggagggaataacggcttttacgccctcgacaacagcttgtcaacgaaacccgtgcacccgagcggtccccgcgcgcaccgctcgcgggggtggcgcggcgcacgacgtggctgcccggcgtcgacgacgacgcgagttccccgaccgcccgcgaaggcggtcgcggatcgccacgacggggcacccggaccacgcctcccccggaacagcgcgcccacgcgcggttccggcgcgcgccgggaccgccgcacccgccggagcgccgccaccggccggggccggtccccgcggaccgggtcgccttccggaccaccactccacggaccacggaaaggaccactcccccagtggagcttctgcgcgcacccgagatccagtcggccgtcgagcacctcgcggtggacctgccggaccgggcgggacgggcgttcctggtggacggaccgcccgcctgcggcaagacgacggccctgcggcggctcgtcgaccggatcgcccacgaggaccacctcgtgctcaccgccacctgcaccccgccggagacggagctgccgttcggggtgctcaagcagctcctcgcctcccccggcatggccagggtcgacccgcgcctggtcgccgacctcggcgagctgctcgccccggccccgccgcccgccgacgactcggcgctcctgcagctgtaccactcgctgtgcgcggcgctgatcgcgctgtccgaggaggtgccgctggtcatcgcggtggacgacgtccgccacggcgacaccgcctcgctgcacgtgctgctgcagctggtgcaccggctggacacggcgcgggtgcggctgctgctcaccgacgacctgctgctgccggtgagcttcccgccgctgcgctacgagctgctgcgcctgcgcgggctcggcctggtccgggtcgcgccgctgcctgcggccagggtgcgggaggaggcggtgcgggcggtcggcgcggacgtcgcgaagcgggtcgacttcgccgcgctgaccggcggcaacccgctgctgctgcacgcgctggcggtggacgtgctggaggcgggcgagccgcgcgagatcggctacggcaactcgttcctgtcctgcctgcaccgcaacgaacccctgttcctggacaccgtgcgggcgctggccgtgctgggcggcggctcggcgtcggacctgggcaggctgtccgggcacgagccggagcaggtcgcccaggtgctgaacgccctgcgggagtcggggctgctggccgaggacgggttccggcacgacgcggcgcgccgcgcggtcgtcgcggacaccccggtcgccgagcacgaggtgctgcaccgccgcgccgcgcggctgctgcgcgaccagggcggcgcggtcaccgacatcgccgaccacctgctgcgggcgggccgcatcaccgacccgtgggcggcggacctgctggtggacgcggcggagctggtggtgcagcgcggcgagccgacggcggcggtggcgctgctccagcgcgcgctcgactgcagcccggaccgggagcgcaggacggccgtgcaggcgcggctggccacggccgagtggctggtgaacccgtcgacctcggcaaggcaccacaccgcgctgctggcggcgttccacgcgggcaggttgtcggtgcgcgacagcgcgacgctgatgaagcacctgcgctgggccgggaacaccgccgactcggacgcggtgctcgcccggctgcggaccgacccgcgcgccgccgaggacgtgccggtgctggagcactggctgaccagcacctaccccggcgcggcccggcccaggaccgtgctggggcgggacgtggactcggcgcgcagcagggcggacctggtgccgagggcgaacgcggtgctgctggacgtgctggtggccggggacagcgacgacgtggccgaccgggcggaggcggtgctgcgggagctgcggctggcgcgggagtccgggtgctacggcggtgcggccgtgctggcgctgtccgcgctgctctactcggaccgcgcggacgtggccgcctcgtggtgcgagcagctgctgtcggcgcgggccgtgccgctgctgccgatgccgcgcgcgcaggtgctggcgctggcggccgagtcggcgctgcgccggggcgaccacccgagcgcggacgagctggcgcgggaggcgctgaccgtggtgtccccgaccgcgtggggggtgtcggtggggctgccgctgagcaccagggtgctggcgctgaccaggatgggccgctacgacgaggcggcggccgtggtggcgcagccggtgccgaacgggatgttcgggcaccgcaacagcgtggactacctgtacgcgcgcgggcacttcttcctggcgcgggaacggccgcgcgcggcgctgggcgacttcctgctgtgcggggagcagctgacccggtggggcctgggcagcgggtgcgcgccggtgccgtggcggaccgcggcggcggaggcgtggctggcgcagggcaaccgggaccaggcgcgggtgctgatccacgagcagctcggcaggccgggcacggacagccgccgggcgcgcgggcaggcgctgcggctgctcgcggcgaccagctcggtgaagcggcacccgcagctgctgcgggaggcggtggcggtgttcgaggccgtcgacgacaagtacgagctggcgcggaccctgcgcgacctggggagggcgcagcgggcgctgggcgagaacaagctggcgcgccgggtgatccggcgggggtggcacgtcgcccggatgtgcgaggcggcgccgctgtgcgaggagctgatgcccaccgccgacgggctggtgcccgcgcagcccgcgtcggcggcccgcaggtcggacctggaccggttgaccagctcggagcaccgggtggccgcgctcgcggcgtcggggctgacgaaccgggagatcgcggtgaagctgtacgtcacgcacagcacggtggagcagcacctgacgcgggtgttccgcaagctcgggatcaagcagcgggagcagctgccgccggagctgagcgtcgaccggtcgaagtgacgcggacggggcggtcccgtggatctggggccgccccgtccggtcccggtccgtccggtcccgccctgtccggtcccgccctgtccggtcccgccccgtccggtcccggtccgcctcaggctcggggcatcgcggccagggtggtggcgacgacgtgctcgtcgacgtcgcgcaccagctcggcgccgcgcgggccgtcgagcacgaacgccaggccgccggtggacttcttgtcgcggcgcatgaaccgcagcagctcgtcgtccggcacgcccggcggcagcgcgacgggcagcccgtagcccgcgaccacggagtggtgctcggccacccggtccgggccgatccggccgagcgcgcccgcgaggcggccggcgaagaccgtgccgatcgcgacgccctcgccgtgccgcaccgcgaaaccggtggcgagctccagggcgtggccgagggtgtggccgtagttgagggtgtgccgcaggccggagtcgcgctcgtcggcggccacgacgcgggccttgagggcgacgctcgccgccacctggtccagcagcggcagccggtccaggcccggcgcgccgatgaagtggcagcgggcgatctcgccgaggccgttgcgcagctcgcgctcgggcagggtggcgagcaggtcgaggtcgcacagcacggcggcgggctgccagtaggcgccgacgaggttcttgccctcggggaggttgaccgccgtcttgccgccgacgctcgcgtcgacctgggccagcagcgaggtcggcacgtgcaccaccggggtgccccggtggtagagcgaggcggcgaggccgaccgcgtcggtggtggtgccgccgccgcaggagacgacgacgtcggcgcgggtcaggccgaactcggcgaaccggctgcacaggtgggcgacggtggcgagggtcttgtcgtgctcgccgtcgcgggccgggaggacgagggaggggacgccggggtcgggcgtctggtccgccgggcgggcggtgaccacgacggcgcggcgcgcgccgagggcccgcacgacgtccgggagggcggcgcgcacgccgtgtccgatgtggacggtgtaggcgcgctcgcccagctcgacccggacctcgcgggtggtggcggcggtgggggcggggtgggtggagctgggcactgcttcctcctcgggtgggcgggacggggggcgatcgggggacgcggaggggtgacgggaaagcaatcgggcaggaatgggaacgggtccgggggcgaacgggcaggaattcgaatgggggcaagcgaccgggagcgatcccagtggtggggcggaagtgcgggcggcggaaaggcggtcgtgctgcctcagccgccgcccgcggcgcccgtcacgagcgtggtgcgcagggtgagcgccgcccgcgccgccacggccgcgccgacgccgaggcggttgtgggtcagggtcgcctgctcgaacagcaccggcagggccgggcggcggcggtccgcggcggcgcgcagctgctccaggtagcgccgccacgcgcccgccgagccgaccacgcgcccgccgggcggcgccccctgcgcggcgacggcggcctcggtgcgctcgaccgggcggagcggcctgctccagctctgccagcagtggaacaggaacgcgggcctggccggttccggggccagcgcgaccagttcggccaggtggtgcagcaccgtggcctgcgcgcccgactcgccagggtcctcgccccacaccgggctcaccaccgacgcgacgtccagggccagttcgctggacgcggtcgcgaccagcggctcgaccggcccgcccggcaggcccccacccggcaggccctcgcccgccgggtcgacgcgcaggtcgcgggccgccgcctcggcgcacagcacggcgagcaccgggccccgcgcggcctcggtcgtgcccagccacagcgccaccacccccgccccgcgcaggaacgaccagtgcgcgccccggtcccgcgcgcgcagctcccgcacgacggggggcagcacctcggccaccgagcgggctccaccgcctgccagcgaccagcccgaccaggacggggcgcccgccgccgggggtccgccgaggggcgctctcgcggaaccggtccgcgtcatgtccaccaccacttccgccttggcgagaacgggtcctgcgggatcaccgcgctgttccgacgccgccgacaatagcgacgcgcaatacgccgaattcaccgccaaatcaggtcaggggggttgagggggatgccttagggggcgagtgcccgcaaagcggaagaagaatcggaagcacatgcaggagcgacttccaagctcaggccgcaggaccgggtccgcgtcgtcgcggacaccccggtcctgcgcgtgcgcgcaccgaaggacgtggtgacatgcttcggaccgacctgatccgcccggttcccgaactgctcggggccaacgcggatcgcttcggcgacaggaccgcctactcggacggtcgccgttcggtcgggcacgccgggctggaacggcgcacgcgccgcctcgccggtcacctcgggcagttgcggctgcaccccggcgaccgcgcgatgatctgcctgggaaatcgcgtcgaaatgatcgagagctatttcggcgtgctccgcgcggacgccgtggcggtcccggtgaacccgcgttccaccgacgcggagctgacccacctgctcgccgacagcggggcccggctggtgatcaccgacgcggcgcgcgccgagcggttcgaccggttgcgcgccgagcggttcggcgacctgaccgtgatcgccacccaggacggcccgctgcccgacggcgtcatcgcgttcgagccgctggccgccgaggagccggagctgcccgcgcgcgacgggctcgggctcgacgacgtggcctggatgctctacacctccggcacgaccgggcgccccaagggcgtgctgtccacgcagcgcagctgcctgtggtcggtggccgcctgctacgtgccggtgccggacctgcgcgccgaggaccgcgtgctgtggccgctgccgctgttccacagcctgtcgcacatcacctgcctgctggccgccacggccgtgggcgcgaccacgcgcatcgtggacggcacgtccgcgcaggacgtgctcgcggcgctggagcaggagcggtcgacgttcctggcgggcgtgccgacgctgtaccggtacctggtcgacgccgcccgcgagcgcgggttcaccgccccggacctgcgggtgggcctggtcggcggggcggtgacgacggcggagctgctgcgcgcgttcgaggacacgttcggcgtgccgctgatcgacgcctacggcagcaccgagacgtgcggggcgatcgcggtgaactggccgaccggggcgcgcgtggcgggctcgtgcgggctgccggtgccggggctgacggtgcggctggtggacccggagacgctgctggacgtgcccgccgggcgggagggcgagttctgggtgtcggggccgagcgtgatgctgggctaccacaaccagcccgaggcgacggccgaggtgctgcgggacggctggtaccgcacgggcgacctggggcggcgcgacgaggccgggttctgcacggtcaccgggcggatcaaggagatgatcatccggggtggggagaacgtgcaccccggcgaggtcgaggccgtggtgcgggcggtgccgggggtggcggacgtcgccgtcgtgggcaagccgcacgacgtgctgggcgaggtgccggtggtgttcgtggtgccgggcgcgggcgggttcgacccggcggcggtgctggcggcgtgccgggaggagctgtcgtacttcaaggtgcccgaggaggtctacgagatcgagcgggtgccgcgcacggcgtcgggcaagaccacccggcacgtgctgctggacctgcccgcccggttgcgggcggcgtcgagcgggcagttccagtcgctgctgcggctggactgggtgccgaggacggcgctgccgggtgaggaggtcccggcgagctgggtgctggtggacggcgacccgctggggctcgcggacgggttgcgggccacgggcgcgcgggtgcgggtgggcgagccgggcgcggatgcgctgggcgacggcggatcggacgccgacgagccgggcgcgagcagcgcgggcgaaccgggctcgggtggctcgggtgagccgggctcgggtggctcgggcgaaccgggctcgggtgaaccgggcgcgagcagcgcgggtgagccgggtgcgggtgagccgggcgcggccgaacccccgcaggtcgtgctggtcgccgcggtccccggtgagcgtggtgaggtcgcgcgggacgtggaggcgctcgcggacgggctcgcgcggcggctcgtcgggtggctggccgacgagcggttcgcgggggcgcggttcgtcgtggccacctcgggcgcggtgtcgacctcccccggcgaggacctgcgggagctgcgggcggccccgctgtggggtgtggtgcggtcggtgcaggccgcgttccccggtcgggtggtggcggccgacctggacgcgtccggcgacgggcgggcggcggcgctggctcgcgtcgtcgcgggcgggcacgaccaggtggccgtgcgcggcgacgtgccgctggcgccccggctggccagggtgtccgtgccgtccgacccggcccccgccccggcgctggacccggacgggctggtcgtggtcaccggtggcgactcggcgcgcggcgcggccctcgcgcggcacctggtggccgcgcacggcgcgcggcgcctgctgctggtctcccccgacgggctgcccgaccaggccgccgccgacctggaggccgggttcgcggcggcgggcgcgcgggcggagtcggtggtgtgcgacccggccgacccggtcgcgctgcgcgccctgctcgacgcgcaggaccgcccggtcacggccgtggtgcacgtgcagggcgggcgggcgctgctggactcggcgcgcgccctcgtcgccctgcacgagctgacccgccaggcgcgaccggcgctgttcgtcgtggtcacctcggtggccgggctgctgggctcggcgggcgacccggcgcgcgcggcggccgaccagttcgccgaggcgctggtgcgcaggcgcgccgaccggggcctgccggggctggccgtggcctggggtccgctgccgggcgagcccgcgcaggcgggcgcgggcgcgctgccgatggccgaggcgctgaccctggtcgacgccgcgctcgccgccgaccagggcccgctggtggtgctcgggctcgacgcggtcgggtcgcggcgcgcggtgggcgcggtgccgccggtgctgcacgacctggtcgacggcggtcgcgccgcgcgggtcgcgccgggcgcggtggccgagttcacgcgcaggctcgcggaggcgggtgggcagcgggcccgacgcgtcgcgctggacctggtgcgcgagcacgtcgcggcggcgctcggcctgcccgaggacaccccggtgcgcgccgaccaggcgttccgcgacttcggcgtcacctcgctgaccgccgtggcgctgcgcgaccggatcaacgccgcgaccggcgcgtccctgcccgcgacggcggtgttcgaccacccgaccccggccgcgctcgccgaccacctggtgcgcgaggtcaccggcgaccggccgcacgtcgagcgggcgcgggacgagcgggcgcgcgggacctcgcgcgcggacgagccggtggcgatcgtcgccatggggtgcaggctgcccggcggcgtggcctcgccggaggacctgtggcggctggtggacgagggcgtcgacgcgatcggcccgttcccgaccgaccggggctgggacctggccaccctgctcgacggctcggactcgccggggaggtcctccgtggaccgcggtggtttcctgccgggcgcgggcgacttcgacgccgggttcttcggcatctccccgcgcgaggccctggccatggacccgcagcagcggttgctgctggaggtggtgtgggagaccgtggaacgcgccgggatcgacccgcgctcgctgcacggcgaagacgtcggcgtgttcagcggcctgatgtaccacgactacgggaccgaacccggttccgcgccggagggcctggaggggttcgtcagcaccggcagcgcgggcagcgtggtctccggccgcgtcgcctacgcgctcggcctgaccggcccggcgctgaccgtggacacggcgtgctcgtcgtcgctggtggcgatccacctggcggcgcaggcgctgcgctcgggcgagtgctcgatggcgctcgcgggcggggtcgcggtgatggggcagccgacgtcgttcgtggagttctcccggcagcgcgggctcgccgccgacgggcgctgcaagtcgttctccgacgacgccgacggcacgaactgggccgagggcgtgggcgtgctgctgctggagcggctctcggacgcgcgccgcgacgggcacccggtgctggcggtgctgcgcggcagcgcggtgaaccaggacggggccagcaacgggctgaccgcgcccagcggcccggcgcagcagcgggtcatcaggcaggcgctggcgaacgccgggctgcgaccgtccgaagtggacgccgtggaggcgcacggcaccggcaccaccctgggcgacccgatcgaggcgcaggcgctgctcgccacctacgggcaggaccgcgagcagccgctgtggctgggctcgctcaagtccaacctcgggcacgcgcaggcggcggcgggcgtcgcgggcgtgatcaagatggtgatggcgctgcggcacggcgtcctgccccgcaccctgcacgtcggcacgccctcgtccaaggtcgactggtcggcgggcgcggtcgagctgctgaccgaggccaggccgtggcgcgcgaacgggcggccacgccgggcgggcgtgtcctcgttcggggtcagcggcaccaacgcgcacgtcgtggtggaggagcaccgggaaccggccgccgcgccggtcgacccggtctcccccggcctggcggtcagcggcggcgtcgcgccgctggtgctgtccgggcgcacccgctccgcgctcgccgcgcaggccgcggccctgctggggcacctggccgacgggaccgacccggcggcgctgggccgcgcgctcgccaccacccgcaccgcgttcgagcaccgggccgcggtcctcgcgccggacgtcgacgccgcgcgcgccggggtgcgcgcgctcgccgaggaccggcccgcgccgaacctggtcaccgggcaggccgacgtggacggcccggtcgtgttcgtcttccccggccagggcgcgcagtggaccggcatgggccgggagctgctggagacctcgccggtgttcgccgcgcggctgcgcgagtgctcggaggcgctggagcggtggaccggctggtccctgctcgacctgctcgccgacggggcggagctggaccgggtcgacgtgctccagcccgcctcgtgggcggtgatggtggcgctggccgcgctgtgggagtcgtgcggggtgcgcccggacgccgtggtcgggcactcgcagggcgaggtggccgccgcgtgcgccgccgggtggctgtcgctggacgacgcggccagggtggtggcgctgcgcagccgcgcgatcgccgagcacctggccgggcgcggcggcatgatgtccgtcgccgccggggcggagcgggtggccgggctgatcgccgaccggcagggccgggtgtcggtggccgccgtgaacgggccgtccgcgaccgtggtggccggggccgccgacgcgctgcccgagctggccgcgcgctgcgagcgggagggcgtgcgggcccggatcatcccggtggactacgccagccacaccgagcacgtggacgcgctcgacggggtgctgcaggaggtgctggcgggcgtcaccgcgcaggccgggcacgtgccgtggctgtccaccgtggacggcgagtgggtcgacggctcggggctggacgcggactactggttccggaacctgcgcgggaccgtgcggttcgccgacgcggtggcggcgctggcgggctccgggcaccgggtgttcgtggaggtgtccagccacccggtgctcaccgccgcgaccggcgaggtgctggaggccgccggggtgcgcgacgcgctggtggtcggctcgctgcggcgcgacgacggtggccccgagcggttcctcaccgggctcgccgagctgcacgcgcgcggcgtcccggtggggctggaggcggtgttcgcgggcgcggacgggcgggtggagctgccgacgtacgcgttccagcacgagcggtactggctggcgcgcggcccggtggccggggacgtgtccgggtcggggctggtggacgcggcgcacccgctgctcggggcggtcgtgccgctgccgggcacgggcggggtgctgctgtccgggcggctctcgcaccggcggcagccgtggctggccgagcacgcggtggccgggacggtgctgctgccgggcgcggcgatcgtggagctggccgtgcgcgcgggcgacgagaccgggtgcggggtgctgcgggagctggtgatcgggcagccgctggtggtgccgccggacgccgaggtggacctgcaggtgctcgtcggcggcccggacgacgggggcgtgcgggacctgcggctgtactcgcggaccggggcggcggcggagtgggtcgagcacgcggcaggcgcgctcgcccccggcggcgcggtcggcggggcgcgaccggccggggcgcggacggccggggcgcgactggacggggcgcgactggacggacagtggccacccgcgggcgcggaacccgttgcgctggaaggcttctacgagaacctggcggagctgggctacgagtacgggccgctgttccgggggctcgcggcggcgtggacgcgcgacggcgaggtgttcgccgaggccgtgctgcccgaggaggcgttgtccgggcaggcgttgtccgggcaggcggggtccgggcaggcggggtccgggaacgggtccgggaacgggttcggcatccacccggccctgctggacggggcgctgcacgcgggcaacctgtgcgtgccgcccgcgccgggccggacgctgctgccgttcgcgtggaacgaggtgcggctgcacgccaccggggcgacggcggtgcgggtgcgcgtgcgggcgaccggcgaggactccctggagctggagctgttcgacgccgacggcgcgcccgtggcgagcgtcggcgggctgaccctgcgaccggcggtcacgggcgcgcgcccggccgagtcgctgcacgaggtggagtggaccgaggtcgcggcgggcggttcgtggccggaggtcgccgacacccgcgactgggaggccgccgccgacctgccgacccggtcgcgcgagctggccgcccgcgcgctggaactggtgcaggaccggctggcgggcgtggacggcgcaccgctgctggtgatcaccacgggcgcggtggcggtggccgacgacgccgaggtcaccgacccggccgccgccgccgtctgggggctgctgcgctcggcgcagtccgagcaccccggccggttcgcgctggtcgacgtcgacggcggcgcggcggccgaggtcgccgcgctcgtgcccggcgacgagccgcagaccgcgctgcgcggcgggctcgtgcgggctccgcgcctgcgccgcctgccccccggtctcgtgccgcccgccggggcgcactggcacctggacgcagtcaccaccggcacgctcgacgggctcgcgctcgtggcctcggaaccggtcccgctgcgggccggggaggtgcggatcgaggtcagggcggccgggcagaacttccgggacgtgctggtggcgctggacggcgtcgcgggccaggagggcatcggcggcgagggctccgggatcgtgaccgaggtcggccccgaggtgaccggattcgccgcgggcgaccgggtgatggggctgttcccgcgctcgttcgggccgctggccgtggccgacgcccgcacggtggtgcgggtgccgcgcggctggtcgttcaccgacgcggcggccgtgccggtcgcgttcctgaccgcgctgcacggactccaggacgtcgccgggctgcgggccggggagacggtgctggtgcacgcggcggcgggcggcgtcgggcaggccgccgtgcagctcgcccaccacttcggcgcgcgcgtgctggccaccgcgcacccggccaagcacagcgtgctgaccgcgctgggcgtgcccgccgagcggctcgcctccagccgcgacctcggctacgcgcggcggttcggcgacgtcgacgtggtgctgaactccctggtcggcgagcacgtcgacgcctcgctgcggctgctgcgcgcgggcggccggttcgtggagatcggcaagaacgacgtccgggacgccgactcggtcggggacgtccgctaccgggtgttcgacctgggcgcggacgccgggccggaccggatcggcgagctgctggagcagctggtgggcctgttcgagtcgggcgcgctgcggccactgccggtgcgcacgtgggacgtcacccgcgcggcctcggcgttccgcgagatgagccggggcgggcacaccggcaagatcgtcctgacgatcccgcgccgcctcgaccccgagggcacggtgctgatcaccggcggcgccggcacgctcggggccaccgccgcccgccacctggtcaccgcgcacggcgcgcggaacctgctgctggtcggcaggcggggccccgacgcgcccggcgcgagcgagctggcggaggagctgcgcgggctgggcgcggacgtgcgggtggcggcgtgcgacgtcgccgaccgggccgcgctcgacgccctgctcgcctcggtcccggccgggcgcccgctgacggcggtcgtgcacgcggcgggcgcgctcgacgacggcacggtcaccgcgctcaccccggagcggttcgacgcggtgttccgccccaaggtggacgcgatcgcgcacctggacgaggcgacccgcgacgccgacctggccgcgttcgtcgtctactcctcggcggcgggcgtgctcggcaacgcggggcagggcaactacgcggcggcgaacgccgtgctggacgcggtggcccgcacccggcacgcccgcgccctcccggcgacctcgctggcctgggggttgtggagcgacacgagcgcgctgaccgcgacgatggacgggcgcgcggtggaccgcacgcggcgcgcgggcgtgctgggcatgggcaacgacgaggcgctggcggcgctggacgcgggcctggcgtccgggctgcccgcgctggtggccgcccggatcgacccggccgcgctgcgcgaccccgcgtcggggtcgccgctgctgcgcgggctggtgcgcgccacccgccgcacggccgccacccgcgaccgggacgccgtgggcgggctggccggacggttggccgggttgtcggccgcggagcaggacgagctgctgctgggcctggtgcgcagcgaggccgccgccgtgctcgggcacgcgagcgccgagcgggtcgagccgcaggtggcgttccgggacatggggttcgactcgctcaccgccgtggagctgcgcaaccggctcgcggcggcgaccgggctgcggctgcccgcgacggcgacgttcgaccacccgacgccggtgcggttcgccgcgctgctgcggggcgagctgctgggcgccgtcgtggctcccggagccgtgaccgccgccgcggctcccgtgaccgccgccgcgcccgccgacgagccgatcgcgatcgtgtcgatggcgtgccggctgcccggcggggtggtcgacccggccgggctgtgggagctgctcaccggggagcgggacgggatcgtggacttccccgacgaccggggctgggacctggagtcgctctaccacccggacgccgactcccccggcacctcctacgtgctgcgcggcgggttcctggacgacgcgggcgggttcgacgccgggttcttcggcatctccccgcgcgaggccctggcgatggacccgcagcagcgggtgttcctggagacctgctgggaggcgttcgagcgcgccgggatcgacccggtctcggtgcgcggcagcgacaccggggtgttcgccgggatcatcgaccaggactacggggtgcgcgcgggcacggcccccgaggagctggagggctacctgctcaccggcaccgccacgtcggtggcgtccgggcgggtggcctacctgttcgggctggagggcccggcggtcaccgtggacacggcgtgctcgtcgtcgctggtggccacgcactgggcggtgcaggcgctgcgccggggcgagtgctcgatggcgctggcgggcggcgcgaccgtgatggggcggccgtcggcgttcgtggagttctcccggcagcgcgggctggcgcgggacgggaactgcaaggcgttcggcgcggacgcggacggcaccgcgttcagcgagggcgcgggcgtgctgctgctggagcggctctcggacgcgcggcggcgcgggcacccggtgctcgcggtgatccgggggtcggcgctgaaccaggacggggcgtcgaacgggctgaccgcgcccagcggaccggcgcagcagcgggtgatccgggcggcgctggccgacgcgggcctgcggccgtcggacgtggacgcggtggaggcgcacggcaccggcaccgcgctcggcgacccgatcgaggcgggcgcgctgctggcgacctacggcgcggaccgggagggcgcggaaccggtgtggctggggtcgctcaagtccaacaccgggcacacgctggcggcggcgggcgtgtcgagcgtgatcaagatggtgctggcgctgaaccacggcctgctgccccggtcgctgcacgtgcgggagccgagcgcggcggtggactgggagtcgggcggcgtgcgcctgctgacgagcgcccggccgtggccggagagcggcaggccccggcgggcgggggtgtcgtcgttcgggatcagcggcacgaacgcccacctggtgctggaagccgcgcctgcggaggagggcgcgggggcgcggagtggggcggcggcgccgggaccggacacccggtcggcgcccaccccggacgccccagcgggccccgtccagacctccggcgtgatcccctggccgttgtcggcccgctccgccgacgcactgcccgcgcaggccgcgaagctggccgcccacgtgcgggcgcacgacgacctctcgccgctcgacgtcggctggtccctcgcgaccacccgcaccgcgcacccgcaccgcgccgtgctcgtcggcggcacccgcgaggcgctgctgtcggccgccgacgcgctcgcgggcggcgaggccagccaggccgtgctcaccggctccgccgtcgggtcgggttcggcgaagaccgtgttcgtgttccccggccagggcgcgcagtgggcgggcatgggccgtgagctgctggggtcctcgccggtgttcgccgcgcggctgcgcgagtgcgccgacgcgctggccccgcacaccgactgggacctcctggacgtggtgcgcggcgcggagggcgcgccggggttcgagcgggtcgacgtgctccagcccacctcgtgggcggtgatggtggcgctggccgcgctgtggcgctcgtgcggggtggagccgtccgccgtcgtcgggcactcgcagggcgaggtggccgccgccgtggtcggcgggtacctggcgctgggcgacgcggcgcggctgatcgcgcggcgcagcagggccatcgcgcaggagctgaccgggcgcggcgggatgctgtccgtgctcacctcgcccgagcgggtcgccgaactgctggagccgtgggccgggaagctgtggatcgcggcggtcaacagccccgcgtccgtctcggtgtccggtgacgccgaggcgctgggcgagttcgtgcgggtgctggccaaggcccggatcaaccggtggcggctgcccggcgtggacttcgccgggcactccgggcacgtcgacggcatcgaggcgcggctgcgcgaggagctggccgacgtcaccgccgcggcgggcgaagtgccctggctgtccaccgtggacgggcggtgggtggagcgcaccaggctggacgccgactactggtaccgcaacctgcgcgacgtggtccgcttcgacgaggccgtccgcgcgctggtggacgccgggcaccgggcgttcgtggaggtctccacgcacccggtgctgaccaccgcgatcggcgaggtcgccgacgagcggcaggacgtgcgggtcgccgtggcgggcacgctgcgccgcgacgacggcggcgcggaccgggtcgtgggcgcgctcggcgaggtggccgcctcgggcgtggcggtggactgggcggcggtgttcggcgggaccggggccgcggtggtggagctgccgacgtacgcgttccggcacgagcggttctggctcaccccgtccggcggcgacgtgcgcgcggtggggctgcggcaggccgggcacccgctgctgggcgcggtggtcagcgtcccggacaccggcggcgtgctgctgaccgggcggctgtcgctgtccgcgcagccgtggctggccgaccacgcgctgtccggcgtgccgctgctgccggggacggcgctggtggagctggcggtgcgcgcgggtgacgagaccggcacgccggtggtggcggagctggtgctgggcaggccgctcgtgctgccgcgcaccgggtcggcgcaggtgcaggtgctggtgggcgaggaggcgcgggacgggcggcggccggtcgcggtgtactcgcgggcgggcgacgaccggccgtggaccgagcacgcctcgggctcgctcgcgccggacgaggacgccgcgccgggagcggagggcgacgagtggccgcccgccggggccgagccggtggacctcggcggcttctacgacggcctcgccgaacggggctacgactacggcccggccttccggggcctggtgcgcggctgggtcaggggcgacgaggcgttcgccgaggtcgggctgcccgacgaccagcacggcgcggcggcccggttcgggctgcacccggcgctgctggacgcggccctgcacgcggcctcgctgtgcgcgggccacggccggggcacggcgctgccgttcacctggaccggcgtgcggctgcacgcggccggggcgacggcgctgcgcgtgcggctggaggcggacgggccggagcggttgtcgctgcgggcgagcgatccggcgggcacgcccgtggtgaccgtcgggtcgctgctgctgcgcgccgccgacgcggaccggctgcgggcgacagcggcggcgacggcggcagcggcggcggacgacgggctgcacgcgctggagtggaccccgcacccgctgcccgaggagacgaccggttcccccgccgtcctggacaccagggcgtgggagctgcccgagggcgtcgggcgggccgaggcgatcaccacgcgggtgctcgccgagctccaggccgagctcgacgggacggcgaccctggtcgtggtgacgcggggcgcggtggccgtgcatgacgacgccgaggtcaccgacccggccgccgccgcggtgtgggggctggtgcgcgccgcgcaggccgaggaacccggacgcgtcgccgtggtcgacgtcgacgacgcctccgaggccgcgctggacgccgccgcgcacgccgcgggcgcagaaccgcagctcgccctgcgcggcggggcggcgttcgcgccgaggctggtcgaggcgtccggggcgctggccgtgccggacgggccgtggcggctcgacagcaccggccggggcaccctggagaacctggcgctcgtgcccaaccccgccgccggggcgccgctcgcgcccggtcaggtgcggatcgtggtgcgggcgggcggcctgaacttccgggacgtgctgatcgcgctcgacgcctacgagtcggagatcggcaccgagggcgcgggcgtggtcgtggaggtcgcgccggacgtcacccgcgtggccgtgggcgaccgcgtgatgggcatgatccccggctcgttcgggccgctggccgtggccgacgcccgcacggtggtgcggatgccgcgcggctggtcgttcaccgacgcggcgggcgtgccggtcgcgttcctgaccgccctgtacgggctgcgcgacctcggcggcctggcggagggcgagaccgtgctggtgcacgcggcggcgggcggcgtcggcatggccgccgtgcagctcgcccggcacttcggcgcgcgcgtgctgggcaccgcgcacccggccaagcacgccgcgctggacctgcccgccgaccacctggcctccagccgggacctcgcctacgcgcagcggttcggcgacgtcgacgtggtgctgaactccctggtcggcgagcacgtcgacgcctcgctgcggctgctgcgcgcgggcggccggttcgtggagatgggccgggcggacctgcgcgacgccgacgaggtggcgcgcgagcaccccggccgcgcctacctcccgttcgacctcggcggcgacgccgggccggaccggatcgccgagctgctggtggagctggtggccctgttcgagtcgggcgcgctccgcccgctgccgacccggcgcaccgacctggtgcgcgcccccgaggcgttccgggccatgagccagggccgccacgtcggcaagctcgtgctcaccccgccccgcgcgctcgaccgcgacggcacggtcctgatcaccggcggcacgggaaccctcggcgcggctctggcccgccacctggtggacgcgcacggcgtccggaacctgctgctggtcagccgcagcggccccaacgcgccgggtgcggccgacctggtcgcggagctggccgagcggggcgcgagggtccgggtggccgcgtgcgacgtggccgagaaggacgcgctcaccgcgctgctcgcctcgatccccaccgggcgcccgctcaccggcgtcgtgcacgcggcgggcgcgctggacgacggggtgctcaccgccctggacgccgaccgggtcgcggcggtgctgcgccccaaggccgacgccgccctgctgctgcacgaggccaccgaggacgccgacctcgcgctgttcgccctgtgctcgtcggtggcgggcgtgctgggcaacgcgggccaggcgaactacgccgccgccaacacctacctggacgcgctggcccagcaccggtcggccgccggtctggccgcgctgtcgctggcctggggccggtgggcgcagaccagcgccctcaccgcagacctgcccgcgcccggcggtcgccgcgacctggtgcgccccatggacaccgcgtccgcgctgcgcctgctcgacgccgcgctccgcaccggacgctcgacggtcgtcgccgccgagctggacgtcacggcggccaccgccgcgaacccggtgctgcgcggcctggtccggcccgcccggcgcgcgctggccacgtccgcgcgggacgagcgcggcgtggcggcggcgctggccgggctgggcgaggccgaccggcgccggttcgtgctggacctggtgcgctcgcacgccgccgtcgtgctgggcctggcgggcaaggaggccgtggacgccgagcgcgcgttcaccgagaccggcttcgactcgctcaccgccgtggagctgcgcaaccggctcgccgccgcgaccgggcttcggctgccctccacgctggtgttcgaccacgccaccccgaccgcgctggccgcgcacctgcgcgccgagctgaccggcgacgacctgccgcaggcgcgggccgtcgccgccacggcgggggcgcgggacgacgacccggtggtgatcgtgtcggcgagctgccgcctccccggcggcgcggactcgccggaggcgctgtgggagctgctggagcggggcagggacgccatcaccccgttcccgcgcgaccggggctgggacctggaggcgctctacgacgccgacccggaccggccgggcaagagctacgtgcgcgacggcgggttcctcgccgacgcggccgggttcgacgccgagttcttcggcatctccccgcgcgaggcgctggccaccgacccgcagcagcggctgctcgccgagacctcctgggagctgttcgaacgcgcgggcatcgccccgacctcggtgcgcggcagcgacgtcggcgtgttcgcgggcgtgatcaaccaggagtacggcgtgcacagcggcacgacccccgccgagctggaggggtacgtgatgaccggctcgaccaccagcatcgcctccggccgggtggcgtacctgctcgggctgaccgggcccgccgtcaccgtggacaccgcgtgctcctcgtcgctggtggcgatccacctggcggcgcaggcgctgcgctcgggcgagtgctcgatggcgatcgcgggcggcgcgacggtgatcgcgaggccgggcgggttcgtctcgttctcccggcagcgcggcgcggcccccgacgggcgctgcaaggcgttcggcgacggcgcggacggcatggcgttcgccgagggcgtcggcctggtgctgctggagcggctctcggacgcgcgccgcaacgggcacccggtgctggcggtcgtgcgcggcacggccctgaaccaggacggcgcgtccaacggcctgaccgcgccgaacgggcccgcgcagcagcgggtgatccggcaggcgctggccaacgccgggctgtcccccgacgaggtggacgcggtcgacgcgcacggcaccggcaccgcactcggcgacccgatcgaggcgcaggcgctgctcgccacctacgggcgggaccgggacccgcggcggccgctgtggctggggtcggtgaagtcgaacatcgggcacacccaggcggcggcgggcatcgcgagcgtgctcaagatggtgctggcgatgcagcggggcgtgctgcccgcgaccctgcacgccgacaccccgacgacgaaggtcgactggtcctcgggcgcggtggcgctgctgtcgcgggcgcggccgtggccggagaccgggaggccgcgccgggcgggcgtgtcctcgttcgggatctccggcaccaacgcgcacgtgctgctggagcaggccccgcaggacgcgcccgccacgccggtcgccccgcggggcgccgggctggtcggggcggtggcctggccggtgtccgggcgcacgcccgccgcgctgcgcgcccaggccgccaggctcgggacgcacctggcgggcgcgcaggccggacccgccgacgtgggctggtcgttggcgggcacgcggacggcgttcgcgcagcgggcggtcgtggtggccgggacggcggagcaggcccgtgacgggctggcggcgctggccgaaggccgctcgtccgcgctcgtgacgaccggtgaggccggggtcgacgggcgcgtggtgttcgtgttccccggccaaggggcgcagtggatcggcatgggcgcggagctgatcgacgcgtcgccggtattcgccgagcggttgcgcgagtgcgcggaggcgctggaaccgttcgtggacttcgacctgatcgaggtgctgcgcggacgcgggtcgctggagcgggtcgacgtggtgcagcccgcgtcgtgggcggtgatggtgtcgctggcagcgctctggcggtcgctgggcgtggaaccggacgccgttgtcgggcactcgcagggcgagatcgcggcggcggcggtcagcggggcgctcagcctgcccgacgccgcagccgtggtcgcgttgcgcagcaaggcgatcgcccaggacctggccgggctcggcggcatgatgtccgtcgccctgcccgccgacgacgtcgacctgagcgggtatcccggacgcctgtgggtcgccgcgcacaacggccccacctcgaccgtggtggccggtgacgtggacgcgctgcgcgagctccacgcccactacgagggcgccgaggtccgggcccggatcatccccgtcgactacgccagccacaccgggcacgtcgacaccatccgcgagcggctcgccgaggcactggcgcacgtgcggccgagggcgggcacgatcccgtggctgtcgaccgcgaccggcgagtggaccaccggtgaggacgccgacgccgactactggttccgcaacctgcgcggcgcggtgggcttccacaccgccatcaccaccctcgccgagcagggccaccgggtgttcgtggaagtctccagccaccccgtgctcaccaccgccatcgaggccacgctcgaaggaaccggacccaccgccgtcaccggaaccctccgccgcgacgacggcggccccgaccgcctcctcaccagcctcgccaccctgcacgtgcgcggcgtccacgtcgactgggacgcggtctacgcgggcagcggcgcgcaccgcacgacgctccccacctacgcgttccagcacgagcgctactggctcaccgagccggacgcgccgcaggccgtcgcggacgccccgttctgggacgccgtggacagcggcgacgtggccgcgctcgccgggtccctgggcgtcgagcccgccgccctggagccggtgctgccggggctgacgagctggcgggcccgcaaccgggacggcgcggccgtggacgactggtcctaccggatcggctgggagcgggtggacgtgcccgccgccccgctgtccgggacgtggctggtcgtggtgcccgaggcactcgccgacgacacctcggtcgccgaggtcgcggcggcgctggccgcgcgcggcgcgacgccgaggatcgtggcggcgggcccggacctgggcccggacctgggtgacgagccggacggggtgctgtcgctgctggcgtgggacgaccgcccggccgggggcggcacgctctcgcgcggcgtcgtggacgcggtcgggctggtgcgggaggcggtgcggcgcggctggtcggccccgctgtggtgcgccacgctcggcgcggtcgccgtcgccgaccccggcgaggtgacggccgagttcgggccgcagctgtggggcacgggcgtcgtgctgggcctggacctgccggacacctggggtggcctggtcgacctgcccgcgcggccggacggggtcgcgctggacctgctgtgcgcggtggtcgcgggcgcgggcgacgaggaccagctggcggtgcgcccggccggggtgttcgcgcggcgcatgacccgacgcccggtcgcgtcggcgcccgcgtggcgaccgcgcgggacggtgctggtcaccggcggcaccggcggcctcggcggctacgtcgcccggtgggcggcggagcggggcgcgcgggacgtggtgctgctctcgcgcggcggcccggacgcgccgggcgcggacgccctggtcgccgacatcacggcggcgggcgcccgctgcgcggtgctggcctgcgacgtcaccgaccgggacgcgctggccgaggtggtcgcgaacctgccggacgggccgctgtcggtggtgcacgccgcgggcgtggcgcgaccgggacggccgctggtggagaccacgccggaggagttcgcggccatcggccggggcaaggtcgcgggcgcccgcctgctggacgagctgctgggcgaccgggagctggacgcgttcgtgctgttctcctccggcgcggcggcctggggcagcggcgggcaggccgggtacgcggcgggcaacgccttcctggacgggctcgcgcagcgcaggcgcgcccgagggctcgcggccacctcggtggcctggggcgcgtggggcggcgtcggcacggtcgacgaggtgctgggcgagcagtggcggcgcgccgggctgctcaccatggacccgcgcctggccaccctcgccctcgcgcacgccgtgggctcgggcgaggcgcacctgctcgtcgcggacgtcgactgggcccgcttcgcccccgcctacgcgctggccaggccgcgcccgctgctggcggcgctgcccgaggtcgccgacgcgctggcggtcgtggacgcgcccgccgacgccggggggatcggggcgcggctggccgggctgccgcccgccgagcaggagcggctgctcaccgagctggtgcaggcggaggcggcggccgtgctgggcctgggcggcatcaccggcgaccgggcgttccgggaggtcgggttcgactcgctcacggccgtggagctgcgcaaccggctcggcgcggccacgggtctcaccctgcccgcgacgctggtgttcgaccacccgcgcccgagcgccctggccgcgcacctgcggtccgcgctgggcccggccgccgcgccggtggactcggtggcgggcgtgctggccgagctggaccggctggaggcggccatcccggcgctgccgtcggccgagatcggccggtcccggctggagctgcggctgcggcggttgagcgcccgcgtcggcgagctggtcgccgcgaacggcgagcgggcgaacggcgggcgcgcgaacggcgggcgcgcggcggccgacgagctggacgacgcgggggccgaggacgtgctcgcgttcatcgaccgggagttcggggacgcgtgagcggccacacgagccccgaccccggcccccaccgcggcccccacaacgacgaccctggcgaggaacagatggcgaacgacgagaggctcctcagctacctcaagcgggtcaccgccgacctgcaccgcacgcgggagcggctgcgcgaggcggagtccggggcggacgagccgatcgcgatcgtcggcatggcctgccgcttccccggcggcgtgcgcaccccggacgagctgtgggagctggtggcgtccggccgcgacggcatcggcccgttcccggacgaccggggctgggacctgggcgcgctgttcgacccggaccccgacgccaccggccgctcctacgtcaccgagggcgggttcctggacgacgcggccctgttcgacgcgggcttcttcgggatctccccgcgcgaggcgctggccaccgacccgcagcagcgggtgctgctggagaccgcgtgggagaccttcgagcaggcgggcatcgacccgacctcgctgtccgggcaggacgtgggcgtgttcaccggggtcgccaacggggactacgcgctgaccgtggaccgggtgccggagggcttcgagggctacctgggcatcggcggggcgggcagcatcgcctccgggcgcatctcgtactccctgggtctggagggtccggccgtcacgctggacaccggctgctcgtcgtcgctggtcgcgatgcactgggccgggcacgcgctgcgggcgcgggagtgctcgctggcgctcgcgggcggcgtgatggtgatggcgacgccgggtgggttcgtcgggttctcccggcagcgcgggctggcccgcgacgggcggtgcaagtcgttcggcgacggcgcggacggcacgtcgtggtcggagggcgtgggtctgctgctgctggagcggctgtcggacgcgcgggccaacgggcacgaggtgcttgcggtggtgcgcgggtcggcgatcaaccaggacggggcgtccaacgggctcaccgcgcccaacgggccgtcgcagcagcgggtgatccgcgcggcgctcgacgcggcggggctcgggcacgcggacgtcgacgcggtggaggcgcacggcaccgccacggtgctcggcgacccgatcgaggcgcaggcgctgctgaacacctacgggcggcaccgggacggggcgcagccgctctacctggggtcggtcaagtccaacctcgggcacacccaggcggcggcgggcgtggccggggtgatcaaggcggtgcaggcgatgcgccacggcgtgctgccgcccaccctcaacgtcggcacgcccaccaccaaggtcgactggtcctcgggcgcggtggaggtgctggcggaggcccggccgtggccggagaccgggcgtccgcgccgggtgggcgtgtcgtcgttcggcgtgagcggcaccaacgcgcacgtgatcctggagcaggcacccgagcacgagccagcgccggaggagccgggtgggcgcgggccggtggcggcgggcggcgcgacgccgtggacgctgtccgggcgcacgcccgccgcgctcgccgaccaggcgcggcggctggccgggcacgtgacggccgacctgcgggcggaggacgtcgggttctcgctggccaccaccagggcgcacctggagcaccgggcggtggtggtcggctcggacgggctggcggcgctggccgaaggccgctcgtccgcgctcgtgacgaccggtgaggccggggtcgacgggcgcgtggtgttcgtgttccccggccaaggggcgcagtggatcggcatgggcgcggagctgatcgacgcgtcgccggtattcgccgagcggttgcgcgagtgcgcggaggcgctggaaccgttcgtggacttcgacctgatcgaggtgctgcgcggacgcgggtcgctggagcgggtcgacgtggtgcagcccgcgtcgtgggcggtgatggtgtcgctggcagcgctctggcggtcgctgggcgtggaaccggacgccgttgtcgggcactcgcagggcgagatcgcggcggcggcggtcagcggggcgctcagcctgcccgacgccgcagccgtggtcgcgttgcgcagcaaggcgatcgcccaggacctggccgggctcggcggcatgatgtccgtcgccctgcccgccgacgacgtcgacctgagcgggtatcccggacgcctgtgggtcgccgcgcacaacggccccacctcgaccgtggtggccggtgacgtggacgcgctgcgcgagctccacgcccactacgagggcgccgaggtccgggcccggatcatccccgtcgactacgccagccacaccgggcacgtcgacaccatccgcgagcggctcgccgaggcactggcgcacgtgcggccgagggcgggcacgatcccgtggctgtcgaccgcgaccggcgagtggaccaccggtgaggacgccgacgccgactactggttccgcaacctgcgcggcgcggtgggcttccacaccgccatcaccaccctcgccgagcagggccaccgggtgttcgtggaagtctccagccaccccgtgctcaccaccgccatcgaggccacgctcgaaggaaccggacccaccgccgtcaccggaaccctccgccgcgacgacggcggccccgaccgcctcctcaccagcctcgccaccctgcacgtgcgcggcgtccacgtcgactggaaggccgtgttcgccggcacgggcgcgcgccgcgtcccgctgccgacctacgcgttccagcacgagcgctactggctggaccggggcgcggcggccggtgacgtcacgggcgcgggcctggccgacgcggcgcacccgctgctggccgccgtcgcccagctgcccggcaccggcggggtgctgctgagcgggcggttgtcgcgggcgacgcacccgtggctggccgagcacgtggtgaacgggaccgcgctggtgcccggcacggccctggtggagctggcgctgcgcgcgggcgacgaggtggacgcgcccgtgctgcgcgagctggtgatcacccggccgatgccggtgccggagcggggtttcctgcacgtgcaggtggacgtcgcgggtgcggcggacgacgggtcgcgggcggtgcggatctggtcgcgcgccgaggacgcggcgagcgagacggcccgctggaccgagcacgccaccggctccctcgcccccgacgacgcggccccgcccgcccgcgcgagcggcgcgtggccgcccgagggcgcggcggccgtggacgtggacgacttctacgaccgcctcgcgggcgcgggctacgagtacgggccgctgttccagggcctcaccgccgcgtgggccggggacgggcaggcgtgggccgaggtggtgctgcccggtgaggcgggcgggttcggcgcgcacccggcgctgctggacgcggcgctgcacgtgggcacgttctgcctgcccggcgggccggggtcgcgcacgctgctgccgttcgcgtggacgggcgtgcggctgcacgccaccggcgcgacggcggtgcgggtgcacgcccgcgccaccggcgacgacggcctcgtcgtggagctgcgcgacgcggacggggaaccggtcgtgacggtcgacgcgctcgtgctgcgcgacgccgaccccgccgacgcgcaggccccggacgtcacggcggacgcgttgtggcgggtgcggtgggtcgagcagccgcccgcgcccgcggcgcccggctgggtgctgctgggcgggccgtccgggcacgccgggttcgccgccctgccggtgttcgccgaccctgcggccgtggcggcgctgcccgagggcgaccggcccgcggtggtcgtcgtggacaccaccgcgtgtcgggagccggggcgggacgtgccgggggcggcgcgggcgttcgtggtgcgggcgctggagctgctggtggcgtggctgcgcgaggacgcgctggccgggacccgactggtgctagtcaccagcggcgcggtgccggtgcgcgcggacgccgaggtcaccgaccccgctgccgcggcggtgtggggtctgccgcgctcggcgcagtcggagcacccggaccgggtgtggctgctggacgtcgacgagccgggcgcggcgccgggcgcgctggcctcgccggagccgcagctggccgtccgggcgggcgcggggttcgcgccccggctcgccagggccgaggccgcgcccggcgcgctgccggtcgacgggccggtgctggtcaccggcggcaccggcacgctcggcgcgctcgtggcccggcacctggtcaccgcgcacggcgcgcggaacctgcacctggtgagcaggcgcggcccggacgcgtccggcgctcgagaactcctggacgagctgcgcgggctgggtgccgaggtcgagctgtcggcgtgcgacgtggccgaccgggtggcgctcgccgccctgctggggcgcgtgcgccccgccgccgtggtgcacgcggcgggcgcggtggacgacggcctgctcaccgacctgaccgccgaccggttcgacgccgtgctgcggcccaaggtcgacgcggtcgcccacctggacgacctactcggggacgtgccgctggtggtgttctcctccgcgaccggcaccctcggcacccccggccaggcgaactacgccgcggccaacgcggtcgccgacgcgctcgtgcagcgccgccgcgcgcggggcctgccgggcgtgtcgctggcgtggggcctgtggtcggacaccagcgagctgaccgcgaccatggacgccgccgacgtggcccgcacccgccggggcggggtgctcggcctggacgcggcgcgcggcctggcgctgctcgacgccgcgctcggcgcggacgacgcgctgctcgtgccgatccacctggacaccgccgcgctgcgccggggggccgacccggctccgccgctgctgcgcggcctggtccgccgcgcccggcgcgcggcgggcgcggcccggcaggccgcgctgccgctggtggcgcgactggccggggtggacgcggcggagcggcggcgggcgctggtggagctggtgcgcgccgaggccgccgccgtcctcgggcacggcggcccggacggcatcgggcaggaccagccgttccgggaggtcgggttcgactcgctcacggccgtggagctgcgcaaccggctcggcgcggccacgggtctcgcgctgcccgcgacggtggtgttcgaccacccgacgtccgcgcgggtcgccgagcacctgcgggagctgctgttcggcgcggagacggctcaggcccccgcgcggcgggaggtggtggccgacgacgacccgatcgccgtggtgggcatggcctgccggttccccggcggggtcgccgacgcggacgggctgtggcggctggtcgccgaggagcgcgacggcatcggcccgttcccggacgaccggggctgggacctggcggcgctgttcgacccggaccccgaccacgcgggcacctcgtacgtgcgggaaggcgggttcctcgacggggcgaccgggttcgacgcgccgttcttcggcatctccccgcgcgaggcgctggccatggacccgcagcagcggctgctgctggaggtggcgtgggagacgttcgagcaggcgggcatcaacccgcgctcggcgcacggcaccgacaccggggtgttcgcgggcgtgatctaccacgactacggcgaggcggcgggcgagctgcccgagggcgcggagacctaccgcagcaccggcacgtcgggcagcgtggtgtccggccgcgtcgcctacgcgctgggcctgaccggcccggcgctgaccatcgacacggcctgctcgtcgtcgctggtggcgatccacctgggcgcgcgggcgctgcgggcgggcgagtgctcgatggcgctggtcggcggggtgacggtgatgtcgacgccgggcgggttcgtgagcttctcgcggcagcgcgggctggccccggacgggcggtgcaagtcgttctcggagggcgcggacggcaccgggttcagcgagggcgtcgggctggtgctgctggagcggctgtcggacgcgcgggccaacgggcacgaggtgcttgcggtggtgcgcgggtcggcggtgaaccaggacggggcgtccaacgggctcaccgcgcccaacgggccgtcgcagcagcgggtgatccgcgcggcgctcgacgcggcggggttggggcacgcggacgtggacgcggtggaggcgcacggcacgggcaccaccctcggtgacccgatcgaggcgcaggccgtgctcgccacctacgggcaggaccgcgagcagccgctgtggctgggcacgctcaagtccaacctcgggcacacccaggcggcggcgggcatcggcagcgtgatcaagatgatccaggcgatgcggcacggcgtgctgccgcgcaccctgcacgtcaccgagccgaccacggccgtggactggggcgcgggcgcggtggagctgctgacgcgggcgcgggagtggccggagaccgggcgtccgcgccgggcgggggtgtcgtcgttcggggtgagcggcacgaacgcgcacgtgatcctggagcaggcccccgaaccggtggcggtggaggcggcgccggaggcgggggtgctgccgtgggtgctgtcggcccgcacgcccgaggcgctgcgggagcaggccgaccggctcgtggcgcacctgggcggtgagtcgtcctcggcggccgtggcccggtcgctggtgctgggtcgggcggccctggaggagcgggccgtggtcgtgggcgaccgggcgcgcgccggggaggcgttgcgggcgctggccgaggggcggccctcccccgcgctcgtcaccgggcggaccggggtcgaggggcgcgtggtgttcgtgttccccggtcagggcgcgcagtgggtcggcatggggcgtgcgctgctggacgcctcgccggtgttcgccgaacgcctgcgcgagtgcgcggcggccctgcgcccgtacaccgactgggacctggtcgaggtgatcacctcgggtggcgcgctggacgacgtggacgtcgtgcagcccgcgtcgtgggcggtgatggtgtccctcgcggcgctgtggcgctcgctcggcgtcgaaccggacgcggtgatcgggcactcgcagggcgagatcgccgccgcgaccgtcgcgggctggctcagcctccaggacggcgcgaagatcgtcgcgctgcgcagccagctgatcgacgaggagctgaccgggctgggcggcatgatgtccgtcgccctgcccgccgaggacatcgacctgagcggttacgagggccggttgtgggtcgcgacggtcaacgggccgagcgcgaccgtggtcgccggggacaccggggcactggaggagctgcggcgcggctgcggcgaggcggtccgcacgcgggtgatccccgtggactacgccagccacaccgggcacgtcgacgccatccgcgaccagctcgcccggatgctcgccgacgtcaccccgcggcccggcgagatcccgtggctgtccacggtgaccggcgagtggatcacccccggcgacgacgacgccgactactggttccacaacctccgccgcaccgtccacttcgccgacgggatcaccaccctgctcgacgccgggcaccgggtgttcgtggaggtctcctcgcaccccgtgctggcggcggcggtgcaggagagcgccgaggcggccggggacgcgcgggtcgccgtgaccggcacgctgcgccgcgacgacggcggctgggaccgggtcctgaccggcctggccgagctgcacgtgcgcggcgtggacgtggactggacgcgggtgctgcccgaggcggggcgggcgccgctgccgacgtacgcgttccagcacgagcgctactggccggaacccgcgcgcccggccgccgcgccgggcggtggtgacgacgcgctgtgggcggtgatcgagggtggtgacgcggcggacctggccggggagctggccgtggacgaggacgagctggcgcgggtgctgcccgccctgacctcctggcggcggcgcagccgggcaaggagcgcgctcgacggctggcgctaccgggtcgactgggtcccggtccccacgagcgggtctgggctgcccggcgggcaagcgctgtccggcgggcaggcgctgtccggcgggccgaggtcgtccggcggggcagggctctccggcggtcaggggacgccaggcgggtccgggtcgcccggcggagcggcactgccaggcgggccagggtcgcccggcggagcggcgctgcccggccgggtggccgtggtggtgcccgcgaacgacgagcgggcgcgggcggtcgcgggcgggctggtcgcgcggggtgtggacgtgaccgtcgtggcggcggtcgacgccacccgcgacgggctggcgaaggcgctgcccgaccgccccgacgccgtggtgtcgctgctgtcctgggacgcgggggccgacgagccgggcgcgcccggttcggccacggccgcgctggtgcaggccctggccgaccggggtgccaccgggccgctgtggtgcgcgaccgggggcgcggtgagcgtcgcgggcgaggacgccgaccccgaccaggccgccgtgtgggggttgggcggggtgctggccctggacctgccggaggcgttcggcggactggtcgacctgccgcggcagcccaccgacgccgacctcgacgcgttcgccgccgcgctgaccgcccccggcggcgaggaccagctcgcggtgcgcgacggccgcctgctggcccgccgcctggtccgcgacggggccgacgcgccggagtggacgccgcgcggcgcggtgctggtcaccggcggcaccggcggcctcggcacgcacgtggcccgctggctcgcccgctccggggccgggcacgtcgtgctcgccagccgctccggccccgccgcccccggcgcggccgggctggccgccgaggtggaggcgctgggcgcgcggtgcagcgtggtggccctggacgtggccgaccgggacgcggtggccgccgtgctcgccgacgtcgagcgggacgggccgctgaccgccgtggtgcacgcggcgggcgcgggactggccccgacgccggtggtggagctgaccgccgggcggtacgcggacgtcgcggccggcaaggtcgagggcgcgcgggtgctggacgaggtgctcgccgaccgggcgctggacgcgttcgtgctgttctcctccggcgcggccgtgtggggcagcggcgggcaggccccgtacgcggcggccaacgcgttcctggacgggctggccgcccgcaggcgggcgcgcgggctcgtggccacctcggtggcgtggggcggctggggcggcggcctcggcatgatcggcgacggggacgcggagcggtgggcccggctgggcatccgcacgatggacccggaggcggcgctgcgcggcatggcgctggcggtcggctccgggcgggccgcgagcgtggtggccgacgtcgactgggcccggttcgcccccggctacgccctggcgcgggagcgcccgctgctgcgcgggctgccggaggtggtggcgctgctggccgaaccggacgagcccgccgcggcggtggacgcgcggggcgcgctggcggcccggctgaccgggctggacgcggccgggcaggacgagctgctcgcggacctggtgcgggcgcaggcggcggcggtgctggggttcgccgaccctggcgcggtcgcggcggaccgggcgttcaaggacgccgggttcgactcggtgaccgccgtggagctgcggaaccggctgggcgcggccaccgggctgcggctgccgccgaccgtggtgttcgaccacccgaaacccctggctctggcgcgcgtgctgcgcgccgagctggtcccccagcggggggacggggtgacggcggcgcaggtggcgcaccgggaggacgcgatccggcgggtgctggcgtcggtgccgctggcccggttcgaggagctgggcgtgctcggcgcgctcgtggacctcgtgcccgccgcgccaccggcgggcggcgcggcgacagcggagcgggacgacctggcggacctggcggagctggacctggacggtctggtccgcagggcgatgcgcggcaccaccgccgggaacgactgaggctttgatgcggagcggagagagcatgagcgcgggcacctcgccggagagcgtcgtccaggccctgcggaccacgctggtggacaacgagcggctgcggcgggagaacgagcggctggtcgccgaggccggtgagccggtggcgatcgtgtcgatggcgtgccggctgcccggcggcgtcaccgacccggagtcgctgtgggagctggtgcgcgagggccgggacgccatcgggccgttcccgaccgaccggggctgggacctggggtcgctgttcgacgacgacccggacgcggcgggctcctcgtacgtgcgggagggcgggttcctggcgcgggcgggcgggttcgacgcgccgttcttcggcatctccccgcgcgaggccctggccatggacccgcagcagcggctgctgctggaggtggcgtgggaggccgtggagcgggccgggctcgacccgcgctcgctggagggccgggacgtcgcggtgttcgcgggcggcaacccgcagggctacggcggcggaccgggtgacgccccggagggcctggaggggttcctgggcgtcaacgcctcgtcgtcggtgatctccgggcgggtctcctacaccctgggcctgaccggcccggccgtcaccgtggacacggcgtgctcgtcgtcgctggtggcgatccacctggcggtgcggtcgctgcgctcgcgggagtgctcgatggcgctcgcgggcggggtgaccgtgatggggcagccgaccgcgttcgtggagttctcgcggcagcgcgggctcgccccggacgggcggtgcaagtcgttcggcgacggcgcggacggcacgtcgtgggccgagggcgtcggcgtgctgctgctggagcggctctcggacgcgcggcgcgacgggcacgaggtgctggcggtgatccccggctcggcggtgaaccaggacggggcgtccaatggcctgaccgcgccgaacggcccgtcccaggagcgggtgatcgcggcggccctggccgacgccggtctcggcctggccgacgtggacctgctggaggcgcacggcaccggcaccaggctgggcgacccgatcgaggcgcgggcgctgctcaacacctacggccggggcaggccgcaggaccggccgctgtggctggggtcggtgaagtcgaacctcgggcacgcccagtcggcgtcgggggtggcgggcgtgatcaaggtggtgcaggcgatccggcacggcctgatgccgcgcacgctgcacgccgacgagccgagctcgaacgtggactgggcggcgggggcggtggagctgctggcgcgcgagcgggagtggccggagaccgggcgggcgcggcggggcgcggtgtcgtcgttcggggtgagcggcacgaacgcgcacgtgatcgtggagcaggcgcccgaggaggccgccgccggggtcgcggcggcggggcggcccgcgcccaggtcggcgggcgggcaggacgccgggatcgcggcggtgaccgggcaggccgcccccgccgctggccccgccaccgccgaacccgccgcgtcggccgtcgaggacgggaccggcgtcgcccccggcccggtcgcgaccggcggggtcgtgccgtgggcgctgtccgggcggaccgccgccgcgctggccgcccaggcggcccggttgcgcgcgcacctcgccgcgcacccggcggcccgcccggtggacgtggcctggtcgctggccacgacccgctcggtgctggagcaccgggccgtcgtgcccgccgcctcgctcgacgaggccctggcggggttggacgcgctcgcctcgggccgcgcggaccggtcggtggtcgtcggcgaggcggcgcccggccgggtggcggtgctgttcaccgggcagggcagtcagcgggccggtgcggggcgcgagctgcgggagcggttcccggtgttcgcgcgggcgttcgacgccgcgtgcgccgccgtgggcgagctgcccaccggcgacggcggcgcgatcgggctcgccgaggtggcgctggccgaccccggcacgcccgccgccgcgctgctcgaccggaccgcgttcacccagcccgccctgttcgcgctggaggtcgcgctgttccggctggtccagtcgtggggcgtgcgcccggcggcgctggccgggcactcggtcggcgagatcgccgccgcgcacgtggccggggtgctctccctcgccgacgccgccgcgctggtgcgcgccaggggcgggctgatgcaggagctgcccgagggcggcgcgatggtggcggtggaggcggccgaggacgaggtcgtgccgctgctcggggacggggtgtcgctggccgccgtcaacggcccgacctcggtggtgctctccggcgacgaggaggccgtcaccgccgtcgccgcgaggctggcgcagcggggcaggcgcaccaagaggctcgccgtctcgcacgccttccactcgcaccgcgtggacccggcgctggccgccttccgcgccgtggccgaggagctcgcctacgccgcccccacgatcccgatcgtctccaccctcaccggccgccccgtcacccccgacgagctgcgctcccccgactactgggtgcggcacgcgcgcggcgccgtccggttcctggacgccgtgcgggcgctgggggacgcgggcgcgcgcacgttcctggagctgggcccggagggcgtgctcacggcggcgggcgcggactgcctgccggacgcggtgttcgcggcgacgctgcgcgccgacgtgcccgaggcgcgggccgtgctcgccggggtcgcgggcctgcacgtgcgcggcgcgacagtcgactggggttcgctgttcacgggcgcggacgcgcggcgcgtcccgctgcccacctacgcgttccagcacgaggaccactggctggtgcgccgctccaccgccgccgacgtgggcgcggtcggcctgcgcgaggccgggcacccgctgctgggcgcggtcgtcgcgctgccggagagcggcggggtgcagctgagcggtcggttgtcggtggcggcgcagccgtggctggccgagcacgtcgtctccggcacggcgctggtgccgggcgcggcgctggtggagctggcggtgcgggcgggcgacgagaccggcacgcccgtgctggaggagctggtgatcggccgcccgatgccgctgccggacggcggcgcgctgagcgtgcaggtcgtcgtcggcccggacgagggcgggcgccggtcggtgcgcgtgtactcccgcgcggacggggcggtggactgggtcgagcacgcggcgggggcgctgaccgcgccggaggccgcgccgaccgccgacgcgggcccgtggccgccggagaacgccgaacccgtggacacgcggggcttctacgacaccctcgcggagggcggctacgcctacggcccgctgttccggggcctgacctcggcgtggcgcggcgagggcgaggcgtgggcggaggtggcgctgcccggtgacgcgaccgggttcggcatccacccggccctgctcgacgccgcgctgcacaccgcgcacttctgcctgcccaccgggaccgagcggcgggccgggctgctgccgttcgcctggaccggcgtgcggctgcacgcgggcggcgcgacgaccgcgcgggtgcacgcccgcgccaccggcgacgacggcgtgaccgtgcgcctgctcgacggtgccggtcagccggtcgcggacgtggccgccctgaccttccgcgccgcagccgacaccccgtccgccgaggtcccggacgcgctgtgggcggtggagtggaccgagcacccgctgcccgcggacgggaccacccccgcgggcgggaccaccacggccgtggtggtcgtggacacccggagcgtcgacgcccccgacgacggccccgcccgcgcccgcgcgctgaccgcccacgtcctcgccgagctgcagcggcacgccgacgacgaccggccggtcgtcgtggtcacctcaggcgcggtcgccgtgcgcgtcgacggcgaggtcaccgaccccgcgtccaccgccgtgtgggggctggtgcgggccgcgcaggtcgagcagcccgaccgggtccggctggtcgacgtcgagccgggggccgacccggtgctcacctcgcccgagccgcaggtggcgctgcgcggcgggaccgcgcacgtgcccaggctggtccgcgcccgccgcgccctcccggcgccgaccgcgacgtcgtggcggctgggctccgaccgccccggcacgctggactccctcgccctgctcccggacgactccggcacggccccgctcgcccccggcgaggtgcggatcgcggtccgcgcggcgggcctgaacttccgcgacgtgctggtcgcgctggggatgtaccccggtcgcgcggtgatcggcgcggagggcgcgggtgtggtcgtggaggtcggccccggccccgacgacaccgacgccggcgacaccggccccggcgacaccggctcgggcggcctggccgtgggcgaccgggtgatgggcctgttccccggcgcgttcggcccgctggccgtggccgaccaccgaatggtgacccggatgccggacggctggtcgttcaccaccggcgccggcgtgcccatcgcgttcctgaccgccctctacgggctgcgcgacctcggcgggctcaccgcgggcgagaccgtgctggtgcacgcggcggcgggcggggtcggcatggccgccgtgcagctcgcgcgggcgttcggcgctcgggtgctgggcaccgcgcacccggccaagcacgcggccgtgacccgcctgggcgtccccgagtcccacctgtcctccagccgcgacaccgcctacgccgacctgttcggcccggtggacgtggtgctgaactcgctcaccggcgagcacgtggacgcctcgctggggctgctgcgcgcgggcggccggttcctggagatgggcaagaccgacctgcgcgacgccgacgaggtcgcgaaggcgcaccccggcgtcgcctaccgcccgttcgacctgggcggcgaggcgcccgccgagcgcgtcgcggagctgctggccgagctggtcgcgctgttcgaggcgggccgcatccacccgctgcccaccgcggcctgggagatcacccgcgcgccggaggcgttcggctggatgagccgggccgggcacgtgggcaagatcgtgctgaccctcccccgccgccccgacccggacggcacggtgctggtcaccggcggcaccggctcgctcggcgcggtcgcggcccggcacctggtcaccgcgcacggagcccgccacctgctgctcgcctcccgacgcggcgagcaggcccccggcgcggcggagctgaccgacgggctgcgcgggctgggcgcggacgtgcgggtcgcggcgtgcgacgtcgccgaccgggacgcgctcgccgcgctgctcgccacgatccccgccgcgcacccgctcaccgccgtcgtgcacacggcgggcgtgctcgacgacggcgtgctcgccgcgcagacccccgagcgcctggacgcggtgttccgccccaaggtcgacgccgtcgcgaacctgcacgagctgaccggcgacccggccctgttcgcggtgtactcctcggcctccggcgtgctcggcggcgcgggccagaccaactacgccgccgcgaacgcctggctcgacggcctcgcccacgtccggcgcgcggcgggcctgcccgcgacctcgctggcctggggcctgtgggcgcaggacggcggcatgacgggcggcctggcgggcggaccggccgggccgggcgggcgggcccgccggggagccgtcgcgccgctgtccaccaccgagggcatggcgctgttcgacgcggccgtcgcgtcgggccgcccgctcctggccccgatcaggctcgaccccgccgcgctcaccgccgacggcgcgcagccgcccgcgctgctgcgcggcctggcccgccccacccgccgcaccgccgtcgcggccaccaccgacgacggcctcgcgggcaggctcgccgcgctcgacggccccggcaggcagcggctgctcgtggagctggtgcgggagcaggccgccgccgtgctgggcttcgcgaccccggacgccgtgtcgccgggccgggcgttccgggacctgggcttcgactcgctgacggccgtggagctgcgcaaccgcctctccgccgccaccggcctgccactgcccgccaccaccgtgttcgaccacccgaccccgctggacgcggcggcccacctgctcgacgcgctgggcgtcgcccccgcgcccgccccggccaccccggtcgtgacggccgcgcgggacgacgacccgatcgcggtcgtcgccatgggctgccgcctgcccggcggcgtgtcctccccggaggacctgtggcggctgctcgacggcggcgtcgacgccatcggcccgttcccggacgaccggggctgggacctggggtcgctgttcgacgacgaccccgacgcggtcggcaagtcctacgtgcgcgagggcgggttcctggcgggcgcgggcgggttcgacgccgcgttcttcggcatctccccccgcgaggcgctcgccatggacccgcagcagcggctgctgctggaggtggcctgggagaccgtcgagcgggccgggatcgacccgacctcgttgcgcggcgcggacgtcggcgtgttcgccggggcgggcgcgcagaactacggcagcggccccggcccggtgcccgagggcctggagggctacctgggcgtgggcggcgcgacgagcgtggtgtccggccgcgtctcctacacgctcggcctcaccgggcccgcgctgacgatcgacaccgcgtgctcctcgtcgctggtggcgatccacctggcggtgcggtcgctgcgctcgggcgagtgctcgatggccctggcgggtggggtcgcggtgatgggcgagcccgcggcgttcgtggagttctcccggcagcgcgggctcgccccggacgggcggtgcaagtcgttcggcgcggaggcggacggcacgacgtgggccgagggcgcgggactggtgctgctggagcggttgtcggtggcgcgggcgcgcgggcacgaggtgctggcggtgctgcgcgggtcggcggtcaaccaggacggggcgtccaacggcctgaccgcgccgaacggcccgtcgcaggagcgggtgatccgggcggccctggccgacgcggggatcaccccggacgcggtggacgcggtggaggcgcacggcaccggcaccaccctcggtgacccgatcgaggcgcaggccgtgctggcgacctacgggcaggaccgcgagcagccgctgtggctggggtcgctgaagtcgaacatcgggcacgcgcaggcggcggcgggcgtcgcgagcgtgatcaagtccgtgctggcgctgggccggggcgtgctgccccgctccctgcacgccagcaccccgaccccgcaggtcgactggtcctcgggggcggtggagctgctggcgcgggcgcgggagtggccggagaccgggcgtccgcgccggatcggggtgtcctcgttcggggtgagcggcaccaacgcgcacgtggtcctggagcaggcccccgagccggaacccgcgcgggaggcggaacccgcgcgggagtccgcgccagggccggagtccgttccgccgctgaccggggccacgccgtggctgctgtccgcccgctcccccgaggcgctggcggaccaggccgcccggctggtggacgccgtgcccgccgagtggcgggcctccgacgtgggctggtcgctggccaccacgcgggccccgctggagcagcgggccgtggtcgtggcgcgggacaccgcgcgcgggctcgccgccgcgtccgcgctggccgccggacgccccgacccgcacgtggtcaccgggaccgccgacgtggacggcaggaccgccttcgtcttccccggccagggcgcgcagtgggcgggcatggggcgggaactcctggacgcctcgccggtgttcgccgaacgcctgcgcgagtgcgcggcggccctgcgcccgtacaccgactgggacctggtcgaggtgatcacctcgggtggcgcgctggaggacgtggacgtcgtgcagcccaccagctgggcgatcatggtgtcgctggccgcgctgtggcgctcgctcggcgtccacccggacgcggtgatcgggcactcgcagggcgagatcgccgccgccaccgtcgcgggctggctcagcctccaggacggcgcgaagatcgtcgcgctgcgcagccagctgatcgacgagcacctgaccgggctcggcggcatgatgtccgtcgccctgcccgccgaggacatcgacctgaccggctaccagggccggttgtgggtggccgcccacaacggccccaccgcgaccgtggtcgccggggacgccgacgccctggcggagctgcgggacgcgctggagggcgaggcccgcacccgcgtgatccccgtcgactacgccagccacaccggccacgtcgacgccatccgcgaccagctcgcccggatgctcgccgacgtcaccccgcggcccggcgagatcccgtggctgtccacggtgaccggcgagtggatcacccccggcgacgacgacgccgactactggttccacaacctccgccgcaccgtccacttcgccgacgggatcaccaccctgctcgacgccgggcaccgggccttcgtcgaggtctccacgcaccccgtgctcaccccggccgtgcaggaggccgccgaggcgaacccggcgctgcgcaccgtcgccgtgggcaccctgcgccgcgcggacggcggcgcggagcgggtggtggcgggcctggccgagctgctggcgcgcggggtggccgtggacccggcggcggtgttccccggtgcgaggcgggtcgcgctgccgacgttcgcgttccggcacgagacgttctggctctcgcgggcgctgcccgacgcgcggccggtgccgcagggcgggcacccgctggccccggtggtggtgagcgatccgggcacgggcggggtgatcctgtccggccggatctccgcggccacccacccgtggctgctcgaccacgccgtcgcgggcgcggtgctgctgcccggcgcggcgctggccgagctggcggtgcgggccggcgacgagaccgggacgcccaccctggaggagctggtgatcggcaggccggtggtgctgcccgaggacggggagctgcggctccaggtggtcgtgggcgccgaggacggggcgcgccgcgaggtgcgcgcctactcccgcgccgacgacgccgcgccgtggaccgagcacgcgagcggcacgctgtcggcgaagtcctcgctgcccgccgacgtcccggccgccccgtggccgcccgcgggcgcggagccgatcgcgctggacgggttctacgaggccatggcaggggccggttacgggtacgggcccgcgttccgggggctgcgcgcggcctggcgcgacggggacgacgtggtcgccgaggtggccgtgccgcgggcgcaggagcaggtggcgggccggttcggcatccacccggcgctgctggacgccgccctgcacgccgggaacttctgcttccccgcgcaggacggcgagcgggccacgatgctgccgttcagctgggacgacgtgcggttgcacgccaccggcgcgacgtcggtgcgggtgcgggcccgcgcggtgggcggccctggcgcgcccgcgctgaccgtggcgatcaccgacccgagcggggtgccggtggccggggtgggcgcgctcgggatgcgcgcggtcagccccgagcagctgggcgcgccgggcgtcggcggtgacgcgctgcgggtgctggagtgggccgaggtggcggtcgaggcggcggaccggtgggccgtgctgggctccgagcggcacccggacgtggacgcctacgcggccgacccggaccggccgggggcgctgctggtggacgtgggcgcctggctgggcggcgacgacgccgtggcccgcgcgcacgcgctgaccagcgcggcgctggagctggtgcgggactgggcgacccgcggggacctgggcggtgagcggctggtgctggtcacgaccggggccgaggacgtgcgcgacaccgcgccccgcgacccggcgcaggccgccgtgtggggcctggcgcgctcggcccgctcggagcacccggaccggttcgcgctggtcgacgcggacgaccggtccccggcgacgctcgccctggcggccgggtcggcgttcccggaggtggtcctgcgcggcgagcgggcgcacgcgccgaggctggcgcgggccgtccccggcaggccggtggcgctggacccggacggcacggccctgatcaccggcggcaccggcgccctgggcgcgctcgccgcccggcacctggtgaccgcgcacggcgtgcggcgcctgctgctcaccggccgccgggggccggacgcccccggcgcggcggagctggccgaggagctgcgcgggctgggcgcggacgtgcgggtggaggcgtgcgacgtcgccgaccgggacgcgctcgccgcgctgctcgcgtcgatccccgccgggcgcccgctcaccgccgtcgtgcacgcggcgggcgcgctcgacgacgccccggtgaccgacctgaccccggagcggctgtccgccgtgctggccccgaaggtcgacgcgctggccaacctggacgagctggtcggggacgggcccgcggtgttcgcggtctactcctcggcgtccggggtgctcggcacggccgggcaggcggcgtacgcggcggccaacaccttcgcggacgcgctggtgcgccgacgccgggccgagggccgggcgggcgtgtcgctggcgtggggcctgtgggcaggcgccagcgagctgaccggcgacctggccggtgaccggctcgcccgcacccgccggggcgggctggtgccgctgaccgccgccgagggcatggcgctgttcgacgcgggcgcggtcaccacgggcggcccggcgctggtcgtgccgctgccgctggacctggcggcgctgcgcgcctccgcgcgcgacgaggcggtgcccgcgctgctgcgcgcgctcgtccccgccgcgcggcgctcgctctcccccgccaccgggcaggccgcgcccccggccgggttgcgggcgcgcctggccgggctgtcgggcgacgagcaggaggccgtgctcaccgagctggtccgcgacctggccgccgccgtgctcgggcacggcgagaagggcgcggtgggcccggacgacgcgttcttcgagatcggcttcgactccatgacggccgtgcagctgcggaaccggctgaacaccgccaccgggctgcgcctgcccgccgcgctgctgttcgaccagccgacgcccgcgatcgccgccgaggcgctgcgcgagcgactggccgccgagcaatcgggctcagggcaatcgggcgcagggcagccgggcgcagggcattcaggcgcagggcagtcgagcgcagggcgatcaggcgcagggcagtccaccgacccgaccgacgagaggtgagcaccagcatgatcgacgtggccgagtacctgcggcgcatcggcgtggagggcggcgtgccgagcccgacgctggagtcgttgcgggcgctgcacaagcggcacctgatgtccgtgccctacgacaacggcggcgcggccgaccggttgccgccgaaccgggggctcgcggagatcccgctgccccgtgtgttcgcgcacgtggtgaccggccgcaacggcggggtctgctacgagctcaaccggctcttccacgccctgctcaccgcgctgggctacgaggtgctgatggtcgcggcggcgatccggctggccgacgaccggttcgggccggacgaggagcactcgttcaacctggtgcgcctggacgggcggacctggctggtggacgtggggttcgtcggcccgtcctacctggagccgctggagctgtcggcggtcgagcaggagcagtacggctgcgcctaccgggtcgtggagcgcggggacgcgcacgtggtggagcgcaggcccagggacggggcgtggcaggcggtgtaccggttccggccggggcgggcggaccgggacggctgggaggcggtgcggttggacgggctggacgactacgcgcgggactcggtgctggcgggcaccacgttccggggtcgggcggcggagaacgggcagcacgtgctgatcggccgccgctacttcaccgtgctggacggggtggagacgacgcgggtgctcgtgaagaaggacgagttcgcccgcgtcaccgagtcgatcatgatcggggggtgagcgcgtggcgggcgaggtcgagcacgacgtggtggtcgtcggctacgggccggtggggcagctgctgtcggtgctgctggcgcagcgcggctggcgggtgctggtgctggagcgctggccgacgccgttccggctgccgcgcgcggtcgggttcgacagcgaggcgacccgcgtgctggcctcggccgggctcgggcccgcgctggccgagttcggggagcccgcgggcgactacgagtggcgcaccgcgtccggggagacgctgatcgcgttcaccgtgcgggaggaggggcactgcggctggcccgaggcgacctcggcctaccagcccgcgctggaggacgcgctgatcgcgcgcggcgaggcgctgccgggggttcaggtgcggcgcggctgggaggtgaccgggctgaccgaccggggcgaccacgtgcgggtggtggccaccgaccccggcggggcgcgcgtgaggctgacggcgcggttcgcggtcggctgcgacggggcgaacagcgtggtgcgggcccgcaccggcaccgacgtgaccgacctggacttctcgcacgactggctggtgtgcgacgtgcggctgcacgaccggcgcccggtgacgccgaacaacctccaggtgtgcgacccggccaggccacgcaccgcggtgtcggcggggccagggcaccggcggtacgagttcatgcgggtgcccggcgacgacccggagcggttcggcacgccggagagggcgtgggagctgctggcgctgttcggcgtcgggcgcggcgacggggtgctggaccggctggccgtgtacacgttccaggcgcggtgggcgcggcggtggcgggcgggccggatgctgctggccggggacgccgcgcacctgatgccgccgttcgccgggcagggcatgacctccgggttccgggacgcggcgaacctggcgtggaagctggacctggtgctgcgcggcgaggccgggtcggcgctgctggacagctacacgctggagcgcgccgagcacgtgcggcacgccgtgacgatctcggtgggcctggggcgggtggtgtgcgtggccgacccggcggtggctgcggaccgggacgcggcgatgctggcggcgcgcgagcgcgagctgacaccgggcgcgtcggcccggtcggtgctcaagcccctggaggacggggtgctgcaccgggacggcgacggcgccctcgcgccgcacgcgggggccgtgggcccgcagtggcgggtggggcgcggcgggcgggtcgggctgttcgacgacgtggtggggaccgggttcgcgctgctcaccaggggcgggctggtggcggggccggaggtgcgggcgcggctggacgggctgggcgcgcgctacgcgcacctggtgcccgccggggcggcggcggacgggccggacgacgtggtcgacgtgagcgggaactacctgacgtggctggaggagctggacgcggcggcggtgctgctgcgaccggacttctacgtgttcggcgcggccggggacgcggcggggttggccgggctggtggcggacctgcgcgcgcggttggggtgacgccccgcaggccccggcacgtgccgcgccggggcctgctcgcgcgtcacgtccggtcgtcggcgaggtgggccaggcaccagtcgagcacctgcgagggcttgcggaccaccgcgtccgggttcgccgccagcagctccgcctcgtccgtctcgccccacagcgcggccagcgccgggtagcccgcggcgcgggcgctggccaggtccgtcagggcgtcgcccaccatcaccacccggtcggccgggacgtcgagcaggccggtggccagcaggagcatgtccggcgcgggcttggggttcgcgacctcgtcggagccgatgatatggtcgaacagccccgccatgccgagggtggtcagcagcgaccgggcgcgcggcccgctcttgccggtgaccacggcggtgccgaagccgtgctgccgcaggtccgccagcagctccggcgcgccctcgaacacctccacctcacccgccagccggtagctctcgcggacgaacggaccctccatctccagcggcaggtccatgatccgcatgatgtccgggaagtaccgccccaggtgccggttgtactcctcgaacggcgcgggcccgtcgccgacgacctcggcgtaggcgatctcgaacgcctgccgcatgacggcgaagctgttgaccagcaccccgtcgaggtcgaacagcacggcccggtcgtaggtcgcgccggggacgtgccggtggggcgcgggggtcggcggggcgagggggcgcggggccgcgggcgccggaaccgcggtcgcggcccgctcgtccccggctcgggcccgcacgactcgggggttggtctgtccggtggtcatcacggggctcccgtcgggacgaggtcgaccggcgcgtgtcgtcgttcggcgcaccgcacggtgtcggcggcgcggtagacccgttcgatcgcgcccgcgatccagcgcgccccggacgccgcctcgccgcgcgtggcggggtcggccaggcgggtgggcaggctcgcgagctgggcgtcgtactcggcgcccaccggctcggcgggcagctcgaccggggtggtgcggccgtcggtggtgagcaggagccgcgacgggccctcgcggttcgggctgaagccgaaggtgcagcgcagctccgccgtgccgccgctgccctccacgcgcacgaccgtggtgtccagcgcctggtgcgaggcccaggccgcgcgcaccccgatcgagatccccgaccgggtgacgaggaaccccctggcggtgtcctccacgtcgccgaccaccgggtccaccggcgccccgtcgccgcgccaggcggcccggaacgcgtggtcgttgacgaagtccgcggacaccgcgccggtgacgtgctccagctccgcgccctccgggtcgccggtggcgccgcgcagcagcacgcgggcggtgtcgagcaggtgccagccgaggtcgaccagcgcgccgccgccggagcgggtgcggttggtgaaccagccgccccggtccgggatgcccttggaccgcacccaggacacgtcgacgtgccgcagcgcgcccagcgacgcggccacctggcgcagcgcccgcacgtcggcccggtgccgggcggcgctcccgcccagcagcaccgcgccaccggcctgctcggcggcggtgagcgcggcggcctcggcggaccccaggcacagcggcttctccaggaacaccgggacgccccgccgcagcagaccggacgcgaccggcgcgtgcaggtggttcggcacggcgaccacggccaggtcgacctcgtcgcggcgcaggtcctccacccgctccagcgcggtgatcccgcgggagccgggcacggcggcgcgggcctgcgcggacggctcgaccacggcgacgacccggaaggcggggctgcccagcaaccggggcagccacacctcccgcgccacccacccgagcccgaccaccgcgacccgcaccggaccaccgctcccggccctcggcccgtcgctcacaccaccacccccgctccccgcgcccgccaccccgcgctcacgcgcccgcgaccacgtcggccacgacggcggcaagccggtgcagctgctcctcggtgcccagcagcacccggtggtgcagccacacgcagtcgcgggtgatctcctccgacaccgggcaccgggcggccagctcctcggtggtcaggtcgggcgcgccggtctcccagaacgcctgggtgcggtagaccgcccggaacgccatgaacgccgggatcccgcgccgcaccagctcgtccaccaccgcgttgcgccgctcctcggtgacgccgggcatccggaacatcgccatgtagctcgggttgcggtcgctgcgcgggtcgacggtctgcggcacgacgccgtcgatccccgccagcagcgcggacagcaccggccagcgggcctgcctggtcgcgatctgcgagtccagcctgccgagctgggcgcgcagcacggcggcggagaactcgttcatccggaagttcgagcccgaggtgaggtggaagtagccgcggtcgcccttgggcctgccgcagctgtgcaggacgaacgccttctcccactgggcctcgtcctcgaacagcacggccccgccctcgcccgccgtcatcagcttgccgttctggaagctgaacgtggcgatcgacccgagctcgccgacccgcttgccgcgccagtgggcgccgtgggcgtgggcggcgtcctgcaggaccggcacgccggtgctcgtggacagcttgtccagccggtccatgtcggcgaactggcccgccatgtgcacgggcatgatcgccgaggtgcgggaggtgacggcggcctcggcggcggcgacgtccaggcagtaggtgtcggggtcgacgtccacgggcacggcgaccgcgccgaggcgctgcacggcctgcgaggacgagatgaaggtgaaggcgggcacgatcacctcggcgccggggcccacgtcgagcacctggagcgccagctccagcgcgtgcgtcccgttggtgacggcgagcgcgtggcccgcgccgtggtactcggcgaactcgcgctcgaactcgtcgacctcgctgccgccgacccgccaccactggccctggtccagcgcgcgcagcagggccgtgcgctcggcgtcgtcgtgctgcggccaggccgggaactcgatgcctgcgtccggagaattgctcatgagcccctgtcccgtcgttcgcggaaatggcgcgggggaattcgccgcggcctgctttcggaattcgacgctaccgattccgcagatcccgaccaacccccttgacctccccctaatcccccctgttcccaggccatcaccgcagcacgcgggcacagcggcacagccgtgcgcacaatgggggcgaacgggaaccggggcgtccgcgcgccccggcggcgctttcggggaaaggtgtcaggcgtgggcgagctgctgctggtgaacgggccgaacctcggcatcctggggcgccgcgaggtgtcggtgtacgggaccgacacgctcgcggacgtcgagaaggcggtcggcgaggaggtcgccgggcgcggctggtcggtccgctcggtgcagcgcaacggcgagggccagctcgtggacgagatcgaggcgtcctacgacacggtgggcgcgatcgtgaaccccggcgcgctgatgatggcgggctggagcctgcgggacgcgctggcgaactacccgcgcccgtggatcgaggtgcacctgtcgaacgtgtgggcgcgcgagagcttccggcacgagtcggtgctggcgccgctggcgagcggtctcatcgcgggcctgggcgcgcgcggctaccggttggccgcccgcgcgctgctggacctggtggactgaccgccgtcgcgcgcgagcccggccgcgtgcacggccccgcgcagcgaggacaggccgccgagcagcgcgggccgcacgggcgcggtcgggtggccgggccgggcgagtgcggcgcagcggtcggcgaccgcgtcgacgtatccgggcacggcggcggcgaacccgcccccgaccacgcacagcgagggccgcgccagctcgccgacgccgacgagcgcggcggccagcgcccgggccccccgctcgaccgcggcccgcgcccacccgcgcccgtcgccgagcgcccgcaccaggtcctccccggtgaccggcgcgccgccgagcctggcggcctcggcgagcacggccggtccggacgcgaacgcctgcacgcacccggcccgcccgcacgggcagggcggcccgtcgagcgccaccacgacgtgccccagctcgcaggacccgcgctcgggtccggggaacggcaggccaccggacacgacgcccccgccgacgccggtccccacgcccgcgtagaccaggtcggcgcacccgtgggcacgggcctcggcgagcgcggcgaggtcgccgtcgtccgcgacgagcaccggggcggccagcccgccgaggaaccccgcgaggtcgacgccgacccaccccggacggctgggccaggccgtgacgaccccgccgtcgacggtgccggggaacgcgatcccgaccccgtcgagcggcgccccggcccgcccggcgaggtcggcgacggcgcggccgagcaggtcgaggtcggcgcgcggatcaccgtccccaggccaccggaagccctcccgcagcaccagcggcccgcgttcgagccgcagcgccaccttggtcccgccgacgtccaccccgagcagcgccccgctcacaccccgacctcccgccgtccgcacccctcgccgcaccaccacccgcgcgggccgccgcccacgacgccgcccgccacaccgatacccgcgcggtcctcgctcacgacgccgccacaccaccaccgcacgggcctgcggtcacgacgccgccacacctccacccagcgcaccaccacccgcgcgggccgccgctcacgaggccaccgctcacgacgccgccgcccgccaggccgccaccgcctcggcccgccgggcgtcgccgctctccaccagcgcgaaccggatcgcccgcgtggccgcgttcgccgcctgcagcgcctcgtgcgcgccgggctcggggtcgctgcgcccggtcgccacctcggtgatgctgcgcgccgcctccaggcacgccaggcaggccgcgacgtacgcgtccgccccgcctcccgcgccgcccagcttctccagcagccgcgtcacgtcggcgtgcacctcgcgcacggcgtcctccacccggcccgcgccgggtggtgtgccggggatgggggtcaccgctgcctcccggtgcctgacgcggacccggtcagccgagggccgggatgagttcgacgaaccgaccctgccacaaccggcgcagctcgcgcgtcaactcctcggacggcgcgccgccgaccagctccgccagggtcgtcacgccgtccacccggctgagcagcgcgtgcgcctgcgcggtggtggcggtgacgggcccgtggtcgtactccagcgacacctcgtgcaccggctcgcccgccgccgcgctgcccggcgcgggcgcggtgcgcacggccaggcgggtcaccgggcgcagcctgggcaccagcgcgcccaggtcctgctcggggcgggcccgcaccaggaagccctccacgaccaggccgtccaggtcggtggtcaggaagctggtgagcacgtcgtccaggctctgcgcgatcggctcggcgttgttgttgaacgaggtgttgagcagcaccggggtgccggtcagctcgccgaaccgcgccaccagccggtggaagcgctctcccgactcgggcgtgacgacctgcacgcgcgcgctgccgtccacgtgcgtgaccgcgcccagctcggcccgcctggcgggcagcaccggcaccacgaacgacatgaactcgtggtgccccagcgccccggacaggtcgaaccagtcccgcgcggcctcggcggtgaccacgggcgcgaacggccggaagctctcccgcttcttcaccatggcgttgatccgcgtctggttctccgcgggccgggcgtcggcgatgatgctgcggtgcccgagcgcgcgcggcccgaactcggagcggccgtgcgcccagcccagcacctcgccgtcggcgagcagcttcgccgcggtctccaccgggtccaccagcggcgtcacctccaccaccggcgaccagtccgcgagccgcgcggcgacctgctcgtccgtgccgaggtccggcccgagcgccgccgacaccagccgcgccgacggccgctccagcacgcccagcgcggcggctgcggcgtacgcggcgccctcgccggcgcccgcgtcgtgcgaggcggggtggatgaacacctcgtcgaacagcccggccttgaggatgcggccgttgagcgtggagttgtgggcgacgccgccgccgaacgcgagcgtgcgcagcccggtgacctcggcccagtgcccgagcacgtgcagcgcgatcttctcggtcgcctcctgcagcgcggcggcgaagtcccggtgcgcctggctgaacggctcgtccttgcggcgcggccggaacccggcggcgaccagcgcgggggtgaccaggttcggcaccttggtgttgccgatcaggtcgtactcgcccttgtcgcgcagcgcgtgcagcccggagaagacctcgcggtaggtcgacgggtcgccggacggggcgagccccatgaccttgtactcgtcgccgaagccgtagccgagcaggaacgtggcgttcaggtacagcccgccgagggacttctccaccgggtagtcgtgcagcttctccaggtgcgcgccacgcgcgtggtagaccgtgccggagttgtcctcgccccggccgtcgaagatcaccaccagcgcctcgtccgcgcccgagtgcaggtaggacgagtaggcgtgcgcctcgtggtgcggaacgtagacgagcttgtcgtcgggcagctcccagccgaggtcctcgcgcagccgctgcttgatcagctcgcgggagaaccgcagcggcaccctcgggtgctcggtgtagacgtggttgagcaccaggtcgaggtggtcctcggggaagtagtagccgaccgcgtcgacgtcgtccacggtcgcgcccgcgagcgccaggcactcgcggatggcggtggacgggaacttggtcgtcttcttgacgcggttgagccgttcctcctccacggcggccacgagctcgccgtcgcgcaccagggacgctgccgcgtcgtggaagaacagctccgacatcgacgggacgaggtcggtctcggcgggcgagaagttcccgttgatcccgagcacgagcatgtggcatcaccttgatccgggaggcgagggctggggcgcggagggggtcgccgtcaggcgcggggcgcgacggcggcgaggtcgggcgaggtcagccgcagcgcggtgggcgcgggcttcgcggtcggcacgaggtggaagcgctccacgccctcctcggtgggcgcgggcagctcggcggcgcaggcgcagtcggcgggggcgaacccggcgaaccggtaggcgatctccatcatccggttgcggtcggtgcgccggaagtcggcgaccaggtgcgcgcccgcgcgcgccgcctggtcggtgagccaggtcagcagcgtcgacccggcgccgagcgagacgacgcggcacgaggtggccagcagcttgaggtgccacacccgccggcgccgctccagcagcacgatgccgaccgcgccgtgcggcccgaaccggtcggacatggccacgaccagcacctcgtgcgcggggtcggcgagcaggccgcgcagcgcccggtcgtcgtaatgcacgccggtggcgttcatctggcttgtgcgcagggtcagctcctcgacgcgggtcaggtccgcctcgcccgcgcgcgcgacgaccacctccaggtccagggtgcgcaggaactcctcgtcgggcccggtgaagccctcgcggctggcgtcgcgctcgaagcctgcgcggtacatcagccgccgctgccgcgagtcggcggtgaccacggcggggctgaactcggggcgctcgggcagggaggcgacgtcggcctcggtgtacaggcgcacctcgggcagggcgcgggccacctcggcgcgctcgacggggctgtcgtcgacgaacgcgatcgtgcggtgggcgaagccgagccggtcggcgatggcgcgcaccgacgccgacttggcgccccagccgatctgcggcagcacgaagtagtcggcaaggcccaggcgttcgagcacgggccaggcgtggtcgtggtcgttgcggctggccacggactgcaggacgccgcgcccgtcgagcgcggtgatcacctcgcggacccgctcgaacgggacgacgtcggcgtcctccaggagggtgccgcgccacagggtgttgtccaggtcccagaccaggcacttgaccgtcggtgcgggggtctcggtcacggctgctgctccctgagcggagttcggctgcgctggtcaagtccgcgcggggcccggctgcgcacgtgctgggcgagcacgagctggcagatctcggaggtgccctcgatgatctccatgagcttcgcgtcccggtgcgcccgcgccaccacgtgcccgtcgctggcgcccgcggagccgagcagctgcaccgcgcgcccggacccggcggcggcctcgcgcgaggccaggtacttggcctgcaccgccgccaccgccaggtccggcgagttcgcgtcccacagcgcgctggcgtgctcgctcgcgcgggcggcgacctgctcgccgacgtgcaactcggccaggtgccgggcgacgagctggtggtcggccagcacgccgccgccctgctcgcgggtcgtggtgtgctcgacggcggcggccaggcaggcgcgcaggatgccgacgcagccccacgccaccgacacccgcccgtaggtcagcgcggcggtgaccaccagcggcagcggcagcccggtgccgcccaggacgtcggcggcgggcacccgcaccccgtccagggtgatcccggagtgcccggcggcccggcacccgctggggttcggcaccctctccacccgcacgccgggcgcgtcggcgggcacgacgaccgcgctcgccccgccccggtagtgcccgaagaccaccagcaggtccgcgtagtgggcggcggtgatccacgacttgcggccggtcaccaccacctcgccgtcgccggtgtcggtgatggtcgtggtcatcgcggacaggtcgctgcccgcgcccggctcgctgaacccgaccgccgccagcccgcccgaggtgagcctgcgcaggaaccgctcgcgctgctcggcggtcccgagcctgcgcgcggtccacgccgccatgccctgggaggtcatgacgctgcgcagcgagccgcacagctcccccaccgacgcggtcagctcgccgttctcccggctgcccagcccgaggccgccgtgcggggcgccgacctgggcgcacagcacccccagcccgcccagctccaccagcagctcgcgcggcagctcgcccgccaggtcccacccggcggcgcggtcgccgacgcgctcggcgaccagcccggccagtgcgacggcgtcgctcaccgccccgcctcccgcagccgcagcaccagcgtggtcatggtgttgacggtgcggaagctgtccaggcccaggtccggcccgtcgatcacgacgtcgaaggtcgactccaggtgcaccacgagctccatcgcgaacatcgaggtgacggtgccggacgcgaacaggtcggtgtccggctcccaggtctgcttggtgcgctcggcgaggaacgcctgcacccgctcggccaccgcgtcggcggtgagcgcgccgggctgggaggaggtcgtcacagctgtgccttcccgtagtcgtagaagccccgcccggacttgcgcccgaggtgcccgtcgcggaccttgcgcagcagcagctcgcagggcgcggagcgggggtcgccggtgcgctcggccagcacgcgcagcgagtcggccaggttgtccaggccgatcaggtcggccgtgagcagcggtcccgtgcggtggccgaggcagtcctgcatgagggcgtccacggcctcgacggaggccgtgccctcctggacgacgcggatcgcgtcgttgatcatcgggtggacgatccggctggtcacgaagccggggccgtcgccgacgacgaccggtgtgcgggccagctcgcccagcacgcccacgagggtctccagcgcgtccgcgccggtgcgcgcgccccggacgacctcgaccgtggggatcaggtacggcgggttcatgaagtgcgtgccgatcagccgcgccgggtcggggacgtgcccggccagctcgtcgatcgggatcgaggaggtgttggacaccagcggcacgcgcggcccggtgagcgcggcggccccggccagcacctcggccttgaccggcagctcctcggtgaccgcctccaccaccagcgagacgtccgcgacgtcggcgagcgaggtggtggtgagcagctcgccccgctcgcggtcctcgggcagcgcccgcatcagcctggccatgcgcagctgggcggccaccgcctcccgcgcccgcccgaccttggcccggtcggtctcgaccagcaccaccggcacgccgtgcccgacggccagggaggtgatccccaggcccatcgtgcccgcgccgagaacggcgagcaccgtcctgccgtcctgctctcccatcgcgctcccccgccgcggccaccgcggccgccgtccggtccgcgcgccgtcccggcacgcgcattccaccctcgatcgtgtgccgggaaaggcgcgcccgaccccctgacctgcccccctgaacccccctcaacggaaccggaaatcgaatgtcccgaacgcgccgtcaaatcgtcgattgacagccgcagaactgttcatagactgtggcggcagtaccgatctccgaattccacggaagagtcctcccccatggctcagcagatcagcgccacctcggaaatcctcgactacgtccgcgcgacctcgttgcgcgacgacgacgtgctcgccggtctgcgggagcggaccgcggttctcccggccgcgtccgcgctgcaggtggccccggaggaggggcagctgctcggcctgctggtgcgcctggtcggcgcgcgctcggtgctggaggtcggcacctacaccgggtacagcacgctgtgcatggcccgcgccctcccgcccggcggacgtgtcgtgacctgcgacgtcgtcgcgaagtggccggacatgggcaggccgttctgggagcgggcgggcgtcgcggaccgcatcgacgtccgcgtcggcgacgcccgcgccaccctggccggcctgcacgccgagcacgccgtgttcgacctggtgttcatcgacgcgaacaagtcggattacgtccactactacgagcgcgcgctgacgctgctgcgcaccggcggcctggtcgtcgtggacaacacgctctttttcgggcgggtcgccgatccgtccgcgaccgatccggacaccaccgccgtgcgcgagctgaacgcgctgctgcacgccgacgagcgggtcgacatgtgcctgctgccgatcgcggacggaatcacgctcgccgtgaagcggtgaacccgcccgaatcgcgccgaattcccccggagagaaaggccgccgcagtgttcaccgaggacgtggccaccgacctgcccgcctacccgttcctgcgggaccggggcgactgcccgttcgcgccacccccgcgctacggccaattacgggaggagcagcccgtcaccagggtccgcctgtgggacggcagcaccccgttcctgctcaccggtcacgaggtgtgccgcaccgccctgaccgacccgcgcttcagctccgacggcgccaaccgcgcccagccgcgcttcgtgaagttcgacatcccggacgacgtgttcaacttcggcaagatggacgacccggagcacgcgaggctgcgccgcatggtcgccgggcacttcgcgagccgccccgtggaggcgatgcgccccgcgatcaccacgatctgccacgcccagctgcgccagctcgtgcaggcgggctcccccgccgacctggtggcccactacgcgttcccgatcccgtccctggtgatcggcggcgtgctcggcgtggcgggccccggcctggacgagttcgcgcgcgactcgacgcgcgccctggacccgtccctgtccgccgaggagatgggcgccgccatcaactcgatggtcgggttcgtggacgacctgtgcgcggccaagcgggccgcccccggcgacgacctgatcagccgcctggtgctggacttcgagcgcaccggcgagctgacccggaagcagctcgtcgccaccgtgatggtcgtgctgctggcgggctacgagaccaccgcgaacatgatcgcgctgggcacgaccgcgctcctgcgcgaccccgagcagctggccttcctgcgcgccgagcccgccggtttcgccaacgccgtcgaggagctgctgcgctggcacaccatcgtccaggacggcaccggccgcgtggccctggacgacgtcgagctggacggcgtgctcgttcccgcgggctccggcgtgatcgtcaacctgcccgcggccaaccgcgaccccgacgtcttccccgatcccgaccgcctcgacgtgaccaggcacaacgcccggcggcacttcgcgttcggctacggcgtccaccagtgcgtgggcatgacgctggcgcgcgtcgagctgcagatcgcgctggagaccctgctgtgcggcctgccgggcctggcgcctgccacgccgttcgaggacctggacttcgccctggagtccatgaacctcggcctgcgctcgctgccggtcacgtggtgagcaccgaccgtccaccaggggagagccgatgacccgcaccacccccacccccgacctggccccggagttcccgatgcccaggtcgcccgagcacccgttcgacccgccccctcgactccgcgaggcgcaggaggcgggcggcctgtcgcgggtgcgcctgtgggacggcagcaccccgtggctgatcaccaagcacgcccaccagcgcgagctgctgcgcgacccccgcctcagcgcggacttcctgcgccctggctaccccagcccgattcgcatcgaggacaagtcgacgttcatcagcagcttcccgctcatggacgaccccgagcacaaccggcagcgccggatggtcctgggcccgttcaccgtccgcaaggtggaacgcctgcgcccgttcgtgcagcggatcgtcgacgagaagatcgacgaactcctcgcgggccccaacccggtcgacctggtcaccgcgttcgcgctgcccatcccgtccctcgcgatcagcgccgtcctgggcctgccctactccgaccacgaggtcttcgagcgcaacagcgccgtgctgatccgccaggacgtgcccccgcaggaacgggccgaggccagcgaggagctccagcaccacctcgaccgcgtcctgggcgacaagatgaccgaccccgccgacgacctcctctccgacctgggcgcacgggtgctggcaggcgagatcagcaggccggaggcggtcgacatgaccgtcctggtgctggcgggcgggcacgagaccaccgcgaacatgatcgcgctcggcaccctcgcgctgctccggcaccccgaccagctggcgctgctccaggcgggcgacgaccccgccctcgccgagaccgccgtcgaggagctgatgcgctacctgacgatctcgcacaccgggatgcgccgcgtggcgaccgaggacgtggagatcgacggccaggtgatccgcgcgggcgagggcgtggtgctggcgacctcgatcggcaaccgcgaccccgacgtctacgacggcgacccgcacgtgctggacctgcgcaggccggtgaagcagcacttcgcgttcagcttcggcacccaccagtgcctgggccagtcgctggcccgcatggagctgcaggtcgtcgtgaacaccctctaccgccgcgtcccgaccttgcgactggcgaccgcgctggagcgcatcccgttcaagcacgacgggatcgtctacggcgtctacgagctgcccgtcacctggtgaccccgtcccaccagacctcctgccacgcagacctcccgcaagccgaccccgaaaggccgttcccatgagcgacaccacgctgtccgtgcccgtccccgaggaggtcggcaagctctacgaccagatcctgaaggacgagcacacctacgagcagttcgagaagttcaaccaccagctgcacatcggctactgggacgacccgacctcggacgtgcccatgcgcgaggccgtggtgcgcctgaccgagctgatggtcgagcgcctgcgggtggacgccgaggaccgcgtgctggacctgggctgcggcatcggcggcccggcgacccagatcgtgcgcaccaccggcgcacgcgtcgtcggcgtgagcatcagcgaggagcaggtcaagctcgccaccaggctggccaccgaggcgggcgtgggcgaccgcgccaccttccagcgcgccgacgccatgcggctgccgttcgaggacgagtccttcgacgcggtgatggccctggagtcgatcctgcacatgccgtccagggagcaggtcctgtccgaggcgcgccgggtcctgcgccccggaggccgcctggtcctcaccgacttcttcgaacgcgcaccccgcacgccggggatgcaccccgcgatcgagggcttctgccgaaccgcgatgacgacgatggccgacgtggacgactacgtgccgatgctgcaccgggtgggcctgcgcgtgcgggagctgctggacatcaccgagcagaccatggaacgcacttggcgggagaccctggagatcgtcagccagaacgaccgcccggtcgacttcgacctggcggagctgttcggcgtggacgagttcggctgcctgctggtcgccgcagaccgcccgtgaggcccgtccccgaggccgtgggccgcctgtacgacgacctgctggaggccgagctggaggggggcgcagccgacccgaacctgcacatcggctactgggacgcgccggactcgccaacgccacgcgcggaggcggtagtgcgcttcaccgacgaacacgtccgccgcctgcacgtgaccacgggcgaccgagtgctggacgtgggctgcggcgtaggcggcccagccctgcgcgcggtggacctgaccggcgcccacgtgaccggaatcagcatcagcgccgcccagatcacccacgcgacccacctggccaagtccgcgggccacgcggacaacaccaagttcctccacgcagacgcgatggccctcccgttcccggactcctcgttcgacgcggtcatggcgatcgagtccctgatccacatgcccgaccgcgagcgggtcctgaacgaggcaagacgcgtactgcgcccaggcgggcgactggtcctcaccgaactgttcgaacgcgccccaagacccacccgcagacacccagcgataaccgagttctgccgagcatcgatggtgtccctgcccaacgcagacgactaccccgcactactacaccgagcaggcctacgcctacgggaactcctggacatcaccgaccacaccgtccaacgcaacttccgcgaactggccgatctggtaggcgacgcgaagggcctgctgttccacccacgcgacctggtgggcgtcccagaattcggctgcttcctagcagtagccgaacacccgtaaccacgcggtggcgtcccccacggacgccaccgcctcgcgggctgcggggcgagcgcagcgagcccgcgcagccccactcccgcgtccctcttctccgtgtggcctggcgcatgtcaaattcccactgactgccaacagatcatgtgccgtttgagcaggtcagcgacttgtcgcgcttcggtgccttaaggccgagctgggatgggggcactgtttccggactgagcggggcagcttggaaggtggagttcggtgagcagaggcagcacgtcccgtcgcacgtagaggtggttgtacacgcggtggcgggacctgcgcagtaggccgctatccgcaagctgctccaagatcaggagtgcggcgcggtgcgtatagccgagttcggcggtcagcatggtgctgttgagcagtggggcgacgagcagcggggcgggaagcgctttgaccttcctccgcccggtgcgcatcgcccaggtgggcgatcgcgcgagcctcacggatcgcggtcacctcatgcaggctggcgctcaacctggaacgcgcgactgtttcgtccagacgtgccagggcggtgtaggcgtgcaacaaggtcttgctggtttcggagcgcagtctgagccgggaccaggacgacaactccgcgatcctcgcggacgggggcggcctcgtgtcttcaccggtggtagttgacctgcgcggggcggaggtgccctattgctgccgggacgaggtcatcccccggagcagtttctcagcacgccgtgaatcgagatccggggcgctgagcgcggtgaacgcctcgtccagcgagtcgcacgcgcacgtcgtcctgacatcgggccgcgcatggcccgaggtggtcagcggtgagcgggaaggcgcggcagggtgtgtgcgagacactccgggactccgtgcagaaggtcgatcaggcgaaagggttgaactgcgaatcgcaaagcggcccggccgcaaaggggtcgggccgcctgcgacgattggtcacgctgctgcggcgcggtcccgccggaactgcttgccgagcaggtcgatccgccccttgtgatcttctgccagcgcctccagaaccgagagcagtcgtcgggcgtgcagtgcatggccaataccatcgtcgcgtaccccagagggtgtcgctcccgttcaggggcgaccatttcccacgcccgcttggcctccttggcggcccggccaagatcgccgagcatcaggtaggtgcccgacaacccgacaaccctgcctgccaacgcggcttccggcaccccgcgcgcctcgtcggcttccaacgcccgaacaccgtgccacagcacggcccgcgcgttgccctcgctcgtctccagccatcccatgacaccgtgcgcttcggccagtgaccacgatcggctgtcgggatcggtgttgcacaacgccagctccagcgctcgttcagcagcgttaccgaccacagcgcggcgccgatgtccagcacttcttgccggtacccgcccacgagtgcggcggtgcgctgcacggccacgacttcccgccgatgcacgatcagccacttgtacgccgccaaggcgttgtcgaacagcggcttcgccccgacctcgaagccgtcgacgaacacctcgcgcaaatcaccgagcagtttccctgcggccaaggtgcgccgatgcaggtacctgcccaagcgctccaactcttgcgggaactcctgctgcacagcccatcgcagcagtgcctgggcttggtctgtctcctgcgcgcgatggcgaccggccagccggtaacgcgaggaggtgaactcggggtgcgtgatgttcgggtgaagttcagtccacgaaggctgcgtcagcaccagcacgccctggttcacccagtcccgcgcgtgttgggcggtctcctcgacggtgattcccagcatcgcggccaacgacgaggtcgagaccacggggtccggcagcaggtccagcaatctcagctcttgcggaatgggcacaagagtgttgatcatcgatgcccctcccggaggacggcgatgattggagtggcgaacagaaggggaaacgccagttcgccgggttccggcggtccacgcgccttcggccggccacttggactccgacgggcagaagttcaccggcaaggactctggtgacggtggagcggtgcacgcccatcgcttcggccaaggcgtagtcgctgtggtaaccagcgaactgagctagctttcgcatcttgtcgccgcgcaccccgacgaccttcttgatcttttcggtctcgctgtcgttgtcgtcgacatgtccgccgtccggcgctgacaccgttctccttgagatcgccgagctgaatgggggatgcttcgacgtaaggcgttgcgtatgcgcaacaggtcaggcggcgtcgagtctccccattaccgaggtttcgcttgatcgccgacggggcccgcctcgaagaagtccaatcgagctggcatccccttcgattgatcaatagcgcgacgggtgtcgctcgacatcgccccaccgcctgctcctgacgtgccacgagcagggaggagcgacctccctcgggactgcaccgaccgttcctccctgtccgccgattcagttgcattccgccacgctaggtgccggatgcgggccgaagggacaacgaagggacaagtcgaacagcccaggtgcgaggtatcttgaaatagcccgaatcctccgtcgcgaagcaggtcgccatgcccactgacgaacagtccgagggtgtcggagagcgcatagccgtccaacgcaaactggctggcttgactcagcaagctctggcgaagcgcgcacacgtcagcctcagcctcatcaaaggggtggaacagggaaggattcccgcctctcccgcgctcgtgtcccaggtctcgcgggcgctcaaggtcgaggcgacgatcttgctggggcagccgtaccgccccgaggatcggagcagtcttcgcgttcactccgtcatccccggtctgcgccgagccttggcggcctaccggttgcccgctgatgagggcatcagccctcgcgggtacgacgagctggccgccggtgtagccgccgcgtcgaagatgcgccacgccgcgacgttggacgtcctgggggctgaactccccggcctgctcgacgagatccgctcggccatcgacgaggctcggggagttgagcggcagcgcctgttcagcttgctggcagaggcatacgcagccgctggtcaagtcgcgtggaagctgggttacgcggacctgtcctccctggcgacggagcgcgtggagtgggcggccaaagagtccggcgatccgctcgcgatgggcgcagcggacttctacatcgccggtgagctgatcgcagcagcggagtggcgcggcgccctctcctacctcgacggctcccgtcgccgcctggagcacgtggtgcgcaaggacgacgaggccgccttgtcgatctacggagtcctgcacctgaagtcggggctcgcggcggcacgggccgggaaagccgacgaatccgacgcgcacctcgctgaagcccgtggcatcgcggaaagggtgccgctgggcagtgaccactaccggctcgcgttcgaccgggactcggtcaacatctggaccgtggggctggcagtggagcgcatggacggcacggaagccgtcaaacgagcccacgggatgcgcttcagcaagaccaccccgcgtgaacgcgtgggccaccactacatcgatctggcgcgcggctaccagctgcacggagaccgtgaccgcgccctgcacacccttcagatcgccaggcgaacctcaccgcagcaggtgcgctaccacccgcaggtcagggaaacccttctcacgctcgcggaacaggaccgcaggcgctcggattccctggcagggctcgcgcgctggatcggtatgccggtgtgacaggacggcgagctgacgtcgctgttgaggggcagccccccatcggccgcccctcaacagcaggtgccggtacgtccctcacagcgcgacgctgacgatcaggctggcgaacatggccacggccagcgcgatcatctgcttgggcgcgccgccgaagaagagggaccccacccaccgcagtggtaaaccggcaccgagcaccaacggccaccggccggcagagcccgtccccctcttttttggaggtctgccccgccggcgaggcgtgcccttcagctctcaagctctccactctcgatcttgtggtccgaacaccccccgcacccccactacgatccccccatgggggctctgatcatcgcggtagtgctgctgctcgtcttcctcgtgcaactcaagcgggaacccagacgactgggcaacggcgtctacctgctgatgagcctggcgttcttcgccctctggctgctcaccctcgccaccccccagaccaggacgctggtggtaggcgcggtagtcctgatcgccccggtattcgtcaccgtgatcgccctgttcctcatcgccaacggcgtcaccctgctgcgccgcgagggcgtcaaaccaggcaacgccctctccttcggcgcaggcaccgccatcctgtgcgtcgtaggcggcctgctcctggtcctgctctccgccctgcgcgaaggctcccccgacccctgggtgctggcagcagccggttccctggtcctcctggccggctacctgggcttcgccttcaccctcttcctgctctactccgtgctctacggccgagtccgcaagcgcaccggccacaccgcgatcatcgtcctgggcgcgggcgtccccggcggccgagtgaccccgctcctggcaggccgcctggaccgcgccctgaagctctaccgccgcgccgcagccaagggcgcttcccccgtggtagtcgcctctggcggccaaggcccagacgaaccagcctccgaagccgaggtcatggccaactacctccgcgaacgcggcatcccggacgaggccctcctggaagagcgcgagtccacctcgacctgggagaacctccgcctctcctccgccctgctcgccgaacgcggcgtgaccggcagactcctggtcgtcaccagcagctaccacgtcccccgagccgcgatcctctcccgccgcgcaggcctgaaggcagacgtccgcggcggccgaaccgcctggtacttcgtgccgaacgccttcctccgcgagttcgccgccctcctggtccagtaccgcaccctcaacgccctggcagcctgcaccgcactctccgtcttcccgctcctggcctacggcgtctgaaaagcacgacccggccgaccggacaccgcgtcacagatccagcggcgccgacccgaaggccacgttgaaccggtcgcaccacaccacgacgctgcgcagccccgacaggtccacgtcctccggaatcaggtagttctggttgccgtcggtggccttcatgggaccgagcggcaggtagcgcccatcgtcgtacttgccccactccccacccgcggtcgcgtcggagagccagatgtgcaggtcgggcccgtccgaggtggagaacccatccagccgcagcacgcgcgcggccccgctgcgcagcacggtggcggtgccccgcgtctcgtgctcctgggtgacgaacccacccgttgccagcaccgtcggctggtcggcggtcgccgacgacgtcccaccgggcgtcgtggcaccgctgcccgccgccgccccggtgctcgacgccccagccccggtgctcacccccgcaccggtcgagacgctgaactcggcgggcagcgcctcgtccgcctcgctgcgcgtccacaaccgccacggctggaacacccacagcccgacgaccgcagccaccaccacaacccccgacaccgcccaaaccgctctcctgcgcaccgaaccgcgcaccacgtcccctcccgttctccgcagacgacctgccaccatgccacgggtcgcgcccgatgaccacgaccaccgcgccacacccgccccacgcagcgactaggctgcccaccggggtcgccagccgatcccgagcgggttgagcaggcagcccaccgcagttcgcgctagtgggatggagggagcgggccggtgtccgagctggatgcagccgcggtggtcacggtgggttccgacgtggtgcgcggggtgcccgtgctgcgcgtcgccggggagatcgacaccaacgtcgccgacgaggtccgccgggcgctgctgccctggctggacgggttgcgcgggccaggggtgctcgacctgaccggggtgaggttcatggcctccaccgggttgtcgctgctgatcgaggccgcccggcgcaggccggcgaagctggtgctggccaccgcccagcgcggcgtgctccggccgctgcagctgaccgggatgagcgcgctgctcccgacgcaccccaccgtggacctggccgtggacgcccagctcggggccgccctggccgggatgcccagcacggcctgaccaccctcggtccacgggcggcctgcccgcggaccacccgcacggcgccctggggggacgagatcacagctggtggaagacgcgatcctggtccgcgcgccgcgacgccggtgggcgcgggcgctcccgccacggcggcggacccgcccccggtccccaccacggctccggcaccggccccgacaccgacaccgaccccagccccgcccctgggcacgaccacaccaccaaccccggtcctgggcgcaggtgtcgccaccgccaccgccctgacgctggcactcgccggggccgcagccccagccgacaacagcgcgggaaaggcggccatcatggacgaggtggacgccccaaccaccccacccaccccggccgcgctggacctcacgccccgcccacccctggccgaggtgcgccgctggaccggcgcgctgctgatcgacgccgacgaggaagcagcggacgacgtgctgctcgtggtcaacgagctggtcgccaacgcctacgaccacaccacctccccactcgccctgcgcctcaccaccacccccgagcacgtgcgcgtggaggtcgaggacggctcccccgacccaccacgcccggacctcaccgcgggcctgcgccagatcggcacgcgcggacgcggcctgctgctgatccgccagctgaccgatcgctggggcagcacgccccaccccggcggcaagaccgtgtgggcggagctgccgaacgtcccggcgacctgagcccgacgccccaccaacgaggccacggcggatctcacgggaagagcgcggcggggcactccgggcgcgttggacgccggcgcactccccggtgaggggtcgggcggcggagtggatgagcgtggcggcgagcagggccggtccggcggacgagacggccatcagcagcccgccgaccggggccgcgaggcgtcgggcgcgggcgccgagcctgcccggcaccgggccgaccacggagctgacgccgaggacggcggtgcaggcgccgagcgcgcgcctgcgggccgcgccctcgaagcgcacctggacggcggtcagcgccccggagaccatgagcgccgcgcccgcgccctggaccacccgcgcggccaccagcgccgggccggtgggggtgaggccgcaggccggggaggcggcggtgaaggtggcgggcccgacgaggtgggcccagcggcggccccggatctcgccgaggtgggccccggtgatcagcaggacggcgagctgcggcaggacaccgacacgggcacgaccgcgtcgatgtgcgtcgcggcggcgcagggcgcggagacgggcgcgggcgagcgctgagggcccgcccggcgccactcccccgtcacacctcccgccgcagcacatcctcctccgtctcccgccgcaccagcacccgcgccactccgtcgcgcacgcccaccacaggcggcctgcccacggcgttgtagttcgacgccagcgcgtggtggtaggcgcccgtcaccggcaccgccagcaggtcccccgcgcgcacgtccgcgggcagcggcacgtcctcggcgagcacgtcacccgcctcgcagtgcctgcccaccaccgtcaccggcgcgcgccgcccgccccggccgaccaggcgcaccgcgtaccggctcccgtacagcgcgggcctggggttgtcgctcatgcccccgtccacggccacgaacacccgcctcaccccgcgcttgacggcagccacccggtacagcgtcacaccagcgcccgcgacgaccgaccgccccggctcgatcagcagcctcggcaccggcacgcgccgcagcgcgcactcgtggctcagcgccacccgcacccggtgcgcgaacccgccaaggtcgaactccccctcccccggcaggtagggcaccgcgaacccgccgccgaggtccagctgctcgatccgcaccccgcacgaggcgatcagcccgaccatccgccgcgccgcctcctcgtacaccgcgacgtgtcgcacctgcgacccgacgtggcagtgcagccccaccagcctcagcgacggctgctcgaccacccgcagcaccgcctccagcgcgtccccacccgccagggagaagccgaacttctggtcctccaccccggtcgccaccgcccggtgggtgcgcgggtcgacgccgggggtgacccggaccagcacgtcctgcggccccctggccagcgcgcccagctgctcgatctcgtcgaacgagtccaccaccacccgcccgaccccgtacccgagggcggccttgaggtcctcgggcgtcttgacgttgccgtgcagcagaatccgctccgccgggaacccgaccgaccgcgcgatcgccagctcccccgccgagcacacgtccagcgacagcccctcgtccgccacccaccggtacacctcgcggcacggcagcgccttgcccgcgaacaccacctcagcctccggcagcacctcccggaacccgcgcgcccgcgcccggaccgtgccctcgtcgagcacctggcagggcgtgccgaaccgggcggcgagctcggtcgcgggcaccccgccgagcagcagctccccccgctccagccgggtccccaggggccacagccccgcctccagggccggttcgccggtcatgccgacgctgggcagcaactccgcgagtgtcatgcccgccagcacacgcccgaaccggccggggcgacagcggcgcgaacgcgtccctgacggcgtgccgggcgggattgacgccgccctgacccgaccgccccagcccgctctcgaacccggcggaagcacccccgaaacgcgccggaaacccgcccgcgcattcccccgaacgcctacctcacggcgattttgatgctttttttacgccgggacgccgcgatattcactcctccgagccgcgcggggacgttgacttctcatgcccgacgacgtgatcgaggagagaccccgaatgtccgaaacaccggttttcgccgttccacccagggtggaaagcccggtacgcccggccgcgcccgccaaccgggtggggcgctggctgctggagcaccgggtgcaaccggcgggacccgcgggcaccgaccagcacagcacgccccaggcgtggtggaaggtcatgtgcctgaccggcgtcgactacttctcgaccctgtcctacctgccgggcatcgcggcgctggcggccggggcggtctcgccgctggcgacgctgctgatcgtcgcgctgaccctgttcgggatgctgccgatgtaccgccgggtggcgcacgagtcgccgcacgggcagggctcggtggcgatgctggaggacctgctgccgttctggcgcggcaagctgttcgtgctggtgctgctgggtttcgtggccacctcgtggatcatcacgatcaccctgtcggcggccgacgcgtcggtgcacgcgctggagaacccgcacgcgcccgcgttcctgcacgggcacgaggtgctggtcaccgtggtgctgctgctcgtgctgggcggggtgttcctgctgggcttcaccgaggcggtcagcgtggccatcccgctggtcgcggtgttcctgctgctcaacgcggtggtcgtggtcgccggcgtgctggaggtgatcgcgaacccggacgtgctggacggctggttcgcggcgctgacctccaccggcggcggcggggtgctgggcgtggtcggcccggccctgctggcgttcccgctgctcgtgctcggcctgtccgggttcgagaccggggtgagcatgatgccgctggtcgaggcgaagggcgccgacgacgccgaacgcctggcgaaccgcgtccgcaacacccgcaagctgctcaccaccgccgcgctgatcatgtcggtgtacctggtggccaccagcttcgtgaccaccctgctcgtgccggtcgagcagttccgccccggcggcgaggccaacgggcgggcgctggcctacctggcgcacgagctgctcggcgagtgggtcggcacggcctacgacatcagcagcgtgctgatcctgtggttcgccggcgcgtccgcgatggccgggctgatcaacatcgtgccgcgctacctgcccgcgtacggcatggccccggactggacgcgcgccgtccgaccggtcgtgctggtctacacggtgatctgcgtcggcatcacggtgatcttccaggccgacgtggacgcccaggccggcgcgtacgcgaccggcatcctggcgatgatggtgtcggcgtcggtggcggtgaccctgtcggtggcgcgcgccgggcggcggggcgcggcctcggcgttcgcggtgctgaccctgatcctggtgtacgcgctggtggagaacgtgatcgagaagccggacggcatcacgatctcgttcgtgttcatcgtcggcatcatcgccgtctcgctggtctcgcggatctcgcgcaccaccgagctgcgcgtggagcacatcgagttcgacgagaccgcgcgcaggctcatcaccgactcgatcgcccacgacggcgcgctgaccgtgatcgcgaaccgcaggcaggccggtgacgtggccgagtacgcggacaaggaggccgagcagcgcggggtgaacccggtgccggggcaggcggacgtgctgttcctggagatcgacgtggtggacccgtcggacttcagcgacgtgctggaggtgcgcggcgtggaggtgggcggccaccgggtgctgcgcgcggacagcccggcggcgccgaacgcgatcgccgcgatactgctggcgctgcgcgactgcaccggggtgcgcccgcactgccacttcgcgtggagcgagggcagcccgctggggcacctgttccgctacctgctggtggggcgcggcgacacggcgccggtggtgcgggagatcatccgggcgcacgagtccgacccggagcgcaggccgggcatccacgtgggggcctgagcgggcacgacggcggggtggtccaggcaggcagcgtggtccaggccagtggggtgctcccggccagcaacgtgctcccggccggtgggggctccagggcgctgcggcggccgatcgcgcgggcgtggtcggcgaaccgctcgcagtgctcgctgagcagggccgcgtcgacggcggcgtcctcaacgccgcgcagcacggccagcacggaccggggcactcaccaaacgcgaagagccacaccaactgggcttcggcgtgggaggcgcggtgcagcggtttgtggtctcgcgctgccgcgcggcgcgggggactgggtcgcgagcagcacctggccgccgtgccgcgcggcgccccgcgccaggtcgcacacggcggccaggtccggcaccggcgcgtcccgccggtcgtggaacacgtcgcgcatcgcgctctccctcggaggatcggatcggaaggccctgatcccaaccgggcgcgcaccccggcgacaagccctcacccgccgaacttgcgctttccttccgccccgacccccgcccgtcacaaacccccgtcaccccgccgtcactttttgtgatgacgatcaggaaacagtagtagcccattcgtgacctgcactgacgcgcagatcaccccacccgtcaacgaaacgtaaaaccgcctggtcaccccgtcaaagacccgtcagcaccccgctcacggcgttttccccgttgcacccttttggcgtcgcggtccccacgaacgggggccgctcggagtcgggaagggagcacgctcatggccgacctggcctacgcgtcgctgctcatcgctgtgttcggactgctcgtcctcggcattcgcggactggggcggctctgatgggcggcacgggagtcgtggccaacgccgtcggtggcgtgctggccctgctgctcatcgggtacctgttcgtcgcgctgatcaggccggagaagttctgatgtcctcgaccacggcgggcctgctccaggtcgccctgctcatcgccgcgctggccgccgcctaccggccgttcggcgactacatggcccgcgtctacaccgacgccaagcacaccaaggtcgagcgcctgctctaccgcgcagcccgcgtcgaccccgactcgcagcagcgctggggcacctacgcgcagggcgtgctcggcttctccctcgtcggcgtggccctgctgtacctgatgcagcgagtgcagccctggctgccgttcgaccacgaccggggcgcggtctcgcccggcatggcgttcaacaccgccgcctcgttcgtggccaacacgaactggcagtcctacgtcccggagaccgtcctcggccacaccgtgcagatggccgggctgaccgtgcagaacttcgtctccggcgcggtcggcatggccgtcgccgtggcgctggtgcgcggcttcacccgcgagggctccgaccggctcggcaacttctgggtcgacctcaccaggggcaccctgcgcgtcctgctgcccgtgtcgttcgtgttcgccatcgtgctggtcgcgaccggcgtcgtgatgagtctgaaggcgggcgtggacgtggacggccagcaggtcgccatcgccccggccgcctcgcaggaggccatcaaggagctcggcaccaacggcggcggcatcttcaacgccaactccgcccacccgttcgagaaccccaacggctggtcgaacctggtcgagatcttcctgatcctgctgatcccggtctcgctcacccgcaccttcggcaccctggtcggcaaccgcaagcagggctacgtgctgctcagcgtcatgggcgtgctgtggaccgcgatgctcgcggtcatctgggcggccgaggcgcacggcctgcgccccctggagggcaaggagctgcggttcggcgtccccggcagcgccctgttcgccaacaccaccaccgccacctccaccggcgcggtcaacgccatgcacgacagcctcaccggcctgggcggcggcgcgacgctgctgaacatgctgttcggcgagatgacgccgggcggcgtcggcaccggcctgtacagcatcctggtgatggcgatcatcgcgatgttcctggccggtctgatggtcgggcgcaccccggagtacctgggcaagaagctgggccgccgcgaggtgacctgcgccgcgctgtccatcctggcgatgcccgcgctggtgctggtcggcgccgggatctcggcggtgctgccgtcgacggccgggtacctgaacaaccccggcgagcacggcctgtccgagatcctctacgcctacgcgtcggcctcgaacaacaacggcagcgcgttcgcgggcatcaccgtgaccagcgactggttccagtcctcgctcggcgtctgcatgttgctcggccggttcgtcccgatcatcgcggtgctgtgcctggccggttcgctcgcccggcagaagcgcgccccgcggaccgcgggcacgctgcccacggacagcccgctgttcgcctcgctgctggtcggcgcgatcgtgctcgtcgccgccctcaccttcgtccccgccctcgccctcggccccatcgcggaggcactgctgtgaccaccaccgacacccgccagcccgcccccgaggacacgggcgcgcggcccccggccaagcccgtcccgtcgggcgtgttcgccccgcgccagctgctcacgtccctgccggacgcgctgcgcaagctccacccccgccaccagctgcgcaaccccgtgatgttcgtggtgtgggcgggctcggtcctggtcacggtcttcgccgtcaccgacccgaacccgttcacgatcgcggtcgcgctgtggctgtggttcaccgccctgttcgccaacctcgccgaggccgtcgccgaggggcgcggcaaggcgcaggccgagtcgctgcgcaggactaagaccgacgcgctggcccgcctgaccgacggccgcaccgtgcccggcaccgagctgaaggtcggcgacctggtcgtggtcgaggccggtgaggtgatccccggcgacggcgacgtggtcgagggcatcgccaccgtcgacgagtcggcgatcaccggcgagtccgcgcccgtggtgcgcgagtccggcggcgaccggtgcgcggtcaccggcggcaccaccgtgctgtcggaccggatcgtcgtgcgcgtcaccagcaagccgggcgagacgttcgtggaccggatgatcgcgctggtcgagggcgcgcagcggcagaagacgccgaacgagatcgcgctgacgatcctgctgtccacgctcacgatcatcttcctgctcgcggtgctcgcgctccagccgttcgcggtgtactccggcggcgagcagtcggtgatcgtgctgaccgcgctgctggtgtgcctgatccccaccacgatcggcgcgctgctgtccgcgatcggcatcgcgggcatggaccgcctggtgcagcgcaacgtgctggccacctcgggccgcgccgtcgaggcggccggtgacgtggacacgctgctgctggacaagaccggcaccatcacctggggcaaccgccgcgccaccgagctgatccccgcgcccggcgtcacgctggacgagctggtggacgccgcccggttgtcgtcgctggccgacggcacccccgagggccgcagcgtggtcgagctgtgcgcgaccgggcacggccgctcccccgagcccaccgacgcggagaagaccggcgagttcgtgccgttcaccgcccagacccggatgagcggcatcgacctggacggccgcagcgtccgcaagggcgccgcgaccgcgttcaccctcaccgactcggtcaagtccacggtggacgagatcagcggcgacggcggcaccccgctggtggtcgccgacggcgagcgggtgctcggcgtgatccggctgtccgacgtggtcaagcccggcatgaaggagcggttcgccgagctgcgcgccatgggcatccgcacggtcatggtcaccggcgacaacccgctgaccgccagggcgatcgcggccgaggcgggggtcgacgactacctcgccgaggccaagcccgaggacaagatggccctgatccgcaaggagcaggagggcggcaagctggtcgcgatgaccggcgacggcaccaacgacgcgccggcgctggcccagtccgacgtgggcgtggccatgaacaccggcacctcggccgccaaggaggccgggaacatggtggacctggactccgaccccaccaagctcatcgagatcgtggagatcggcaagcagctgctgatcacgcggggcgcgctgacgacgttctcggtcgccaacgacctggcgaagtacttcgcgatcctgcccgccatgttcgccgcgatccacccgcagctggacaagctcaacgtcatgggcctggccacgccgcagtcggcgatcctgtcggcggtcatcttcaacgcgctgatcatcgtggtgctgatcccgctggcgctgcgcggcgtgcgctacaagccctccagcgcgagctcgctgctgcggcgcaacctgctggtgtacggcgtcggcggcatcatcacgccgttcgtcggcatctggctcatcgacctgctcgtccgcctcatccccggaatcgggtgaactccgtgaacgcgttcgtgaagcaggccctggccggtctgcgcgtcctgctggtgctgaccgtcatcaccggcgtgctctaccccgccgccgtctggctcgtctcgcgggtgcccggcctgcacgccaacgccgaggccaccggcaccgagctggtcgtggcgccgcgcgagggcgacggctggttccagccgcgcccgtcgatggcgacgctgcccgcgtcgggcgggtccaacaagggcgagcgcaacgccgactacgacgcggtgatcgccgagcgccgcaccgagatcgcccggcgcgagggcgttgcggaggacgccgtgccgcaggacgcggtgaccgcctcggcctccgggctggacccgctgatcagcgccgagtacgcggcgatccaggtgccgcgcgtggcgcgggagcgcggggtgtcggaggacgccgtgcgggcgctggtcgccgaggcgtcggtgggccgctcgctcgggttcgtgggcgagccgggcgtcaacgtcaccgccctcaaccgggccgtcgacgcggcggagtgagaccgaccgggggccgtcctcgcggcggcccccggtcttccccatttctctgatctcgggagcgggcgggaccgtggacaagcgcaagcgcggcgaactgcgcatctacctgggcgcggcgccgggcgtcggcaagaccttcgcgatgctcggcgaggcgcaccgccgccgggggcgcggcgcggacgtcgtcgtcgccctggtcgagacgcacggccgcgagcgcaccgccaccatggtcgacggcctggaggtgctgccccgcaaggaggtccagcaccgggggaccacgatcaccgagatggacgtggacgcggtgctggcccgcgcgcccgagatcgccgtggtggacgagctggcgcacaccaacgcccccggctcccgcaacgccaagcgctggcaggacgtcgaggagctgctggacgccggcatcgacgtgctgtccacgctcaacatccagcacctggagtcgctcaacgacgtggtgcgccgcatcacccgcgtcgagcagcgcgagaccatccccgacgaggtggtgcgccgcgccgagcaggtggagctggtcgacctgaccccggaggcgctgcgccgccgcctggcgcacggcaacgtctacgccgcgcacaagatcgacgccgcgctgggcaactacttccgggtcgggaacctgaccgcgctgcgcgagctggcgctgctgtgggtggccgaccaggtggacgtggcgctccagcggtaccgcaccgagcagcgcatcaccgacacctgggaggcccgcgagcgggtcgtggtcgcggtgaccggcggcgcggagagcgagaccctgatccgcagggcccgccgcatcgccgcgcgcgccggggcggagctgctggtggtgcacaccatgcgcggcgacggcctcgcgggttccgcgccggagtcgatccggacccgcgtcgggctcaggtgctcgacggtgctcttcaacgtggtctcctcgtaacgggacgtgcggaacaccccgcagcgcccagggtcgggcggctgacgggattcgcctgagtctaggcgaggccgcccccggccggggtggcaccccgcgaccgggtggttcacgtgcgggtgcgcgcgcccggcgcggcgcgcgcggtgcgagaggtgggccgtccggcggcgcgcggttttccgacatggcgcgcgcacgaaatagttttcggcgggtcgggcgccgtcgaatcgactcggggtcgggttttccgcgccaccccggaagcggacgaaccgggcgggcgaaccgggcgggcggtgcgcggacaacgggcgcgaccgccgcggtgcgccggtttgggcagcctttaccgccctccaggtcacccattccgccgttgcggggaacatccgcgtaccagtggcccccggcggacacgcggcccagcacccgctaggccgttcgcaggacgtcgtggtgcaccgggagcgtgaaaccgaacgtaaccggacagcggcgggctcaagtggggtaacactggcgccgcagcgcactcttacccacagcgacgaacgcggcggaacgctaccctttacaggtgaagtgaggccattcggagcaccggtgcgcagaaaactttcacgcccggagatgactccactcgccgtagtccattagtgtgggattccggtaccgttgcgccgcaggccgcaagaaggcggccaggaaagacgattaactcatccgggcgccccgccgtcgtgcacgtgaacgcgacgggcgaccgggaacggaacgagcgagacatgtcatcgcgctctttaccacctaccagaaaaggtgccgatgaccccgatgaagaccattccgccgattcccccgaacacgcgggcgtccgcccgtccgccgctcgggcaaccgcacgacgggcttgcgcgccacccggaaccccagggcccggccgcgaggcgatgttgacccgacccccggccaccagccgggacaggccgaccaccgccacgcgcggaacccacggcgaaccgcctctcgccgtgatccaccacggaccgttggggagttccatggagacccgtcaacttctggcgttcaccacagtggtgcagaccggcagcttcacgaaggccgccgccacgctgaactgctctcagcccacgatcaccaccaggatcaaggcgctggaggagaccctcggcgtcgccctgttccgcaggttgccgcgcggcatccagatgacctccgccggggtcgagctgctgccgttcgcgcgcaacatcatcacgctcaccgacaaggcccgcaaggcgatcaccatgaacggggagccgcacgggcacctcgtgataggcagcgcccagagcctcaccgactaccggctcttacccctgatcgagtacatgtgctggcgctacccgagcgtccagatctcgctgcactcgcgaacaacccggtcgaacctggccgccgtgcgcgagggcaggttggactgcgcgttcttcatcggcccggtcgagcagcgggacggtctggagacgacggtgctgtgccccgaaccgctggtgatggtcgcgggccccggccacgcgctggcgcggtcgggcgcggtcaccgaggcggacctgcggggcagcacgctggtcagggccgagaacggggcgagctaccacgagcagttcgagcgggcgctcgggctgcacgaggccgagtcgcgatcgccggtgctggccctggactcggtcgacgcggccaagcgggcggtcgcctcggggctgggcatctcgctggtgccggaggtcacggtcgccgcggagctggcggacggcaggctcagccgcatcggctggaccccgccgttccgggtgttcacccagttcgcgtggcgccaggacaactcggcgaacccgtcggtgaccgcgctggtctcggcggcggcgcaggtggtgagcgagcaggtggccgcgacacccgcgtagggcgtcgacgtgcagggtcgtggatgcggagcggccccctcgtgctgcgcagagggggccgagaccgtcggggcgacaggatttgaacctgcgaccccccgctcccaaagcgggtgcgctaccaaactgcgccacgccccggtcaccaggagcttagcgcgacgcgctaagctgttttcagcacccacccggtgggcgctgcgcgggtgtagctcaatggtagagccccagccttccaagctggtcatgcgggttcgattcccgtcacccgctccaccagatcc

SEQ ID NO: 12 - DNA, 27 bp, Artificial Sequence
  Primer
ccscgggcgnycngsttcgacngygag

SEQ ID NO: 13 - DNA, 28 bp, Artificial Sequence
  Primer
cgtcncggannccggagcacatgccctg

SEQ ID NO: 14 - DNA, 47 bp, Artificial Sequence
  Primer
atatactagtcacgtcaccggcgcggtgtccgcggacttcgtcaacg

SEQ ID NO: 15 - DNA, 42 bp, Artificial Sequence
  Primer
atatcctaggctggtggcggacctgcgcgcgcggttggggtg

SEQ ID NO: 16 - DNA, 1421 bp, Actinosynnema pretiosum
atatactagtcacgtcaccggcgcggtgtccgcggacttcgtcaacgaccacgcgttccgggccgcctggcgcggcgacggggcgccggtggacccggtggtcggcgacgtggaggacaccgccagggggttcctcgtcacccggtcggggatctcgatcggggtgcgcgcggcctgggcctcgcaccaggcgctggacaccacggtcgtgcgcgtggagggcagcggcggcacggcggagctgcgctgcaccttcggcttcagcccgaaccgcgagggcccgtcgcggctcctgctcaccaccgacggccgcaccaccccggtcgagctgcccgccgagccggtgggcgccgagtacgacgcccagctcgcgagcctgcccacccgcctggccgaccccgccacgcgcggcgaggcggcgtccggggcgcgctggatcgcgggcgcgatcgaacgggtctaccgcgccgccgacaccgtgcggtgcgccgaacgacgacacgcgccggtcgacctcgtcccgacgggagccccgtgatgaccaccggacagaccaacccccgagtcgtgcgggcccgagccggggacgagcgggccgcgaccgcggttccggcgcccgcggccccgcgccccctcgccccgccgacccccgcgccccaccggcacgtccccggcgcgacctacgaccgggccgtgctgttcgacctcgacggggtgctggtcaacagcttcgccgtcatgcggcaggcgttcgagatcgcctacgccgaggtcgtcggcgacgggcccgcgccgttcgaggagtacaaccggcacctggggcggtacttcccggacatcatgcggatcatggacctgccgctggagatggagggtccgttcgtccgcgagagctaccggctggcgggtgaggtggaggtgttcgagggcgcgccggagctgctggcggacctgcggcagcacggcttcggcaccgccgtggtcaccggcaagagcgggccgcgcgcccggtcgctgctgaccaccctcggcatggcggggctgttcgaccatatcatcggctccgacgaggtcgcgaaccccaagcccgcgccggacatgctcctgctggccaccggcctgctcgacgtcccggccgaccgggtggtgatggtgggcgacgccctgacggacctggccagcgcccgcgccgcgggctacccggcgctggccgcgctgtggggcgagacggacgaggcggagctgctggcggcgaacccggacgcggtggtccgcaagccctcgcaggtgctcgactggtgcctggcccacctcgccgacgaccggacgtgacgcgcgagcaggccccggcgcggcacgtgccggggcctgcggggcgtcaccccaaccgcgcgcgcaggtccgccaccagcctaggatat

SEQ ID NO: 17 - DNA, 42 bp, Artificial Sequence
  Primer
atatcctaggcaccacgtcgtgctcgacctcgcccgccacgc

SEQ ID NO: 18 - DNA, 42 bp, Artificial Sequence
  Primer
atattctagacgctgttcgacgcgggcgcggtcaccacgggc

SEQ ID NO: 19 - DNA, 1423 bp, Actinosynnema pretiosum
atatcctaggcaccacgtcgtgctcgacctcgcccgccacgcgctcaccccccgatcatgatcgactcggtgacgcgggcgaactcgtccttcttcacgagcacccgcgtcgtctccaccccgtccagcacggtgaagtagcggcggccgatcagcacgtgctgcccgttctccgccgcccgaccccggaacgtggtgcccgccagcaccgagtcccgcgcgtagtcgtccagcccgtccaaccgcaccgcctcccagccgtcccggtccgcccgccccggccggaaccggtacaccgcctgccacgccccgtccctgggcctgcgctccaccacgtgcgcgtccccgcgctccacgacccggtaggcgcagccgtactgctcctgctcgaccgccgacagctccagcggctccaggtaggacgggccgacgaaccccacgtccaccagccaggtccgcccgtccaggcgcaccaggttgaacgagtgctcctcgtccggcccgaaccggtcgtcggccagccggatcgccgccgcgaccatcagcacctcgtagcccagcgcggtgagcagggcgtggaagagccggttgagctcgtagcagaccccgccgttgcggccggtcaccacgtgcgcgaacacacggggcagcgggatctccgcgagcccccggttcggcggcaaccggtcggccgcgccgccgttgtcgtagggcacggacatcaggtgccgcttgtgcagcgcccgcaacgactccagcgtcgggctcggcacgccgccctccacgccgatgcgccgcaggtactcggccacgtcgatcatgctggtgctcacctctcgtcggtcgggtcggtggactgccctgcgcctgatcgccctgcgctcgactgccctgcgcctgaatgccctgcgcccggctgccctgcgcccgattgccctgagcccgattgctcggcggccagtcgctcgcgcagcgcctcggcggcgatcgcgggcgtcggctggtcgaacagcagcgcggcgggcaggcgcagcccggtggcggtgttcagccggttccgcagctgcacggccgtcatggagtcgaagccgatctcgaagaacgcgtcgtccgggcccaccgcgcccttctcgccgtgcccgagcacggcggcggccaggtcgcggaccagctcggtgagcacggcctcctgctcgtcgcccgacagcccggccaggcgcgcccgcaacccggccgggggcgcggcctgcccggtggcgggggagagcgagcgccgcgcggcggggacgagcgcgcgcagcagcgcgggcaccgcctcgtcgcgcgcggaggcgcgcagcgccgccaggtccagcggcagcggcacgaccagcgccgggccgcccgtggtgaccgcgcccgcgtcgaacagcgtctagaatat

SEQ ID NO: 20 - DNA, 161 bp, Actinosynnema pretiosum
ctcgccgacgaccggacgtgacgcgcgagcaggccccggcgcggcacgtgccggggcctgcggggcgtcaccccaaccgcgcgcgcaggtccgccaccagcctaggcaccacgtcgtgctcgacctcgcccgccacgcgctcaccccccgatcatgatcga

SEQ ID NO: 21 - DNA, 161 bp, Actinosynnema pretiosum
tcgatcatgatcggggggtgagcgcgtggcgggcgaggtcgagcacgacgtggtgcctaggctggtggcggacctgcgcgcgcggttggggtgacgccccgcaggccccggcacgtgccgcgccggggcctgctcgcgcgtcacgtccggtcgtcggcgag

SEQ ID NO: 22 - protein, 22 aa, Actinosynnema pretiosum
VAGEVEHDVVPRLVADLRARLG

SEQ ID NO: 23 - DNA, 30 bp, Artificial Sequence
  primer
ggtcactggccgaagcgcacggtgtcatgg

SEQ ID NO: 24 - DNA, 36 bp, Artificial Sequence
  primer
cctaggcgactaccccgcactactacaccgagcagg

SEQ ID NO: 25 - DNA, 1595 bp, Actinosynnema pretiosum
cctaggcgactaccccgcactactacaccgagcaggcctacgcctacgggaactcctggacatcaccgaccacaccgtccaacgcaacttccgcgaactggccgatctggtaggcgacgcgaagggcctgctgttccacccacgcgacctggtgggcgtcccagaattcggctgcttcctagcagtagccgaacacccgtaaccacgcggtggcgtcccccacggacgccaccgcctcgcgggctgcggggcgagcgcagcgagcccgcgcagccccactcccgcgtccctcttctccgtgtggcctggcgcatgtcaaattcccactgactgccaacagatcatgtgccgtttgagcaggtcagcgacttgtcgcgcttcggtgccttaaggccgagctgggatgggggcactgtttccggactgagcggggcagcttggaaggtggagttcggtgagcagaggcagcacgtcccgtcgcacgtagaggtggttgtacacgcggtggcgggacctgcgcagtaggccgctatccgcaagctgctccaagatcaggagtgcggcgcggtgcgtatagccgagttcggcggtcagcatggtgctgttgagcagtggggcgacgagcagcggggcgggaagcgctttgaccttcctccgcccggtgcgcatcgcccaggtgggcgatcgcgcgagcctcacggatcgcggtcacctcatgcaggctggcgctcaacctggaacgcgcgactgtttcgtccagacgtgccagggcggtgtaggcgtgcaacaaggtcttgctggtttcggagcgcagtctgagccgggaccaggacgacaactccgcgatcctcgcggacgggggcggcctcgtgtcttcaccggtggtagttgacctgcgcggggcggaggtgccctattgctgccgggacgaggtcatcccccggagcagtttctcagcacgccgtgaatcgagatccggggcgctgagcgcggtgaacgcctcgtccagcgagtcgcacgcgcacgtcgtcctgacatcgggccgcgcatggcccgaggtggtcagcggtgagcgggaaggcgcggcagggtgtgtgcgagacactccgggactccgtgcagaaggtcgatcaggcgaaagggttgaactgcgaatcgcaaagcggcccggccgcaaaggggtcgggccgcctgcgacgattggtcacgctgctgcggcgcggtcccgccggaactgcttgccgagcaggtcgatccgccccttgtgatcttctgccagcgcctccagaaccgagagcagtcgtcgggcgtgcagtgcatggccaataccatcgtcgcgtaccccagagggtgtcgctcccgttcaggggcgaccatttcccacgcccgcttggcctccttggcggcccggccaagatcgccgagcatcaggtaggtgcccgacaacccgacaaccctgcctgccaacgcggcttccggcaccccgcgcgcctcgtcggcttccaacgcccgaacaccgtgccacagcacggcccgcgcgttgccctcgctcgtctccagccatcccatgacaccgtgcgcttcggccagtgacc

SEQ ID NO: 26 - DNA, 30 bp, Artificial Sequence
  primer
cctaggaacgggtaggcgggcaggtcggtg

SEQ ID NO: 27 - DNA, 31 bp, Artificial Sequence
  primer
gtgtgcgggccagctcgcccagcacgcccac

SEQ ID NO: 28 - DNA, 1541 bp, Actinosynnema pretiosum
gtgtgcgggccagctcgcccagcacgcccacgagggtctccagcgcgtccgcgccggtgcgcgcgccccggacgacctcgaccgtggggatcaggtacggcgggttcatgaagtgcgtgccgatcagccgcgccgggtcggggacgtgcccggccagctcgtcgatcgggatcgaggaggtgttggacaccagcggcacgcgcggcccggtgagcgcggcggccccggccagcacctcggccttgaccggcagctcctcggtgaccgcctccaccaccagcgagacgtccgcgacgtcggcgagcgaggtggtggtgagcagctcgccccgctcgcggtcctcgggcagcgcccgcatcagcctggccatgcgcagctgggcggccaccgcctcccgcgcccgcccgaccttggcccggtcggtctcgaccagcaccaccggcacgccgtgcccgacggccagggaggtgatccccaggcccatcgtgcccgcgccgagaacggcgagcaccgtcctgccgtcctgctctcccatcgcgctcccccgccgcggccaccgcggccgccgtccggtccgcgcgccgtcccggcacgcgcattccaccctcgatcgtgtgccgggaaaggcgcgcccgaccccctgacctgcccccctgaacccccctcaacggaaccggaaatcgaatgtcccgaacgcgccgtcaaatcgtcgattgacagccgcagaactgttcatagactgtggcggcagtaccgatctccgaattccacggaagagtcctcccccatggctcagcagatcagcgccacctcggaaatcctcgactacgtccgcgcgacctcgttgcgcgacgacgacgtgctcgccggtctgcgggagcggaccgcggttctcccggccgcgtccgcgctgcaggtggccccggaggaggggcagctgctcggcctgctggtgcgcctggtcggcgcgcgctcggtgctggaggtcggcacctacaccgggtacagcacgctgtgcatggcccgcgccctcccgcccggcggacgtgtcgtgacctgcgacgtcgtcgcgaagtggccggacatgggcaggccgttctgggagcgggcgggcgtcgcggaccgcatcgacgtccgcgtcggcgacgcccgcgccaccctggccggcctgcacgccgagcacgccgtgttcgacctggtgttcatcgacgcgaacaagtcggattacgtccactactacgagcgcgcgctgacgctgctgcgcaccggcggcctggtcgtcgtggacaacacgctctttttcgggcgggtcgccgatccgtccgcgaccgatccggacaccaccgccgtgcgcgagctgaacgcgctgctgcacgccgacgagcgggtcgacatgtgcctgctgccgatcgcggacggaatcacgctcgccgtgaagcggtgaacccgcccgaatcgcgccgaattcccccggagagaaaggccgccgcagtgttcaccgaggacgtggccaccgacctgcccgcctacccgttcctagg

(19) United States
(12) Patent Application Publication
(10) Pub. No.: US 2009/0253667 A1
(43) Pub. Date: Oct. 8, 2009

(54) 18 ,21-Didesoxymacbecin Derivatives for the Treatment of Cancer

(76) Inventors: Christine Martin, Essex (GB); Ming Zhang, Essex (GB); Nigel Coates, Essex (GB); William Vousden, Essex (GB); Steven Moss, Essex (GB); Sabine Gaisser, Essex (GB)

Correspondence Address: DANN, DORFMAN, HERRELL & SKILLMAN, 1601 MARKET STREET, SUITE 2400, PHILADELPHIA, PA, 19103-2307, US

(21) Appl. No.: 12/296,542

(22) PCT Filed: May 9, 2007
(86) PCT No.: PCT/EP07/54476

§ 371 Date: Apr. 17, 2009

(30) Foreign Application Priority Data
May 9, 2006 (GB) 0609132.6
Aug. 22, 2006 (GB) 0616637.5
Nov. 9, 2006 (GB) 0622341.6

(51) Int. Cl. A61K 31/395; C12P 17/10; C12N 1/00; C07D 225/06

(52) U.S. Cl. 514/183; 435/121; 435/243; 540/461

(57) ABSTRACT

The present invention relates to 18,21-didesoxymacbecin analogues that are useful, e.g. in the treatment of cancer, B-cell malignancies, malaria, fungal infection, diseases of the central nervous system and neurodegenerative diseases, diseases dependent on angiogenesis, autoimmune diseases and/or as a prophylactic pre-treatment for cancer. The present invention also provides methods for the production of these compounds and their use in medicine, in particular in the treatment and/or prophylaxis of cancer or B-cell malignancies.

### BACKGROUND OF THE INVENTION

The 90 kDa heat shock protein (Hsp90) is an abundant molecular chaperone involved in the folding and assembly of proteins, many of which are involved in signal transduction pathways (for reviews see Neckers, 2002; Sreedhar et al., 2004a; Wegele et al., 2004 and references therein). So far nearly 50 of these so-called client proteins have been identified and include steroid receptors, non-receptor tyrosine kinases e.g. src family, cyclin-dependent kinases e.g. cdk4 and cdk6, the cystic transmembrane regulator, nitric oxide synthase and others (Donze and Picard, 1999; McLaughlin et al., 2002; Chiosis et al., 2004; Wegele et al., 2004; http://www.picard.ch/downloads/Hsp90interactors.pdf). Furthermore, Hsp90 plays a key role in stress response and protection of the cell against the effects of mutation (Bagatell and Whitesell, 2004; Chiosis et al., 2004). The function of Hsp90 is complicated and it involves the formation of dynamic multi-enzyme complexes (Bohen, 1998; Liu et al, 1999; Young et al., 2001; Takahashi et al., 2003; Sreedhar et al, 2004; Wegele et al., 2004). Hsp90 is a target for inhibitors (Fang et al., 1998; Liu et al., 1999; Blagosklonny, 2002; Neckers, 2003; Takahashi et al., 2003; Beliakoff and Whitesell, 2004; Wegele et al., 2004) resulting in degradation of client proteins, cell cycle dysregulation and apoptosis. More recently, Hsp90 has been identified as an important extracellular mediator for tumour invasion (Eustace et al., 2004). Hsp90 was identified as a new major therapeutic target for cancer therapy which is mirrored in the intense and detailed research about Hsp90 function (Blagosklonny et al., 1996; Neckers, 2002; Workman and Kaye, 2002; Beliakoff and Whitesell, 2004; Harris et al., 2004; Jez et al., 2003; Lee et al., 2004) and the development of high-throughput screening assays (Carreras et al., 2003; Rowlands et al., 2004). Hsp90 inhibitors include compound classes such as ansamycins, macrolides, purines, pyrazoles, coumarin antibiotics and others (for review see Bagatell and Whitesell, 2004; Chiosis et al., 2004 and references therein).

The benzenoid ansamycins are a broad class of chemical structures characterised by an aliphatic ring of varying length joined either side of an aromatic ring structure. Naturally occurring ansamycins include: macbecin and 18,21-dihydromacbecin (also known as macbecin 1 and macbecin II respectively) (1 &2; Tanida et al, 1980), geldanamycin (3; DeBoer et al., 1970; DeBoer and Dietz, 1976; WO 03/106653 and references therein), and the herbimycin family (4; 5, 6, Omura et al, 1979, Iwai et al, 1980 and Shibata et al, 1986a, WO 03/106653 and references therein).

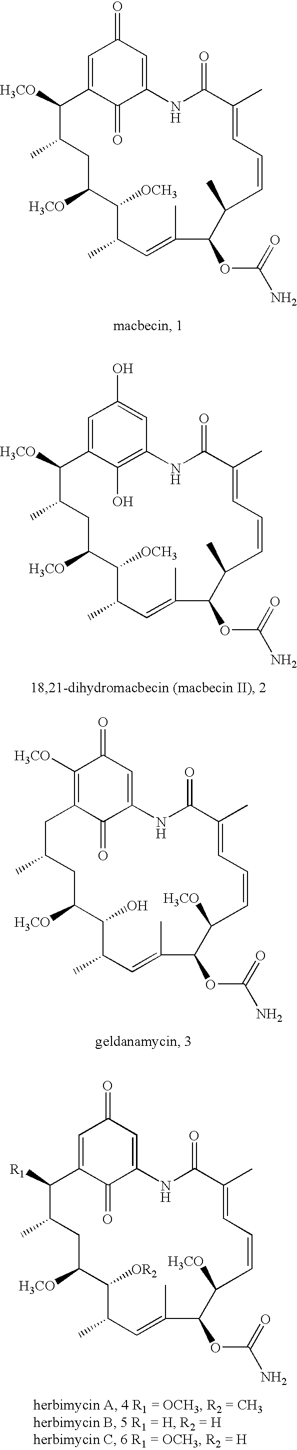

Ansamycins were originally identified for their antibacterial and antiviral activity, however, recently their potential utility as anticancer agents has become of greater interest (Beliakoff and Whitesell, 2004). Many Hsp90 inhibitors are currently being assessed in clinical trials (Csermely and Soti, 2003; Workman, 2003). In particular, geldanamycin has nanomolar potency and apparent specificity for aberrant protein kinase dependent tumour cells (Chiosis et al., 2003; Workman, 2003).

It has been shown that treatment with Hsp90 inhibitors enhances the induction of tumour cell death by radiation and increased cell killing abilities (e.g. breast cancer, chronic myeloid leukaemia and non-small cell lung cancer) by combination of Hsp90 inhibitors with cytotoxic agents has also been demonstrated (Neckers, 2002; Beliakoff and Whitesell, 2004). The potential for anti-angiogenic activity is also of interest: the Hsp90 client protein HIF-1α plays a key role in the progression of solid tumours (Hur et al., 2002; Workman and Kaye, 2002; Kaur et al., 2004).

Hsp90 inhibitors also function as immunosuppressants and are involved in the complement-induced lysis of several types of tumour cells after Hsp90 inhibition (Sreedhar et al., 2004). Treatment with Hsp90 inhibitors can also result in induced superoxide production (Sreedhar et al., 2004a) associated with immune cell-mediated lysis (Sreedhar et al., 2004). The use of Hsp90 inhibitors as potential anti-malaria drugs has also been discussed (Kumar et al., 2003). Furthermore, it has been shown that geldanamycin interferes with the formation of complex glycosylated mammalian prion protein PrP^{c} (Winklhofer et al., 2003).

As described above, ansamycins are of interest as potential anticancer and anti-B-cell malignancy compounds, however the currently available ansamycins exhibit poor pharmacological or pharmaceutical properties, for example they show poor water solubility, poor metabolic stability, poor bioavailability or poor formulation ability (Goetz et al., 2003; Workman 2003; Chiosis 2004). Both herbimycin A and geldanamycin were identified as poor candidates for clinical trials due to their strong hepatotoxicity (review Workman, 2003) and geldanamycin was withdrawn from Phase I clinical trials due to hepatotoxicity (Supko et al., 1995; WO 03/106653).

Geldanamycin was isolated from culture filtrates of Streptomyces hygroscopicus and shows strong activity in vitro against protozoa and weak activity against bacteria and fungi. In 1994 the association of geldanamycin with Hsp90 was shown (Whitesell et al., 1994). The biosynthetic gene cluster for geldanamycin was cloned and sequenced (Allen and Ritchie, 1994; Rascher et al., 2003; WO 03/106653). The DNA sequence is available under the NCBI accession number AY179507. The isolation of genetically engineered geldanamycin producer strains derived from S. hygroscopicus subsp. duamyceticus JCM4427 and the isolation of 4,5-dihydro-7-O-descarbamoyl-7-hydroxygeldanamycin and 4,5-dihydro-7-O-descarbamoyl-7-hydroxy-17-O-demethylgeldanamycin were described recently (Hong et al., 2004). By feeding geldanamycin to the herbimycin producing strain Streptomyces hygroscopicus AM-3672 the compounds 15-hydroxygeldanamycin, the tricyclic geldanamycin analogue KOSN-1633 and methyl-geldanamycinate were isolated (Hu et al., 2004). The two compounds 17-formyl-17-demethoxy-18-O-21-O-dihydrogeldanamycin and 17-hydroxymethyl-17-demethoxygeldanamycin were isolated from S. hygroscopicus K279-78. S. hygroscopicus K279-78 is S. hygroscopicus NRRL 3602 containing cosmid pKOS279-78 which has a 44 kbp insert which contains various genes from the herbimycin producing strain Streptomyces hygroscopicus AM-3672 (Hu et al., 2004). Substitutions of acyltransferase domains have been made in four of the modules of the polyketide synthase of the geldanamycin biosynthetic cluster (Patel et al., 2004). AT substitutions were carried out in modules 1, 4 and 5 leading to the fully processed analogues 14-desmethyl-geldanamycin, 8-desmethyl-geldanamycin and 6-desmethoxy-geldanamycin and the not fully processed 4,5-dihydro-6-desmethoxy-geldanamycin. Substitution of the module 7 AT lead to production of three 2-desmethyl compounds, KOSN1619, KOSN1558 and KOSN1559, one of which (KOSN1559), a 2-demethyl-4,5-dihydro-17-demethoxy-21-deoxy derivative of geldanamycin, binds to Hsp90 with a 4-fold greater binding affinity than geldanamycin and an 8-fold greater binding affinity than 17-AAG. However this is not reflected in an improvement in the IC₅₀ measurement using SKBr3. Another analogue, a novel nonbenzoquinoid geldanamycin, designated KOS-1806 has a monophenolic structure (Rascher et al., 2005). No activity data was given for KOS-1806.

In 1979 the ansamycin antibiotic herbimycin A was isolated from the fermentation broth of Streptomyces hygroscopicus strain No. AM-3672 and named according to its potent herbicidal activity. The antitumour activity was established by using cells of a rat kidney line infected with a temperature sensitive mutant of Rous sarcoma virus (RSV) for screening for drugs that reverted the transformed morphology of the these cells (for review see Uehara, 2003). Herbimycin A was postulated as acting primarily through the binding to Hsp90 chaperone proteins but the direct binding to the conserved cysteine residues and subsequent inactivation of kinases was also discussed (Uehara, 2003).

Chemical derivatives have been isolated and compounds with altered substituents at C19 of the benzoquinone nucleus and halogenated compounds in the ansa chain showed less toxicity and higher antitumour activities than herbimycin A (Omura et al., 1984; Shibata et al., 1986b). The sequence of the herbimycin biosynthetic gene cluster was identified in WO 03/106653 and in a recent paper (Rascher et al., 2005).

The ansamycin compounds macbecin (1) and 18,21-dihydromacbecin (2) (C-14919E-1 and C-14919E-1), identified by their antifungal and antiprotozoal activity, were isolated from the culture supernatants of Nocardia sp No. C-14919 (Actinosynnema pretiosum subsp pretiosum ATCC 31280) (Tanida et al., 1980; Muroi et al., 1980; Muroi et al., 1981; U.S. Pat. No. 4,315,989 and U.S. Pat. No. 4,187,292). 18,21-Dihydromacbecin is characterized by containing the dihydroquinone form of the nucleus. Both macbecin and 18,21-dihydromacbecin were shown to possess similar antibacterial and antitumour activities against cancer cell lines such as the murine leukaemia P388 cell line (Ono et al., 1982). Reverse transcriptase and terminal deoxynucleotidyl transferase activities were not inhibited by macbecin (Ono et al., 1982). The Hsp90 inhibitory function of macbecin has been reported in the literature (Bohen, 1998; Liu et al, 1999). The conversion of macbecin and 18,21-dihydromacbecin after adding to a microbial culture broth into a compound with a hydroxy group instead of a methoxy group at a certain position or positions is described in U.S. Pat. Nos. 4,421,687 and 4,512,975.

During a screen of a large variety of soil microorganisms, the compounds TAN-420A to E were identified from producer strains belonging to the genus Streptomyces (7-11, EP 0 110 710).

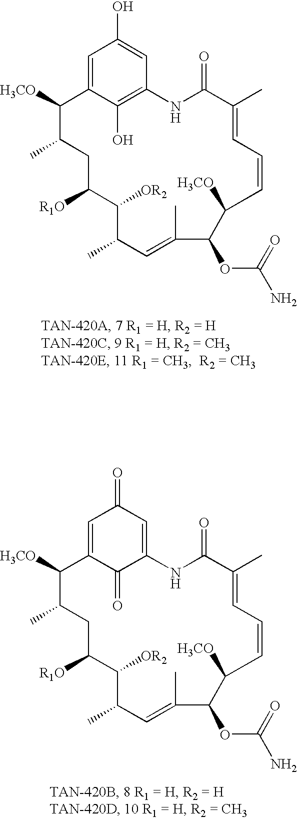

In 2000, the isolation of the geldanamycin related, non-benzoquinone ansamycin metabolite reblastatin from cell cultures of Streptomyces sp. S6699 and its potential therapeutic value in the treatment of rheumatoid arthritis was described (Stead et al., 2000).

A further Hsp90 inhibitor, distinct from the chemically unrelated benzoquinone ansamycins is Radicicol (monorden) which was originally discovered for its antifungal activity from the fungus Monosporium bonorden (for review see Uehara, 2003) and the structure was found to be identical to the 14-membered macrolide isolated from Nectria radicicola. In addition to its antifungal, antibacterial, anti-protozoan and cytotoxic activity it was subsequently identified as an inhibitor of Hsp90 chaperone proteins (for review see Uehara, 2003; Schulte et al., 1999). The anti-angiogenic activity of radicicol (Hur et al., 2002) and semi-synthetic derivates thereof (Kurebayashi et al., 2001) has also been described.

Recent interest has focussed on 17-amino derivatives of geldanamycin as a new generation of ansamycin anticancer compounds (Bagatell and Whitesell, 2004), for example 17-(allylamino)-17-desmethoxy geldanamycin (17-AAG, 12) (Hostein et al., 2001; Neckers, 2002; Nimmanapalli et al., 2003; Vasilevskaya et al., 2003; Smith-Jones et al., 2004) and 17-desmethoxy-17-N,N-dimethylaminoethylamino-geldanamycin (17-DMAG, 13) (Egorin et al., 2002; Jez et al., 2003). More recently geldanamycin was derivatised on the 17-position to create 17-geldanamycin amides, carbamates, ureas and 17-arylgeldanamycin (Le Brazidec et al., 2003). A library of over sixty 17-alkylamino-17-demethoxygeldanamycin analogues has been reported and tested for their affinity for Hsp90 and water solubility (Tian et al., 2004). A further approach to reduce the toxicity of geldanamycin is the selective targeting and delivering of an active geldanamycin compound into malignant cells by conjugation to a tumour-targeting monoclonal antibody (Mandler et al., 2000).

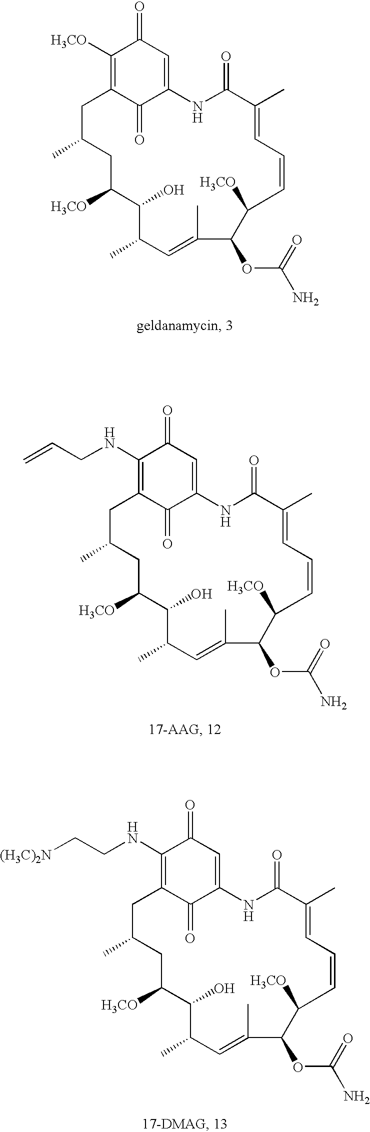

Whilst many of these derivatives exhibit reduced hepatotoxicity they still have only limited water solubility. For example 17-AAG requires the use of a solubilising carrier (e.g. Cremophore®, DMSO-egg lecithin), which itself may result in side-effects in some patients (Hu et al., 2004).

Most of the ansamycin class of Hsp90 inhibitors bear the common structural moiety: the benzoquinone which is a Michael acceptor that can readily form covalent bonds with nucleophiles such as proteins, glutathione, etc. The benzoquinone moiety also undergoes redox equilibrium with dihydroquinone, during which oxygen radicals are formed, which give rise to further unspecific toxicity (Dikalov et al., 2002). For example treatment with geldanamycin can result in induced superoxide production (Sreedhar et al., 2004a).

Therefore, there remains a need to identify novel ansamycin derivatives, which may have utility in the treatment of cancer and/or B-cell malignancies, preferably such ansamycins have improved water solubility, an improved pharmacological profile and/or reduced side-effect profile for administration. The present invention discloses novel ansamycin analogues generated by biotransformation and optionally genetic engineering of the parent producer strain. In particular the present invention discloses novel 18,21-didesoxymacbecin analogues, which generally have improved pharmaceutical properties compared with the presently available ansamycins; in particular they are expected show improvements in respect of one or more of the following properties: activity against different cancer sub-types, toxicity, water solubility, metabolic stability, bioavailability and formulation ability. Preferably the 18,21-didesoxymacbecin analogues show improved bioavailability.

### SUMMARY OF THE INVENTION

In the present invention non-natural starter units have been fed to a macbecin producing strain, optionally in combination with targeted inactivation or deletion of the genes responsible for the post-PKS modifications of macbecin in order to produce novel macbecin analogues formed by incorporation of a non-natural starter unit. Specifically, we describe novel macbecin analogues formed by incorporation of a starter unit which results in a benzene moiety which is either not substituted in the 17, 18 and 21 positions or which is substituted in some or all of these positions by fluorine. Optionally the genes or regulators responsible for starter unit biosynthesis may be manipulated by targeted inactivation or deletion or modified by other means such as exposing cells to UV radiation and selection of the phenotype indicating that starter unit biosynthesis has been disrupted. The optional targeting of the post-PKS genes may occur via a variety of mechanisms, e.g. by integration, targeted deletion of a region of the macbecin cluster including all or some of the post-PKS genes optionally followed by insertion of gene(s) or other methods of rendering the post-PKS genes or their encoded enzymes non-functional e.g. chemical inhibition, site-directed mutagenesis or mutagenesis of the cell for example by the use of UV radiation. As a result, the present invention provides 18,21-didesoxymacbecin analogues, methods for the preparation of these compounds, and methods for the use of these compounds in medicine or as intermediates in the production of further compounds.

Therefore, in a first aspect the present invention provides analogues of macbecin which are lacking the usual starter unit, and which have instead incorporated a starter unit which results in 18,21-didesoxymacbecin analogues which are either not substituted in the 17, 18 and 21 positions or which are substituted in some or all of these positions by fluorine.

In a more specific aspect the present invention provides 18,21-didesoxymacbecin analogues according to the formula (I) below, or a pharmaceutically acceptable salt thereof:

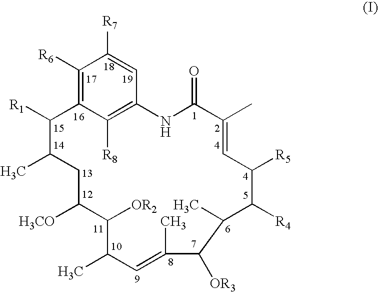

wherein:
R₁ represents H, OH, OMe;
R₂ represents H or Me;
R₃ represents H or CONH₂;
R₄ and R₅ either both represent H or together they represent a bond (i.e. C4 to C5 is a double bond);
R₆ represents H or F;
R₇ represents H or F; and
R₈ represents H or F.
18,21-didesoxymacbecin analogues are also referred to herein as “compounds of the invention”, such terms are used interchangeably herein.

The above structure shows a representative tautomer and the invention embraces all tautomers of the compounds of formula (I) for example keto compounds where enol compounds are illustrated and vice versa.

The invention embraces all stereoisomers of the compounds defined by structure (I) as shown above.

In a further aspect, the present invention provides 18,21-didesoxymacbecin analogues such as compounds of formula (I) or a pharmaceutically acceptable salt thereof, for use as a pharmaceutical.

### DEFINITIONS

The articles “a” and “an” are used herein to refer to one or to more than one (i.e. at least one) of the grammatical objects of the article. By way of example “an analogue” means one analogue or more than one analogue.

As used herein the term “analogue(s)” refers to chemical compounds that are structurally similar to another but which differ slightly in composition (as in the replacement of one atom by another or in the presence or absence of a particular functional group).

As used herein, the term “homologue(s)” refers a homologue of a gene or of a protein encoded by a gene disclosed herein from either an alternative macbecin biosynthetic cluster from a different macbecin producing strain or a homologue from an alternative ansamycin biosynthetic gene cluster e.g. from geldanamycin, herbimycin or reblastatin. Such homologue(s) encode a protein that performs the same function of can itself perform the same function as said gene or protein in the synthesis of macbecin or a related ansamycin polyketide. Preferably, such homologue(s) have at least 40% sequence identity, preferably at least 60%, at least 70%, at least 80%, at least 90% or at least 95% sequence identity to the sequence of the particular gene disclosed herein (Table 3, SEQ ID NO: 11 which is a sequence of all the genes in the cluster, from which the sequences of particular genes may be deduced). Percentage identity may be calculated using any program known to a person of skill in the art such as BLASTn or BLASTp, available on the NCBI website.

As used herein, the term “cancer” refers to a benign or malignant new growth of cells in skin or in body organs, for example but without limitation, breast, prostate, lung, kidney, pancreas, brain, stomach or bowel. A cancer tends to infiltrate into adjacent tissue and spread (metastasise) to distant organs, for example to bone, liver, lung or the brain. As used herein the term cancer includes both metastatic tumour cell types, such as but not limited to, melanoma, lymphoma, leukaemia, fibrosarcoma, rhabdomyosarcoma, and mastocytoma and types of tissue carcinoma, such as but not limited to, colorectal cancer, prostate cancer, small cell lung cancer and non-small cell lung cancer, breast cancer, pancreatic cancer, bladder cancer, renal cancer, gastric cancer, gliobastoma, primary liver cancer and ovarian cancer.

As used herein the term “B-cell malignancies” includes a group of disorders that include chronic lymphocytic leukaemia (CLL), multiple myeloma, and non-Hodgkin's lymphoma (NHL). They are neoplastic diseases of the blood and blood forming organs. They cause bone marrow and immune system dysfunction, which renders the host highly susceptible to infection and bleeding.

As used herein, the term “bioavailability” refers to the degree to which or rate at which a drug or other substance is absorbed or becomes available at the site of biological activity after administration. This property is dependent upon a number of factors including the solubility of the compound, rate of absorption in the gut, the extent of protein binding and metabolism etc. Various tests for bioavailability that would be familiar to a person of skill in the art are for example described in Egorin et al. (2002).

The term “water solubility” as used in this application refers to solubility in aqueous media, e.g. phosphate buffered saline (PBS) at pH 7.3. An exemplary water solubility assay is given in the Examples below.

The term “macbecin producing strain” as used in this application refers to strains, for example wild type strains as exemplified by A. pretiosum and A. mirum, which produce macbecin when cultured under suitable conditions, for example when fed the natural starter feed 3-amino-5-hydroxybenzoic acid.

As used herein the term “post-PKS genes(s)” refers to the genes required for post-polyketide synthase modifications of the polyketide, for example but without limitation monooxygenases, O-methyltransferases and carbamoyltransferases. Specifically, in the macbecin system these modifying genes include mbcM, mbcN, mbcP, mbcMT1, mbcMT2 and mbcP450.

As used herein the term “starter unit biosynthesis gene(s)” refers to the genes required for the production of the starter unit naturally incorporated, 3-amino-5-hydroxybenzoic acid (AHBA). Specifically, in the macbecin system these starter unit biosynthesis genes include AHk (AHBA kinase), Adh (aDHQ dehydrogenase), AHs (AHBA synthase), OX (oxidoreductase), PH (Phosphatase). Other strains that produce AHBA also contain AHBA biosynthesis genes.

The pharmaceutically acceptable salts of compounds of the invention such as the compounds of formula (I) include conventional salts formed from pharmaceutically acceptable inorganic or organic acids or bases as well as quaternary ammonium acid addition salts. More specific examples of suitable acid salts include hydrochloric, hydrobromic, sulfuric, phosphoric, nitric, perchloric, fumaric, acetic, propionic, succinic, glycolic, formic, lactic, maleic, tartaric, citric, palmoic, malonic, hydroxymaleic, phenylacetic, glutamic, benzoic, salicylic, fumaric, toluenesulfonic, methanesulfonic, naphthalene-2-sulfonic, benzenesulfonic hydroxynaphthoic, hydroiodic, malic, steroic, tannic and the like. Other acids such as oxalic, while not in themselves pharmaceutically acceptable, may be useful in the preparation of salts useful as intermediates in obtaining the compounds of the invention and their pharmaceutically acceptable salts. More specific examples of suitable basic salts include sodium, lithium, potassium, magnesium, aluminium, calcium, zinc, N,N′-dibenzylethylenediamine, chloroprocaine, choline, diethanolamine, ethylenediamine, N-methylglucamine and procaine salts. References hereinafter to a compound according to the invention include both compounds of formula (I) and their pharmaceutically acceptable salts.

As used herein the terms “18,21-dihydromacbecin” and “macbecin II” (the dihydroquinone form of macbecin) are used interchangeably.

### BRIEF DESCRIPTION OF THE DRAWINGS

FIG. 1: Representation of the biosynthesis of macbecin showing the first putative enzyme free intermediate, pre-macbecin and the post-PKS processing to macbecin. The list of PKS processing steps in the figure is not intended to represent the order of events. The following abbreviations are used for particular genes in the cluster: AL0—AHBA loading domain; ACP—Acyl Carrier Protein; KS-β-ketosynthase; AT—acyl transferase; DH—dehydratase; ER—enoyl reductase; KR-β-ketoreductase.

FIG. 2: Depiction of the sites of post-PKS processing of pre-macbecin to give macbecin.

FIG. 3: Diagrammatic representation of generation of the engineered strain BIOT-3806 in which plasmid pLSS308 was integrated into the chromosome by homologous recombination resulting in mbcM gene disruption.

FIG. 4: Diagrammatic representation of the construction of the in-frame deletion of mbcM described in example 2.

FIG. 5: A—shows the sequence of the PCR product PCRwv308, SEQ ID NO: 16

B—shows the sequence of the PCR product PCRwv309, SEQ ID NO: 19

FIG. 6: A—shows the DNA sequence resulting from the in-frame deletion of 502 amino acids in mbcM as described in example 3 (SEQ ID NO: 20 and 21),

Key: 1-21 bp encodes 3′ end of the phosphatase of 3-amino-5-hydroxybenzoic acid biosynthesis, 136-68 bp encodes mbcM deletion protein, 161-141 bp encodes 3′ end of mbcF.

B: shows the amino acid sequence of the protein (SEQ ID NO: 22). The protein sequence is generated from the complement strand shown in FIG. 6A.

FIG. 7: Diagrammatic representation of the generation of an Actinosynnema pretiosum strain in which the mbcP, mbcP450, mbcMT1 and mbcMT2 genes have been deleted in frame.

FIG. 8: Sequence of the amplified PCR product 1+2a (SEQ ID NO: 25)

FIG. 9: Sequence of the amplified PCR product 3b+4 (SEQ ID NO: 28)

FIG. 10: Structures of the compounds (14-20) produced in the Examples.

### DESCRIPTION OF THE INVENTION

The present invention provides 18,21-didesoxymacbecin analogues, as set out above, methods for the preparation of these compounds, methods for the use of these compounds in medicine and the use of these compounds as intermediates or templates for further semi-synthetic derivatisation or derivatisation by biotransformation methods.

Suitably R₁ represents H or OH. In one embodiment R₁ represents H. In another embodiment R₁ represents OH.

Suitably R₂ represents H.

Suitably R₃ represents CONH₂.

In one embodiment suitably R₄ and R₅ together represent a bond.

In another embodiment, suitably R₄ and R₅ each represent H.

In one example set of compounds R₆, R₇ and R₈ all represent hydrogen.

In another example set of compounds R₆, R₇ and R₈ do not all represent hydrogen.

In one embodiment, R₁ represents H, R₂ represents H, R₃ represents CONH₂ and R₄ and R₅ each represent H.

In another embodiment, R₁ represents OH, R₂ represents H, R₃ represents CONH₂ and R₄ and R₅ each represent H.

In one suitable embodiment of the invention R₁ represents H, R₂ represents H, R₃ represents CONH₂, R₄ and R₅ each represent H, R₆, R₇ and R₈ each represent H.

In one suitable embodiment of the invention R₁ represents OH, R₂ represents H, R₃ represents CONH₂, R₄ and R₅ each represent H, R₆, R₇ and R₈ each represent H.

In one suitable embodiment of the invention R₁ represents H, R₂ represents H, R₃ represents CONH₂, R₄ and R₅ each represent H, R₆ represents F and R₇ and R₈ each represent H.

In another suitable embodiment of the invention R₁ represents OH, R₂ represents H, R₃ represents CONH₂, R₄ and R₅ each represent H, R₆ represents F and R₇ and R₈ each represent H.

In another suitable embodiment of the invention R₁ represents H, R₂ represents H, R₃ represents CONH₂, R₄ and R₅ each represent H, R₆ represents H, R₇ represents F and R₈ represents H.

In another suitable embodiment of the invention R₁ represents OH, R₂ represents H, R₃ represents CONH₂, R₄ and R₅ each represent H, R₆ represents H, R₇ represents F and R₈ represents H.

In another suitable embodiment of the invention R₁ represents H, R₂ represents H, R₃ represents CONH₂, R₄ and R₅ each represent H, R₆ and R₇ each represent F and R₈ represents H, for example as represented in the following structure,

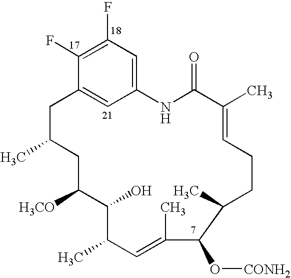

In another suitable embodiment of the invention R₁ represents OH, R₂ represents H, R₃ represents CONH₂, R₄ and R₅ each represent H, R₆ and R₇ each represent F and R₈ represents H.

In another suitable embodiment of the invention R₁ represents H, R₂ represents H, R₃ represents CONH₂, R₄ and R₅ each represent H, R₆, R₇ and R₈ each represent F.

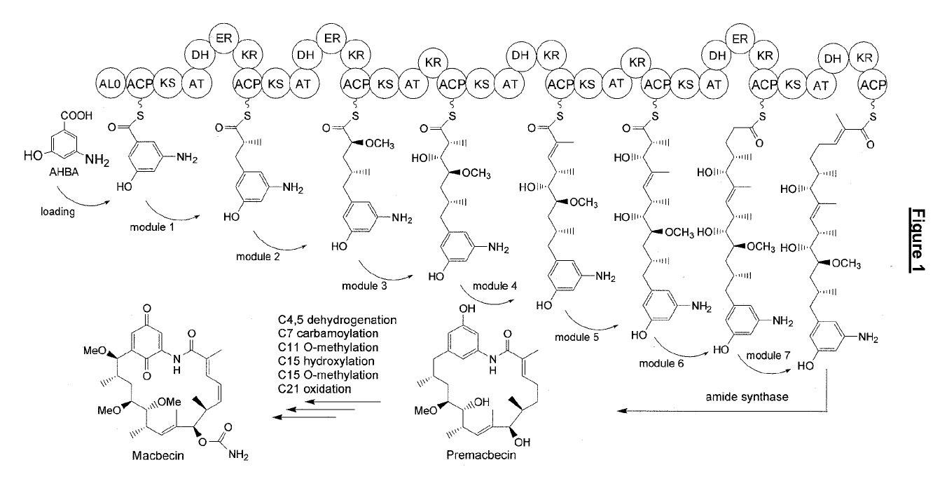

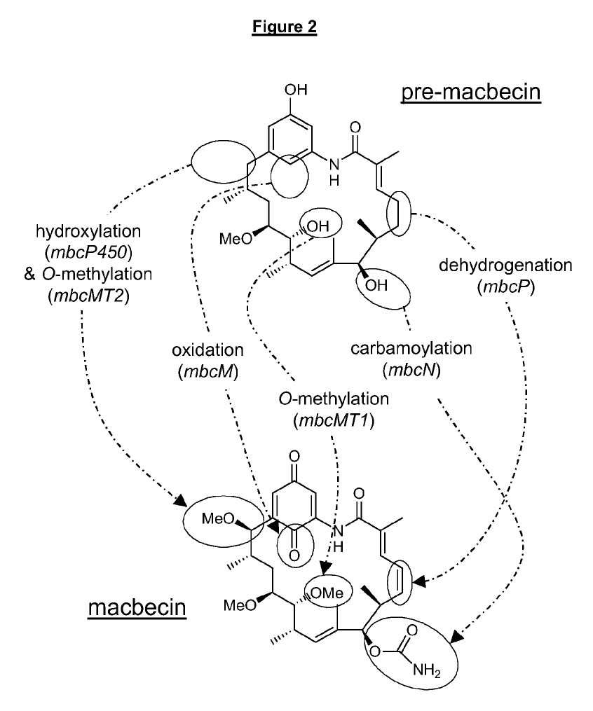

The preferred stereochemistry of the non-hydrogen sidechains to the ansa ring is as shown in FIGS. 1 and 2 below (that is to say the preferred stereochemistry follows that of macbecin).

The present invention also provides for the use of an 18,21-didesoxymacbecin analogue as a substrate for further modification either by biotransformation or by synthetic chemistry.

In one aspect the present invention provides an 18,21-didesoxymacbecin analogue for use as a medicament. In a further embodiment the present invention provides an 18,21-didesoxymacbecin analogue for use in the treatment of cancer, B-cell malignancies, malaria, fungal infection, diseases of the central nervous system and neurodegenerative diseases, diseases dependent on angiogenesis, autoimmune diseases and/or as a prophylactic pre-treatment for cancer.

In another aspect the present invention provides for the use of an 18,21-didesoxymacbecin analogue in the manufacture of a medicament. In a further embodiment the present invention provides for the use of an 18,21-didesoxymacbecin analogue in the manufacture of a medicament for the treatment of cancer, B-cell malignancies, malaria, fungal infection, diseases of the central nervous system and neurodegenerative diseases, diseases dependent on angiogenesis, autoimmune diseases and/or as a prophylactic pre-treatment for cancer.

In a further embodiment the present invention provides a method of treatment of cancer, B-cell malignancies, malaria, fungal infection, diseases of the central nervous system and neurodegenerative diseases, diseases dependent on angiogenesis, autoimmune diseases and/or a prophylactic pre-treatment for cancer, said method comprising administering to a patient in need thereof a therapeutically effective amount of an 18,21-didesoxymacbecin analogue.

As noted above, compounds of the invention may be expected to be useful in the treatment of cancer and/or B-cell malignancies. Compounds of the invention may also be effective in the treatment of other indications for example, but not limited to malaria, fungal infection, diseases of the central nervous system and neurodegenerative diseases, diseases dependent on angiogenesis, autoimmune diseases such as rheumatoid arthritis or as a prophylactic pre-treatment for cancer.

Diseases of the central nervous system and neurodegenerative diseases include, but are not limited to, Alzheimer's disease, Parkinson's disease, Huntington's disease, prion diseases, spinal and bulbar muscular atrophy (SBMA) and amyotrophic lateral sclerosis (ALS).

Diseases dependent on angiogenesis include, but are not limited to, age-related macular degeneration, diabetic retinopathy and various other ophthalmic disorders, atherosclerosis and rheumatoid arthritis.

Autoimmune diseases include, but are not limited to, rheumatoid arthritis, multiple sclerosis, type I diabetes, systemic lupus erythematosus and psoriasis,

“Patient” embraces human and other animal (especially mammalian) subjects, preferably human subjects. Accordingly the methods and uses of the 18,21-didesoxymacbecin analogues of the invention are of use in human and veterinary medicine, preferably human medicine.

The aforementioned compounds of the invention or a formulation thereof may be administered by any conventional method for example but without limitation they may be administered parenterally (including intravenous administration), orally, topically (including buccal, sublingual or transdermal), via a medical device (e.g. a stent), by inhalation, or via injection (subcutaneous or intramuscular). The treatment may consist of a single dose or a plurality of doses over a period of time.

Whilst it is possible for a compound of the invention to be administered alone, it is preferable to present it as a pharmaceutical formulation, together with one or more acceptable diluents or carriers. Thus there is provided a pharmaceutical composition comprising a compound of the invention together with one or more pharmaceutically acceptable diluents or carriers. The diluents(s) or carrier(s) must be “acceptable” in the sense of being compatible with the compound of the invention and not deleterious to the recipients thereof. Examples of suitable carriers are described in more detail below.

The compounds of the invention may be administered alone or in combination with other therapeutic agents. Co-administration of two (or more) agents may allow for significantly lower doses of each to be used, thereby reducing the side effects seen. It might also allow resensitisation of a disease, such as cancer, to the effects of a prior therapy to which the disease has become resistant. There is also provided a pharmaceutical composition comprising a compound of the invention and a further therapeutic agent together with one or more pharmaceutically acceptable diluents or carriers.

In a further aspect, the present invention provides for the use of a compound of the invention in combination therapy with a second agent eg a second agent for the treatment of cancer or B-cell malignancies such as a cytotoxic or cytostatic agent.

In one embodiment, a compound of the invention is co-administered with another therapeutic agent e.g. a therapeutic agent such as a cytotoxic or cytostatic agent for the treatment of cancer or B-cell malignancies. Exemplary further agents include cytotoxic agents such as alkylating agents and mitotic inhibitors (including topoisomerase II inhibitors and tubulin inhibitors). Other exemplary further agents include DNA binders, antimetabolites and cytostatic agents such as protein kinase inhibitors and tyrosine kinase receptor blockers. Suitable agents include, but are not limited to, methotrexate, leukovorin, prenisone, bleomycin, cyclophosphamide, 5-fluorouracil, paclitaxel, docetaxel, vincristine, vinblastine, vinorelbine, doxorubicin (adriamycin), tamoxifen, toremifene, megestrol acetate, anastrozole, goserelin, anti-HER2 monoclonal antibody (e.g. trastuzumab, trade name Herceptin™), capecitabine, raloxifene hydrochloride, EGFR inhibitors (e.g. gefitinib, trade name Iressa®, erlotinib, trade name Tarceva™, cetuximab, trade name Erbitux™), VEGF inhibitors (e.g. bevacizumab, trade name Avastin™) and proteasome inhibitors (e.g. bortezomib, trade name Velcade™). Further suitable agents include, but are not limited to, conventional chemotherapeutics such as cisplatin, cytarabine, cyclohexylchloroethylnitrosurea, gemcitabine, Ifosfamid, leucovorin, mitomycin, mitoxantone, oxaliplatin, taxanes including taxol and vindesine; hormonal therapies monoclonal antibody therapies such as cetuximab (anti-EGFR); protein kinase inhibitors such as dasatinib, lapatinib; histone deacetylase (HDAC) inhibitors such as vorinostat; angiogenesis inhibitors such as sunitinib, sorafenib, lenalidomide; mTOR inhibitors such as temsirolimus; and imatinib, trade name Glivec®. Additionally, a compound of the invention may be administered in combination with other therapies including, but not limited to, radiotherapy or surgery.

The formulations may conveniently be presented in unit dosage form and may be prepared by any of the methods well known in the art of pharmacy. Such methods include the step of bringing into association the active ingredient (compound of the invention) with the carrier which constitutes one or more accessory ingredients. In general the formulations are prepared by uniformly and intimately bringing into association the active ingredient with liquid carriers or finely divided solid carriers or both, and then, if necessary, shaping the product.

The compounds of the invention will normally be administered orally or by any parenteral route, in the form of a pharmaceutical formulation comprising the active ingredient, optionally in the form of a non-toxic organic, or inorganic, acid, or base, addition salt, in a pharmaceutically acceptable dosage form. Depending upon the disorder and patient to be treated, as well as the route of administration, the compositions may be administered at varying doses.

For example, the compounds of the invention can be administered orally, buccally or sublingually in the form of tablets, capsules, ovules, elixirs, solutions or suspensions, which may contain flavouring or colouring agents, for immediate-, delayed- or controlled-release applications.

Such tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropylmethylcellulose (HPMC), hydroxy-propylcellulose (HPC), sucrose, gelatine and acacia. Additionally, lubricating agents such as magnesium stearate, stearic acid, glyceryl behenate and talc may be included.

Solid compositions of a similar type may also be employed as fillers in gelatine capsules. Preferred excipients in this regard include lactose, starch, a cellulose, milk sugar or high molecular weight polyethylene glycols. For aqueous suspensions and/or elixirs, the compounds of the invention may be combined with various sweetening or flavouring agents, colouring matter or dyes, with emulsifying and/or suspending agents and with diluents such as water, ethanol, propylene glycol and glycerine, and combinations thereof.

A tablet may be made by compression or moulding, optionally with one or more accessory ingredients. Compressed tablets may be prepared by compressing in a suitable machine the active ingredient in a free-flowing form such as a powder or granules, optionally mixed with a binder (e.g. povidone, gelatine, hydroxypropylmethyl cellulose), lubricant, inert diluent, preservative, disintegrant (e.g. sodium starch glycolate, cross-linked povidone, cross-linked sodium carboxymethyl cellulose), surface-active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein using, for example, hydroxypropylmethylcellulose in varying proportions to provide desired release profile.

Formulations in accordance with the present invention suitable for oral administration may be presented as discrete units such as capsules, cachets or tablets, each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Formulations suitable for topical administration in the mouth include lozenges comprising the active ingredient in a flavoured basis, usually sucrose and acacia or tragacanth; pastilles comprising the active ingredient in an inert basis such as gelatine and glycerine, or sucrose and acacia; and mouth-washes comprising the active ingredient in a suitable liquid carrier.

It should be understood that in addition to the ingredients particularly mentioned above the formulations of this invention may include other agents conventional in the art having regard to the type of formulation in question, for example those suitable for oral administration may include flavouring agents.

Pharmaceutical compositions adapted for topical administration may be formulated as ointments, creams, suspensions, lotions, powders, solutions, pastes, gels, impregnated dressings, sprays, aerosols or oils, transdermal devices, dusting powders, and the like. These compositions may be prepared via conventional methods containing the active agent. Thus, they may also comprise compatible conventional carriers and additives, such as preservatives, solvents to assist drug penetration, emollient in creams or ointments and ethanol or oleyl alcohol for lotions. Such carriers may be present as from about 1% up to about 98% of the composition. More usually they will form up to about 80% of the composition. As an illustration only, a cream or ointment is prepared by mixing sufficient quantities of hydrophilic material and water, containing from about 5-10% by weight of the compound, in sufficient quantities to produce a cream or ointment having the desired consistency.

Pharmaceutical compositions adapted for transdermal administration may be presented as discrete patches intended to remain in intimate contact with the epidermis of the recipient for a prolonged period of time. For example, the active agent may be delivered from the patch by iontophoresis.

For applications to external tissues, for example the mouth and skin, the compositions are preferably applied as a topical ointment or cream. When formulated in an ointment, the active agent may be employed with either a paraffinic or a water-miscible ointment base.

Alternatively, the active agent may be formulated in a cream with an oil-in-water cream base or a water-in-oil base.

For parenteral administration, fluid unit dosage forms are prepared utilizing the active ingredient and a sterile vehicle, for example but without limitation water, alcohols, polyols, glycerine and vegetable oils, water being preferred. The active ingredient, depending on the vehicle and concentration used, can be either suspended or dissolved in the vehicle. In preparing solutions the active ingredient can be dissolved in water for injection and filter sterilised before filling into a suitable vial or ampoule and sealing.

Advantageously, agents such as local anaesthetics, preservatives and buffering agents can be dissolved in the vehicle. To enhance the stability, the composition can be frozen after filling into the vial and the water removed under vacuum. The dry lyophilized powder is then sealed in the vial and an accompanying vial of water for injection may be supplied to reconstitute the liquid prior to use.

Parenteral suspensions are prepared in substantially the same manner as solutions, except that the active ingredient is suspended in the vehicle instead of being dissolved and sterilization cannot be accomplished by filtration. The active ingredient can be sterilised by exposure to ethylene oxide before suspending in the sterile vehicle. Advantageously, a surfactant or wetting agent is included in the composition to facilitate uniform distribution of the active ingredient.

The compounds of the invention may also be administered using medical devices known in the art. For example, in one embodiment, a pharmaceutical composition of the invention can be administered with a needleless hypodermic injection device, such as the devices disclosed in U.S. Pat. No. 5,399,163; U.S. Pat. No. 5,383,851; U.S. Pat. No. 5,312,335; U.S. Pat. No. 5,064,413; U.S. Pat. No. 4,941,880; U.S. Pat. No. 4,790,824; or U.S. Pat. No. 4,596,556. Examples of well-known implants and modules useful in the present invention include: U.S. Pat. No. 4,487,603, which discloses an implantable micro-infusion pump for dispensing medication at a controlled rate; U.S. Pat. No. 4,486,194, which discloses a therapeutic device for administering medicaments through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Many other such implants, delivery systems, and modules are known to those skilled in the art.

The dosage to be administered of a compound of the invention will vary according to the particular compound, the disease involved, the subject, and the nature and severity of the disease and the physical condition of the subject, and the selected route of administration. The appropriate dosage can be readily determined by a person skilled in the art.

The compositions may contain from 0.1% by weight, preferably from 5-60%, more preferably from 10-30% by weight, of a compound of invention, depending on the method of administration.

It will be recognized by one of skill in the art that the optimal quantity and spacing of individual dosages of a compound of the invention will be determined by the nature and extent of the condition being treated, the form, route and site of administration, and the age and condition of the particular subject being treated, and that a physician will ultimately determine appropriate dosages to be used. This dosage may be repeated as often as appropriate. If side effects develop the amount and/or frequency of the dosage can be altered or reduced, in accordance with normal clinical practice.

In a further aspect the present invention provides methods for the production of 18,21-didesoxymacbecin analogues.

Macbecin can be considered to be biosynthesised in two stages. In the first stage the core-PKS genes assemble the macrolide core by the repeated assembly of simple carboxylic acid precursors to give a polyketide chain which is then cyclised to form the first enzyme-free intermediate “pre-macbecin”, see FIG. 1. In the second stage a series of “post-PKS” tailoring enzymes (e.g. P450 monooxygenases, methyltransferases, FAD-dependent oxygenases and a carbamoyltransferase) act to add the various additional groups to the pre-macbecin template resulting in the final parent compound structure, see FIG. 2. The 18,21-didesoxymacbecin analogues may be biosynthesised in a similar manner.

This biosynthetic production may be exploited by biotransformation optionally combined with genetic engineering of suitable producer strains to result in the production of novel compounds. In particular, the present invention provides a method of producing 18,21-didesoxymacbecin analogues said method comprising:
a) providing a first host strain that produces macbecin or an analogue when cultured under appropriate conditions
b) feeding a non-natural starter acid to said strain
c) culturing said strain under suitable conditions for the production of 18,21-didesoxymacbecin analogues; and
d) optionally isolating the compounds produced.
   The method may additionally comprise the step of:
e) deleting or inactivating one or more of the starter unit biosynthesis genes, or a homologue thereof, said step usually occurring prior to step c) and/or the method may additionally comprise the step of:
f) deleting or inactivating one or more post-PKS genes, said step usually occurring prior to step c).

In step (a) by “a host strain that produces macbecin or an analogue thereof” is meant a strain that produces macbecin or those analogues of macbecin particularly 18,21-didesoxymacbecin analogues that are embraced by the definitions of R₁-R₈ when cultured under appropriate conditions. Appropriate conditions (and suitable conditions in step (c)) include the provision of a suitable starter feed and growth media of suitable composition (which will be known to a skilled person or may be determined by methods known per se).

Suitably the non-natural starter feed is a substituted benzoic acid (not being 3-amino-5-hydroxy-benzoic acid which is the natural starter acid). Most suitably the non-natural starter feed is 3-amino-benzoic acid wherein the benzene ring is optionally substituted by one to three fluorine atoms.

In a suitable embodiment the non-natural starter acid feed is 3-aminobenzoic acid.

In another suitable embodiment the non-natural starter acid feed is 5-amino-2-fluorobenzoic acid.

In another suitable embodiment the non-natural starter acid feed is 5-amino-3-fluorobenzoic acid.

In another suitable embodiment the non-natural starter acid feed is 5-amino-2,3-di-fluorobenzoic acid.

In another suitable embodiment the non-natural starter acid feed is 5-amino-2,3,6-tri-fluorobenzoic acid.

One skilled in the art will appreciate that there are alternative non-natural starter units that could be fed to the host strain to produce the same compound(s) for example, but not limited to, the methyl ester, the ethyl ester, the N-acetyl-cysteamine thioester of the substituted benzoic acid and the diketide analogue of the biosynthetic intermediate activated appropriately for incorporation for example as the N-acetyl-cysteamine thioester.

In a first embodiment of the invention the host strain is a macbecin producing strain.

In an alternative embodiment, the host strain is an engineered strain based on a macbecin producing strain in which one or more of the starter unit biosynthetic genes have been deleted or inactivated.

In a further embodiment the host strain is an engineered strain based on a macbecin producing strain in which one or more of the post-PKS genes have been deleted or inactivated. For example, the host strain may be an engineered strain based on a macbecin producing strain in which mbcM and optionally further post-PKS genes have been deleted or inactivated. Specifically, the host strain may be an engineered strain based on a macbecin producing strain in which mbcM has been deleted or inactivated. Alternatively the host strain may be an engineered strain based on a macbecin producing strain in which mbcM, mbcMT1, mbcMT2, mbcP and mbcP450 have been deleted or inactivated.

Suitably the one or more starter unit biosynthetic genes and/or post-PKS genes will be deleted or inactivated selectively.

In a further embodiment, one or more starter unit biosynthetic genes or post-PKS genes are inactivated in said engineered strain by integration of DNA into the gene(s) such that functional protein is not produced. In an alternative embodiment, one or more of said starter unit biosynthetic genes or post-PKS genes are deleted in said engineered strain by making a targeted deletion or deletions. In a further embodiment one or more starter unit biosynthetic genes or post-PKS genes are inactivated in said engineered strain by site-directed mutagenesis. In a further embodiment a macbecin producing host strain is subjected to mutagenesis, chemical or UV, and a modified strain is selected in which one or more of the starter unit biosynthetic enzymes or post-PKS enzymes are not functional. The present invention also encompasses mutations of the regulators controlling the expression of one or more of the starter unit biosynthetic genes or post-PKS genes, a person of skill in the art will appreciate that deletion or inactivation of a regulator may have the same outcome as deletion or inactivation of the gene.

In a further embodiment an engineered strain in which one or more post-PKS genes have been deleted or inactivated as above, has re-introduced into it one or more of the same post PKS genes, or homologues thereof from an alternative macbecin producing strain.

In a further embodiment an engineered strain in which one or more genes has been deleted or inactivated is complemented by one or more of the post PKS genes from a heterologous PKS cluster including, but not limited to the clusters directing the biosynthesis of rifamycin, ansamitocin, geldanamycin or herbimycin.

A method of selectively deleting or inactivating a post PKS gene comprises:
(i) designing degenerate oligos based on homologue(s) of the gene of interest (e.g. from the rifamycin, geldanamycin or herbimycin biosynthetic clusters and/or other available sequences) and isolating the internal fragment of the gene of interest from a suitable macbecin producing strain by using these primers in a PCR reaction,
(ii) integrating a plasmid containing this fragment into either the same, or a different macbecin producing strain followed by homologous recombination, which results in the disruption of the targeted gene,
(iii) culturing the strain thus produced under conditions suitable for the production of the macbecin analogues, i.e. 18,21-didesoxymacbecin analogues.

In a specific embodiment, the macbecin-producing strain in step (i) is Actinosynnema mirum (A. mirum). In a further specific embodiment the macbecin-producing strain in step (ii) is Actinosynnema pretiosum (A. pretiosum).

A person of skill in the art will appreciate that an equivalent strain may be achieved using alternative methods to that described above, e.g.:
Degenerate oligos may be used to amplify the gene of interest from any macbecin producing strain for example, but not limited to A. pretiosum, or A. mirum
Different degenerate oligos may be designed which will successfully amplify an appropriate region of the post-PKS gene, or a homologue thereof, from a macbecin producer, or strain producing a homologue thereof.
The sequence of the gene of the A. pretiosum strain may be used to generate the oligos which may be specific to the gene of A. pretiosum and then the internal fragment may be amplified from any macbecin producing strain e.g A. pretiosum or A. mirum.
The sequence of the gene of the A. pretiosum strain may be used along with the sequence of homologous genes to generate degenerate oligos to the gene of A. pretiosum and then the internal fragment may be amplified from any macbecin producing strain e.g A. pretiosum or A. mirum.

FIG. 2 shows the activity of the post-PKS genes in the macbecin biosynthetic cluster. A person of skill in the art would thus be able to identify which additional post-PKS genes would need to be deleted or inactivated in order to arrive at a strain that will produce the compound(s) of interest.

It may be observed in these systems that when a strain is generated in which one or more of the post-PKS genes does not function as a result of one of the methods described including inactivation or deletion, that more than one 18,21-didesoxymacbecin analogue may be produced. There are a number of possible reasons for this which will be appreciated by those skilled in the art. For example there may be a preferred order of post-PKS steps and removing a single activity leads to all subsequent steps being carried out on substrates that are not natural to the enzymes involved. This can lead to intermediates building up in the culture broth due to a lowered efficiency towards the novel substrates presented to the post-PKS enzymes, or to shunt products which are no longer substrates for the remaining enzymes possibly because the order of steps has been altered.

A person of skill in the art will appreciate that the ratio of compounds observed in a mixture can be manipulated by using variations in the growth conditions.

One skilled in the art will appreciate that in a biosynthetic cluster some genes are organised in operons and disruption of one gene will often have an effect on expression of subsequent genes in the same operon.

When a mixture of compounds is observed these can be readily separated using standard techniques some of which are described in the following examples.

18,21-Didesoxymacbecin analogues may be screened by a number of methods, as described herein, and in the circumstance where a single compound shows a favourable profile a strain can be engineered to make this compound preferably. In the unusual circumstance when this is not possible, an intermediate can be generated which is then biotransformed to produce the desired compound.

The present invention provides novel macbecin analogues generated by the selected deletion or inactivation of one or more post-PKS genes from the macbecin PKS gene cluster. In particular, the present invention relates to novel 18,21-didesoxymacbecin analogues produced by feeding a non-natural starter unit to a macbecin producing strain, optionally combined with the selected deletion or inactivation of one or more post-PKS genes, from the macbecin PKS gene cluster.

A person of skill in the art will appreciate that a gene does not need to be completely deleted for the gene product to be rendered non-functional, consequentially the term “deleted or inactivated” as used herein encompasses any method by which the gene product is rendered non-functional including but not limited to: deletion of the gene in its entirety, deletion of part of the gene, inactivation by insertion into the target gene, site-directed mutagenesis which results in the gene either not being expressed or being expressed to produce inactive protein, mutagenesis of the host strain which results in the gene either not being expressed or being expressed to produce inactive protein (e.g. by radiation or exposure to mutagenic chemicals, protoplast fusion or transposon mutagenesis). Alternatively the function of an active gene product can be impaired chemically with inhibitors, for example metapyrone (alternative name 2-methyl-1,2-di(3-pyridyl-1-propanone), EP 0 627 009) and ancymidol are inhibitors of oxygenases and these compounds can be added to the production medium to generate analogues. Additionally, sinefungin is a methyl transferase inhibitor that can be used similarly but for the inhibition of methyl transferase activity in vivo (McCammon and Parks, 1981).

In an alternative embodiment, all of the post-PKS genes may be deleted or inactivated and then one or more of the genes may then be reintroduced by complementation (e.g. at an attachment site, on a self-replicating plasmid or by insertion into a homologous region of the chromosome). Therefore, in a particular embodiment the present invention relates to methods for the generation of 18,21-didesoxymacbecin analogues, said method comprising:
a) providing a first host strain that produces macbecin when cultured under appropriate conditions
b) optionally selectively deleting or inactivating all the post-PKS genes,
c) feeding a non-natural starter unit to said strain
d) culturing said modified host strain under suitable conditions for the production of 18,21-didesoxymacbecin analogues; and
e) optionally isolating the compounds produced.

In an alternative embodiment, one or more of the deleted post-PKS genes are reintroduced. In a further embodiment, 1 or more of the post-PKS genes selected from the group consisting of mbcM, mbcN, mbcP, mbcMT1, mbcMT2 and mbcP450 are reintroduced. In a further embodiment, 2 or more of the post-PKS genes selected from the group consisting of mbcM, mbcN, mbcP, mbcMT1, mbcMT2 and mbcP450 are reintroduced. In a further embodiment, 3 or more of the post-PKS genes selected from the group consisting of mbcM, mbcN, mbcP, mbcMT1, mbcMT2 and mbcP450 are reintroduced. In a further embodiment, 4 or more of the post-PKS genes selected from the group consisting of mbcM, mbcN, mbcP, mbcMT1, mbcMT2 and mbcP450 are reintroduced. In a further alternative embodiment, 5 or more of the post-PKS genes selected from the group consisting of mbcM, mbcN, mbcP, mbcMT1, mbcMT2 and mbcP450 are reintroduced. Optionally genes from other PKS biosynthetic clusters such as but not limited to the geldanamycin or herbimycin pathways can be introduced appropriately.

Additionally, it will be apparent to a person of skill in the art that a subset of the post-PKS genes, could be deleted or inactivated and optionally a smaller subset of said post-PKS genes could be reintroduced to arrive at a strain that, when fed a non-natural starter unit, produces 18,21-didesoxymacbecin analogues.

Therefore, in a preferred embodiment the present invention relates to methods for the generation of 18,21-didesoxymacbecin analogues, said method comprising:
a) providing a first host strain that produces macbecin when cultured under appropriate conditions
b) selectively deleting or inactivating mbcM,
c) feeding a non-natural starter unit to said strain
d) culturing said modified host strain under suitable conditions for the production of 18,21-didesoxymacbecin analogues; and
e) optionally isolating the compounds produced.

In a further preferred embodiment the present invention relates to methods for the generation of 18,21-didesoxymacbecin analogues, said method comprising:
a) providing a first host strain that produces macbecin when cultured under appropriate conditions
b) selectively deleting or inactivating mbcM and mbcP450
c) optionally selectively deleting or inactivating further post-PKS genes
d) feeding a non-natural starter unit to said strain
e) culturing said modified host strain under suitable conditions for the production of 18,21-didesoxymacbecin analogues; and
f) optionally isolating the compounds produced.

In a further preferred embodiment the present invention relates to methods for the generation of 18,21-didesoxymacbecin analogues, said method comprising:
a) providing a first host strain that produces macbecin when cultured under appropriate conditions
b) selectively deleting or inactivating mbcM, mbcMT1, mbcMT2, mbcP and mbcP450
c) optionally selectively deleting or inactivating further post-PKS genes or starter unit biosynthesis genes
d) feeding a non-natural starter unit to said strain
e) culturing said modified host strain under suitable conditions for the production of 18,21-didesoxymacbecin analogues; and
f) optionally isolating the compounds produced.

It is well known to those skilled in the art that polyketide gene clusters may be expressed in heterologous hosts (Pfeifer and Khosla, 2001). Accordingly, the present invention includes the transfer of the macbecin biosynthetic gene cluster, with or without resistance and regulatory genes, either otherwise complete or containing deletions, into a heterologous host. Alternatively, the complete macbecin biosynthetic cluster can be transferred into a heterologous host, with or without resistance and regulatory genes, and it can then be manipulated by the methods described herein to delete or inactivate one or more of the post-PKS genes or starter unit biosynthesis genes. Methods and vectors for the transfer as defined above of such large pieces of DNA are well known in the art (Rawlings, 2001; Staunton and Weissman, 2001) or are provided herein in the methods disclosed. In this context a preferred host cell strain is a prokaryote, more preferably an actinomycete or Escherichia coli, still more preferably include, but are not limited to Actinosynnema mirum (A. mirum), Actinosynnema pretiosum subsp. pretiosum (A. pretiosum), S. hygroscopicus, S. hygroscopicus sp., S. hygroscopicus var. ascomyceticus, Streptomyces tsukubaensis, Streptomyces coelicolor, Streptomyces lividans, Saccharopolyspora erythraea, Streptomyces fradiae, Streptomyces avermitilis, Streptomyces cinnamonensis, Streptomyces rimosus, Streptomyces albus, Streptomyces griseofuscus, Streptomyces longisporoflavus, Streptomyces venezuelae, Streptomyces albus, Micromonospora sp., Micromonospora griseorubida, Amycolatopsis mediterranei or Actinoplanes sp. N902-109. Further examples include Streptomyces hygroscopicus subsp. geldanus and Streptomyces violaceusniger.

In one embodiment the entire biosynthetic cluster is transferred. In an alternative embodiment the entire PKS is transferred without any of the associated starter unit biosynthesis genes and/or post-PKS genes.

In a further embodiment the entire macbecin biosynthetic cluster is transferred and then manipulated according to the description herein.

In an alternative aspect of the invention, the 18,21-didesoxymacbecin analogue(s) of the present invention may be further processed by biotransformation with an appropriate strain. The appropriate strain either being an available wild type strain for example, but without limitation Actinosynnema mirum, Actinosynnema pretiosum subsp. pretiosum, S. hygroscopicus, S. hygroscopicus sp. Alternatively, an appropriate strain may be engineered to allow biotransformation with particular post-PKS enzymes for example, but without limitation, those encoded by mbcM, mbcN, mbcP, mbcMT2, mbcP450 (as defined herein), gdmN, gdmM, gdmL, gdmP, (Rascher et al., 2003) the geldanamycin O-methyl transferase, hbmN, hbmL, hbmP, (Rascher et al., 2005) herbimycin O-methyl transferases and further herbimycin mono-oxygenases, asm7, asm10, asm11, asm12, asm19 and asm21 (Cassady et al., 2004, Spiteller et al., 2003). Where genes have yet to be identified or the sequences are not in the public domain it is routine to those skilled in the art to acquire such sequences by standard methods. For example the sequence of the gene encoding the geldanamycin O-methyl transferase is not in the public domain, but one skilled in the art could generate a probe, either a heterologous probe using a similar O-methyl transferase, or a homologous probe by designing degenerate primers from available homologous genes to carry out Southern blots on a geldanamycin producing strain and thus acquire this gene to generate biotransformation systems.

In a particular embodiment the strain may have had one or more of its native polyketide clusters deleted, either entirely or in part, or otherwise inactivated, so as to prevent the production of the polyketide produced by said native polyketide cluster. Said engineered strain may be selected from the group including, for example but without limitation, Actinosynnema mirum, Actinosynnema pretiosum subsp. pretiosum, S. hygroscopicus, S. hygroscopicus sp., S. hygroscopicus var. ascomyceticus, Streptomyces tsukubaensis, Streptomyces coelicolor, Streptomyces lividans, Saccharopolyspora erythraea, Streptomyces fradiae, Streptomyces avermitilis, Streptomyces cinnamonensis, Streptomyces rimosus, Streptomyces albus, Streptomyces griseofuscus, Streptomyces longisporoflavus, Streptomyces venezuelae, Micromonospora sp., Micromonospora griseorubida, Amycolatopsis mediterranei or Actinoplanes sp. N902-109. Further possible strains include Streptomyces hygroscopicus subsp. geldanus and Streptomyces violaceusniger.

Although the process for preparation of the 18,21-didesoxymacbecin analogues of the invention as described above is substantially or entirely biosynthetic, it is not ruled out to produce or interconvert 18,21-didesoxymacbecin analogues of the invention by a process which comprises standard synthetic chemical methods.

In order to allow for the genetic manipulation of the macbecin PKS gene cluster, first the gene cluster was sequenced from Actinosynnema pretiosum subsp. pretiosum however, a person of skill in the art will appreciate that there are alternative strains which produce macbecin, for example but without limitation Actinosynnema mirum. The macbecin biosynthetic gene cluster from these strains may be sequenced as described herein for Actinosynnema pretiosum subsp. pretiosum, and the information used to generate equivalent strains.

Further aspects of the invention include:
An engineered strain based on a macbecin producing strain in which mbcM and optionally further post-PKS genes have been deleted or inactivated, particularly such an engineered strain in which mbcM has been deleted or inactivated or such an engineered strain in which mbcM, mbcMT1, mbcMT2, mbcP and mbcP450 have been deleted or inactivated. Suitably the macbecin producing strain is A pretiosum or A mirum.
An engineered strain based on a macbecin producing strain in which mbcM and optionally further post-PKS genes and/or starter unit biosynthesis genes have been deleted or inactivated, particularly such an engineered strain in which mbcM has been deleted or inactivated or such an engineered strain in which mbcM, mbcMT1, mbcMT2, mbcP and mbcP450 have been deleted or inactivated. Suitably the macbecin producing strain is A pretiosum or A mirum.
Use of such an engineered strain in the preparation of a 18,21-didesoxymacbecin analogue.

Compounds of the invention are advantageous in that they may be expected to have one or more of the following properties: tight binding to Hsp90, fast on-rate of binding to Hsp90, good activity against one or more different cancer sub-types compared with the parent compound; good toxicological profile such as good hepatotoxicity profile, good nephrotoxicity, good cardiac safety; good water solubility; good metabolic stability; good formulation ability; good bioavailability; good pharmacokinetic or pharmacodynamic properties such as tight binding to Hsp90, fast on-rate of binding to Hsp90 and/or good brain pharmacokinetics; good cell uptake; and low binding to erythrocytes.

### EXAMPLES

### General Methods

### Fermentation of Cultures

Conditions used for growing the bacterial strains Actinosynnema pretiosum subsp. pretiosum ATCC 31280 (U.S. Pat. No. 4,315,989) and Actinosynnema mirum DSM 43827 (KCC A-0225, Watanabe et al., 1982) were described in the U.S. Pat. Nos. 4,315,989 and 4,187,292. Methods used herein were adapted from these and are as follows for culturing of broths in tubes or flasks in shaking incubators, variations to the published protocols are indicated in the examples. Strains were grown on ISP2 agar (Medium 3, Shirling, E. B. and Gottlieb, D., 1966) at 28° C. for 2-3 days and used to inoculate seed medium (Medium 1, see below and U.S. Pat. No. 4,315,989 and U.S. Pat. No. 4,187,292). The inoculated seed medium was then incubated with shaking between 200 and 300 rpm with a 5 or 2.5 cm throw at 28° C. for 48 h. For production of macbecin, 18,21-dihydromacbecin and macbecin analogues such as 18,21-didesoxymacbecin analogues the fermentation medium (Medium 2, see below and U.S. Pat. No. 4,315,989 and U.S. Pat. No. 4,187,292) was inoculated with 2.5%-10% of the seed culture and incubated with shaking between 200 and 300 rpm with a 5 or 2.5 cm throw at 26° C. for six days except where otherwise indicated in the examples. The culture was then harvested for extraction.

### Media

### Medium 1—Seed Medium

In 1 L of distilled water

| | | |
|---|---|---|
| Glucose | 20 | g |
| Soluble potato starch (Sigma) | 30 | g |
| Spray dried corn steep liquor (Roquette Freres) | 10 | g |
| ‘Nutrisoy’ toasted soy flour (Archer Daniels Midland) | 10 | g |
| Peptone from milk solids (Sigma) | 5 | g |
| NaCl | 3 | g |
| CaCO₃ | 5 | g |

| | |
|---|---|
| Adjust pH with NaOH | 7.0 |

Sterilsation by autoclaving at 121° C. for 20 minutes.
Apramycin was added when appropriate after autoclaving to give a final concentration of 50 mg/L.

### Medium 2—Fermentation Medium

In 1 L of distilled water

| | | |
|---|---|---|
| Glycerol | 50 | g |
| Spray dried corn steep liquor (Roquette Freres) | 10 | g |
| ‘Bacto’ yeast extract (Difco) | 20 | g |
| KH₂PO₄ | 20 | g |
| MgCl₂•6H₂O | 5 | g |
| CaCO₃ | 1 | g |

| | |
|---|---|
| Adjust pH with NaOH | 6.5 |

Sterilsation by autoclaving at 121° C. for 20 minutes.

### Medium 3—ISP2 Medium

In 1 L of distilled water

| | | |
|---|---|---|
| Malt extract | 10 | g |
| Yeast extract | 4 | g |
| Dextrose | 4 | g |
| Agar | 15 | g |

| | |
|---|---|
| Adjust pH with NaOH | 7.3 |

Sterilsation by autoclaving at 121° C. for 20 minutes.

### Medium 4—MAM

In 1 L of distilled water

| | | |
|---|---|---|
| Wheat starch | 10 | g |
| Corn steep solids | 2.5 | g |
| Yeast extract | 3 | g |
| CaCO₃ | 3 | g |
| Iron sulphate | 0.3 | g |
| Agar | 20 | g |

Sterilsation by autoclaving at 121° C. for 20 minutes.

### Extraction of Culture Broths for LCMS Analysis

Culture broth (1 mL) and ethyl acetate (1 mL) were mixed vigorously for 15-30 min followed by centrifugation for 10 min. 0.5 mL of the organic layer was collected, evaporated to dryness and then re-dissolved in 0.25 mL of methanol.

### LCMS Analysis Procedure

Analytical LCMS was performed using LCMS method 1 on an Agilent HP1100 HPLC system in combination with a Bruker Daltonics Esquire 3000+ electrospray mass spectrometer operating in positive and/or negative ion mode. LCMS method 1: chromatography was achieved over a Phenomenex Hyperclone column (C₁₈ BDS, 3 micron particle size, 150×4.6 mm) eluting at a flow rate of 1 mL/min using the following gradient elution process; T=0, 10% B; T=2, 10% B; T=20, 100% B; T=22, 100% B; T=22.05, 10% B; T=25, 10% B. Mobile phase A=water+0.1% formic acid; mobile phase B=acetonitrile+0.1% formic acid. UV spectra were recorded between 190 and 400 nm, with extracted chromatograms taken at 210, 254 and 276 nm. Mass spectra were recorded between 100 and 1500 amu.

### NMR Structure Elucidation Methods

NMR spectra were recorded on a Bruker Advance 500 spectrometer at 298K operating at 500 MHz and 125 MHz for ¹H and ¹³C respectively. Standard Bruker pulse sequences were used to acquire ¹H-¹H COSY, APT, HMBC and HMQC spectra. NMR spectra were referenced to the residual proton or standard carbon resonances of the solvents in which they were run.

### Assessment of Compound Purity

Purified compounds were analysed using LCMS method 2 described. LCMS method 2: chromatography was achieved over a Phenomenex HyperClone C₁₈-BDS column (4.6×150 mm, 3 micron particle size) eluting with a gradient of water+0.1% formic acid:acetonitrile+0.1% formic acid, (90:10) to (0:100), at 1 mL/min over 20 min. Purity was assessed by MS and at multiple wavelengths (210, 254 & 276 nm). All compounds were >95% pure at all wavelengths. Purity was finally confirmed by inspection of the ¹H and ¹³C NMR spectra.

### Assessment of Water Solubility

Water solubility may be tested as follows: A 10 mM stock solution of the 18,21-didesoxymacbecin analogue is prepared in 100% DMSO at room temperature. Triplicate 0.01 mL aliquots are made up to 0.5 mL with either 0.1 M PBS, pH 7.3 solution or 100% DMSO in amber vials. The resulting 0.2 mM solutions are shaken in the dark, at room temperature on an IKA® vibrax VXR shaker for 6 h, followed by transfer of the resulting solutions or suspensions into 2 mL Eppendorf tubes and centrifugation for 30 min at 13200 rpm. Aliquots of the supernatant fluid are then analysed by LCMS method 1 as described above.

### In Vitro Bioassay for Anticancer Activity

In vitro evaluation of compounds for anticancer activity in a panel of human tumour cell lines in a monolayer proliferation assay were carried out at the Oncotest Testing Facility, Institute for Experimental Oncology, Oncotest GmbH, Freiburg. The characteristics of the selected cell lines are summarised in Table 1.

TABLE 1
Test cell lines

| # | Cell line | Characteristics |
|---|---|---|
| 1 | CNXF 498NL | CNS |
| 2 | CXF HT29 | Colon |
| 3 | LXF 1121L | Lung, large cell ca |
| 4 | MCF-7 | Breast, NCI standard |
| 5 | MEXF 394NL | Melanoma |
| 6 | DU145 | Prostate - PTEN positive |

The Oncotest cell lines were established from human tumor xenografts as described by Roth et al., (1999). The origin of the donor xenografts was described by Fiebig et al., (1999). Other cell lines were either obtained from the NCI (DU145, MCF-7) or purchased from DSMZ, Braunschweig, Germany.

All cell lines, unless otherwise specified, were grown at 37° C. in a humidified atmosphere (95% air, 5% CO₂) in a ‘ready-mix’ medium containing RPMI 1640 medium, 10% fetal calf serum, and 0.1 mg/mL gentamicin (PAA, Cölbe, Germany).

A modified propidium iodide assay was used to assess the effects of the test compound(s) on the growth of human tumour cell lines (Dengler et al., (1995)).

Briefly, cells were harvested from exponential phase cultures by trypsinization, counted and plated in 96 well flat-bottomed microtitre plates at a cell density dependent on the cell line (5-10.000 viable cells/well). After 24 h recovery to allow the cells to resume exponential growth, 0.010 mL of culture medium (6 control wells per plate) or culture medium containing the 18,21-didesoxymacbecin analogue was added to the wells. Each concentration was plated in triplicate. Compounds were applied in five concentrations (100; 10; 1; 0.1 and 0.01 μg/ml). Following 4 days of continuous exposure, cell culture medium with or without test compound was replaced by 0.2 mL of an aqueous propidium iodide (PI) solution (7 mg/L). To measure the proportion of living cells, cells may be permeabilized by freezing the plates. After thawing the plates, fluorescence was measured using the Cytofluor 4000 microplate reader (excitation 530 nm, emission 620 nm), giving a direct relationship to the total number of viable cells. Growth inhibition may be expressed as treated/control×100 (% T/C). This can be plotted as a graph of % T/C against concentration of test compound applied, which can then be used to calculate the concentration necessary to inhibit cell growth by 70% (IC₇₀).

### Example 1

### Sequencing of the Macbecin PKS Gene Cluster

Genomic DNA was isolated from Actinosynnema pretiosum (ATCC 31280) and Actinosynnema mirum (DSM 43827, ATCC 29888) using standard protocols described in Kieser et al., (2000). DNA sequencing was carried out by the sequencing facility of the Biochemistry Department, University of Cambridge, Tennis Court Road, Cambridge CB21QW using standard procedures.

Primers BIOSG104 5′-GGTCTAGAGGTCAGTGCCCCCGCGTACCGTCGT-3′ (SEQ ID NO: 1) AND BIOSG105 5′-GGCATATGCTTGTGCTCGGGCTCAAC-3′ (SEQ ID NO: 2) were employed to amplify the carbamoyltransferase-encoding gene gdmN from the geldanamycin biosynthetic gene cluster of Streptomyces hygroscopicus NRRL 3602 (Accession number of sequence: AY179507) using standard techniques. Southern blot experiments were carried out using the DIG Reagents and Kits for Non-Radioactive Nucleic Acid Labelling and Detection according to the manufacturers' instructions (Roche). The DIG-labelled gdmN DNA fragment was used as a heterologous probe. Using the gdmN generated probe and genomic DNA isolated from A. pretiosum 2112 an approximately 8 kb EcoRI fragment was identified in Southern blot analysis. The fragment was cloned into Litmus 28 applying standard procedures and transformants were identified by colony hybridization. The clone p3 was isolated and the approximately 7.7 kb insert was sequenced. DNA isolated from clone p3 was digested with EcoRI and EcoRI/SacI and the bands at around 7.7 kb and at about 1.2 kb were isolated, respectively. Labelling reactions were carried out according to the manufacturers' protocols. Cosmid libraries of the two strains named above were created using the vector SuperCos 1 and the Gigapack III XL packaging kit (Stratagene) according to the manufacturers' instructions. These two libraries were screened using standard protocols and as a probe, the DIG-labelled fragments of the 7.7 kb EcoRI fragment derived from clone p3 were used. Cosmid 52 was identified from the cosmid library of A. pretiosum and submitted for sequencing to the sequencing facility of the Biochemistry Department of the University of Cambridge. Similarly, cosmid 43 and cosmid 46 were identified from the cosmid library of A. mirum. All three cosmids contain the 7.7 kb EcoRI fragment as shown by Southern Blot analysis.

An around 0.7 kbp fragment of the PKS region of cosmid 43 was amplified using primers BIOSG124 5′-CCCGCCCGCGCGAGCGGCGCGTGGCCGCCCGAGGGC-3′ (SEQ ID NO: 3) and BIOSG125 5′-GCGTCCTCGCGCAGCCACGCCACCAGCAGCTCCAGC-3′ (SEQ ID NO: 4) applying standard protocols, cloned and used as a probe for screening the A. pretiosum cosmid library for overlapping clones. The sequence information of cosmid 52 was also used to create probes derived from DNA fragments amplified by primers BIOSG130 5′-CCAACCCCGCCGCGTCCCCGGCCGCGCCGAACACG-3′ (SEQ ID NO: 5) and BIOSG131 5′-GTCGTCGGCTACGGGCCGGTGGGGCAGCTGCTGT-5′ (SEQ ID NO: 6) as well as BIOSG132 5′-GTCGGTGGACTGCCCTGCGCCTGATCGCCCTGCGC-3′ (SEQ ID NO: 7) and BIOSG133 5′-GGCCGGTGGTGCTGCCCGAGGACGGGGAGCTGCGG-3′ (SEQ ID NO: 8) which were used for screening the cosmid library of A. pretiosum. Cosmids 311 and 352 were isolated and cosmid 352 was sent for sequencing. Cosmid 352 contains an overlap of approximately 2.7 kb with cosmid 52. To screen for further cosmids, an approximately 0.6 kb PCR fragment was amplified using primers BIOSG136 5′-CACCGCTCGCGGGGGTGGCGCGGCGCACGACGTGG CTGC-3′ (SEQ ID NO: 9) and BIOSG137 5′-CCTCCTCGGACAGCGCGATCAGCGCCGCGC ACAGCGAG-3′ (SEQ ID NO: 10) and cosmid 311 as template applying standard protocols. The cosmid library of A. pretiosum was screened and cosmid 410 was isolated. It overlaps approximately 17 kb with cosmid 352 and was sent for sequencing. The sequence of the three overlapping cosmids (cosmid 52, cosmid 352 and cosmid 410) was assembled. The sequenced region spans about 100 kbp and 23 open reading frames were identified potentially constituting the macbecin biosynthetic gene cluster. The location of each of the open reading frames within SEQ ID NO: 11 is shown in Table 3

TABLE 2
Summary of the cosmids

| Cosmid | Strain |
|---|---|
| Cosmid 43 | Actinosynnema mirum ATCC 29888 |
| Cosmid 46 | Actinosynnema mirum ATCC 29888 |
| Cosmid 52 | Actinosynnema pretiosum ATCC 31280 |
| Cosmid 311 | Actinosynnema pretiosum ATCC 31280 |
| Cosmid 352 | Actinosynnema pretiosum ATCC 31280 |
| Cosmid 410 | Actinosynnema pretiosum ATCC 31280 |

TABLE 3
location of each of the open reading frames for the post-PKS genes
and the starter unit biosynthesis genes

| Nucleotide position in | | Function of the encoded |
|---|---|---|
| SEQ ID NO: 11 | Gene Name | protein |
| 14925-17909* | mbcRII | transcriptional regulator |
| 18025-19074c | mbcO | aminohydroquinate synthase |
| 19263-20066c* | mbc? | unknown, AHBA biosynthesis |
| 20330-40657 | mbcAI | PKS |
| 40654-50859 | mbcAII | PKS |
| 50867-62491* | mbcAIII | PKS |
| 62500-63276* | mbcF | amide synthase |
| 63281-64852* | mbcM | C21 monooxygenase |
| 64899-65696c* | PH | phosphatase |
| 65693-66853c* | OX | oxidoreductase |
| 66891-68057c* | Ahs | AHBA synthase |
| 68301-68732* | Adh | ADHQ dehydratase |
| 68690-69661c* | AHk | AHBA kinase |
| 70185-72194c* | mbcN | carbamoyltransferase |
| 72248-73339c | mbcH | methoxymalonyl ACP pathway |
| 73336-74493c | mbcI | methoxymalonyl ACP pathway |
| 74490-74765c | mbcJ | methoxymalonyl ACP pathway |
| 74762-75628c* | mbcK | methoxymalonyl ACP pathway |
| 75881-76537 | mbcG | methoxymalonyl ACP pathway |
| 76534-77802* | mbcP | C4,5 monooxygenase |
| 77831-79054* | mbcP450 | P450 |
| 79119-79934* | mbcMT1 | O-methyltransferase |
| 79931-80716* | mbcMT2 | O-methyltransferase |

[Note 1: c indicates that the gene is encoded by the complement DNA strand; Note 2: it is sometimes the case that more than one potential candidate start codon can been identified. One skilled in the art will recognise this and be able to identify alternative possible start codons. We have indicated those genes which have more than one possible start codon with a ^{‘}*^{’}symbol. Throughout we have indicated what we believe to be the start codon, however, a person of skill in the art will appreciate that it may be possible to generate active protein using an alternative start codon.]

### Example 2

### Generation of Strain BIOT-3806: an Actinosynnema pretiosum Strain in which the gdmM Homologue mcbM has been Interrupted by Insertion of a Plasmid

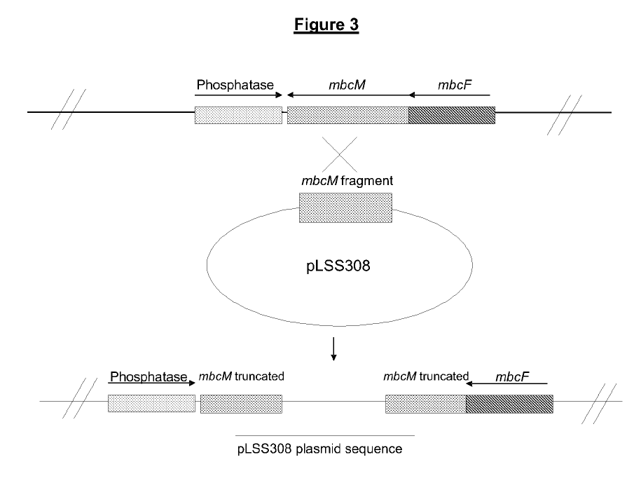

A summary of the construction of pLSS308 is shown in FIG. 3.

2.1. Construction of Plasmid pLSS308

The DNA sequences of the gdmM gene from the geldanamycin biosynthetic gene cluster of Streptomyces hygroscopicus strain NRRL 3602 (AY179507) and orf19 from the rifamycin biosynthetic gene cluster of Amycolatopsis mediterranei (AF040570 AF040571) were aligned using Vector NTI sequence alignment program. This alignment identified regions of homology that were suitable for the design of degenerate oligos that were used to amplify a fragment of the homologous gene from Actinosynnema mirum (BIOT-3134; DSM43827; ATCC29888). The degenerate oligos are:

FPLS1:

(SEQ ID NO: 12)

5′: ccscgggcgnycngsttcgacngygag 3′;
FPLS3:

(SEQ ID NO: 13)

5′: cgtcncggannccggagcacatgccctg 3′;

where N=G, A, T or C; Y=C or T; S=G or C

The template for PCR amplification was Actinosynnema mirum cosmid 43. The generation of cosmid 43 is described in Example 1 above.

Oligos FPLS1 and FPLS3 were used to amplify the internal fragment of a gdmM homologue from Actinosynnema mirum in a standard PCR reaction using cosmid 43 as the template and Taq DNA polymerase. The resultant 793 bp PCR product was cloned into pUC19 that had been linearised with SmaI, resulting in plasmid pLSS301. It was postulated that the amplified sequence is from the mcbM gene of the macbecin cluster of A. mirum. Plasmid pLSS301 was digested with EcoRI/HindIII and the fragment cloned into plasmid pKC1132 (Bierman et al., 1992) that had been digested with EcoRI/HindIII. The resultant plasmid, designated pLSS308, is apramycin resistant and contains an internal fragment of the A. mirum mbcM gene.

2.2 Transformation of Actinosynnema pretiosum Subsp. pretiosum

Escherichia coli ET12567, harbouring the plasmid pUZ8002 was transformed with pLSS308 by electroporation to generate the E. coli donor strain for conjugation. This strain was used to transform Actinosynnema pretiosum subsp. pretiosum by vegetative conjugation (Matsushima et al., 1994). Exconjugants were plated on Medium 4 and incubated at 28° C. Plates were overlayed after 24 h with 50 mg/L apramycin and 25 mg/L nalidixic acid. As pLSS308 is unable to replicate in Actinosynnema pretiosum subsp. pretiosum, any apramycin resistant colonies were anticipated to be transformants that contained plasmid integrated into the mbcM gene of the chromosome by homologous recombination via the plasmid borne mcbM internal fragment (FIG. 3). This results in two truncated copies of the mbcM gene on the chromosome. Transformants were confirmed by PCR analysis and the amplified fragment was sequenced.

Colonies were patched onto Medium 4 (with 50 mg/L apramycin and 25 mg/L nalidixic acid). A 6 mm circular plug from each patch was used to inoculate individual 50 mL falcon tubes containing 10 mL seed medium (variant of Medium 1-2% glucose, 3% soluble starch, 0.5% corn steep solids, 1% soybean flour, 0.5% peptone, 0.3% sodium chloride, 0.5% calcium carbonate) plus 50 mg/L apramycin. These seed cultures were incubated for 2 days at 28° C., 200 rpm with a 5 cm throw. These were then used to inoculate (5% v/v) fermentation medium (Medium 2) and were grown at 28° C. for 24 hours and then at 26° C. for a further 5 days. Metabolites were extracted from these according to the standard protocol described above. Samples were assessed for production of macbecin analogues by HPLC using the standard protocol described above.

The productive isolate selected was designated BIOT-3806.

2.3 Identification of Compounds from BIOT-3806

Samples were analysed as described in General Methods using LCMS method 1.

TABLE 4
compounds identified by LCMS

| | Retention | | | |
|---|---|---|---|---|
| Compound | time (min) | [M + Na]⁺ | [M − H]⁻ | Mass |
| 14 | 11.4 | 525.2 | 501.2 | 502 |
| 15 | 9.7 | 541.1 | 517.1 | 518 |
| A | 8.6 | 506.1 | 482.1 | 483 |
| B | 9.3 | 539.2 | 515.1 | 516 |
| C | 10.9 | 543.1 | 519.2 | 520 |

### Example 3

### Generation of BIOT-3870: an Actinosynnema pretiosum Strain in which the gdmM Homologue mbcM has an In-Frame Deletion

3.1 Cloning of DNA Homologous to the Downstream Flanking Region of mbcM.

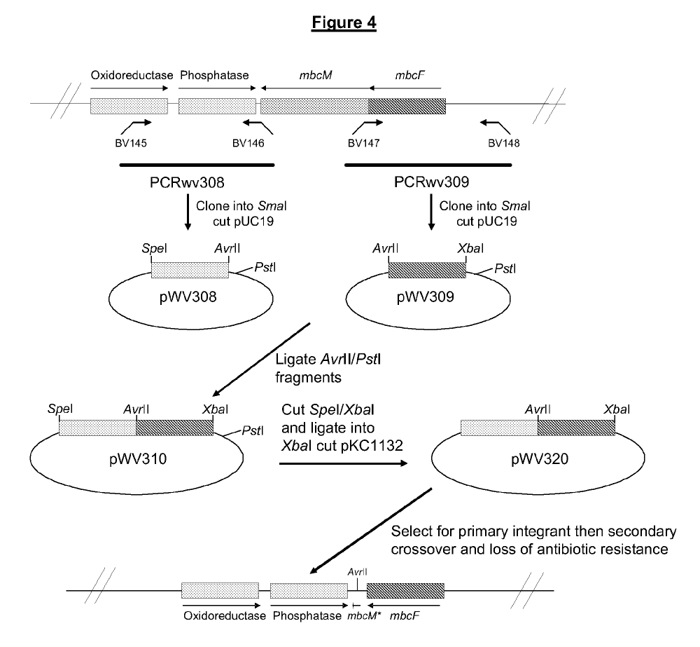

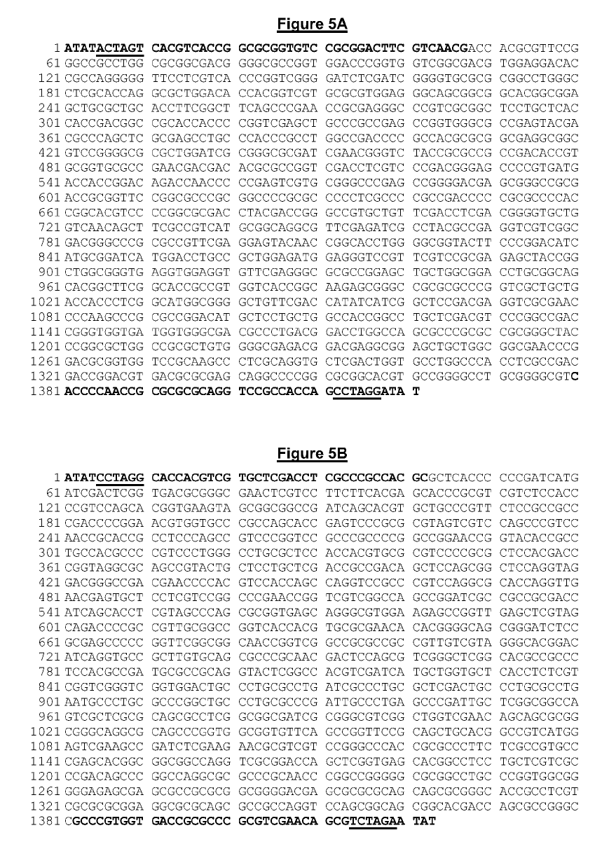

Oligos BV145 (SEQ ID NO: 14) and BV146 (SEQ ID NO: 15) were used to amplify a 1421 bp region of DNA from Actinosynnema pretiosum (ATCC 31280) in a standard PCR reaction using cosmid 52 (from example 1) as the template and Pfu DNA polymerase. A 5′ extension was designed in each oligo to introduce restriction sites to aid cloning of the amplified fragment (FIG. 4). The amplified PCR product (PCRwv308, SEQ ID NO: 16, FIG. 5A) encoded 33 bp of the 3′ end of mbcM and a further 1368 bp of downstream homology. This 1421 bp fragment was cloned into pUC19 that had been linearised with SmaI, resulting in plasmid pWV308.

BV145

(SEQ ID NO: 14)

ATATACTAGTCACGTCACCGGCGCGGTGTCCGCGGACTTCGTCAACG
SpeI
BV146

(SEQ ID NO: 15)

ATATCCTAGGCTGGTGGCGGACCTGCGCGCGCGGTTGGGGTG
AvrII

3.2 Cloning of DNA Homologous to the Upstream Flanking Region of mbcM.

Oligos BV147 (SEQ ID NO: 17) and BV148 (SEQ ID NO: 18) were used to amplify a 1423 bp region of DNA from Actinosynnema pretiosum (ATCC 31280) in a standard PCR reaction using cosmid 52 (from example 1) as the template and Pfu DNA polymerase. A 5′ extension was designed in each oligo to introduce restriction sites to aid cloning of the amplified fragment (FIG. 4). The amplified PCR product (PCRwv309, SEQ ID NO: 19, FIG. 5B) encoded 30 bp of the 5′ end of mbcM and a further 1373 bp of upstream homology. This 1423 bp fragment was cloned into pUC19 that had been linearised with SmaI, resulting in plasmid pWV309.

BV147

(SEQ ID NO: 17)

ATATCCTAGGCACCACGTCGTGCTCGACCTCGCCCGCCACGC
AvrII
BV148

(SEQ ID NO: 18)

ATATTCTAGACGCTGTTCGACGCGGGCGCGGTCACCACGGGC
XbaI

The products PCRwv308 and PCRwv309 were cloned into pUC19 in the same orientation to utilise the PstI site in the pUC19 polylinker for the next cloning step.

The 1443 bp AvrII/PstI fragment from pWV309 was cloned into the 4073 bp AvrII/PstI fragment of pWV308 to make pWV310. pWV310 therefore contained a SpeI/XbaI fragment encoding DNA homologous to the flanking regions of mbcM fused at an AvrII site. This 2816 bp SpeI/XbaI fragment was cloned into pKC1132 (Bierman et al., 1992) that had been linearised with SpeI to create pWV320.

3.3 Transformation of Actinosynnema pretiosum Subsp. pretiosum

Escherichia coli ET12567, harbouring the plasmid pUZ8002 was transformed with pWV320 by electroporation to generate the E. coli donor strain for conjugation. This strain was used to transform Actinosynnema pretiosum subsp. pretiosum by vegetative conjugation (Matsushima et al., 1994). Exconjugants were plated on Medium 4 and incubated at 28° C. Plates were overlayed after 24 h with 50 mg/L apramycin and 25 mg/L nalidixic acid. As pWV320 is unable to replicate in Actinosynnema pretiosum subsp. pretiosum, apramycin resistant colonies were anticipated to be transformants that contained plasmid pWV320 integrated into the chromosome by homologous recombination via one of the plasmid borne mbcM flanking regions of homology.

Genomic DNA was isolated from six exconjugants and was digested and analysed by Southern blot. The blot showed that in four out of the six isolates integration had occurred in the upstream region of homology and in two of the six isolates homologous integration had occurred in the downstream region. One strain resulting from homologous integration in the upstream region (designated BIOT-3831) was chosen for screening for secondary crosses. One strain resulting from homologous integration in the downstream region (BIOT-3832) was also chosen for screening for secondary crosses.

### 3.4 Screening for Secondary Crosses

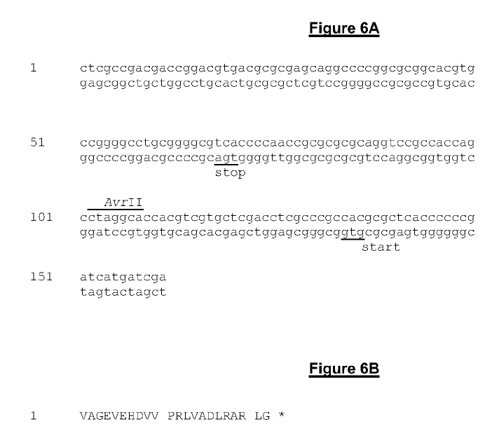

Strains were patched onto medium 4 (supplemented with 50 mg/L apramycin) and grown at 28° C. for four days. A 1 cm² section of each patch was used to inoculate 7 mL modified ISP2 (0.4% yeast extract, 1% malt extract, 0.4% dextrose in 1 L distilled water) without antibiotic in a 50 mL falcon tube. Cultures were grown for 2-3 days then subcultured on (5% inoculum) into another 7 mL modified ISP2 (see above) in a 50 mL falcon tube. After 4-5 generations of subculturing the cultures were sonicated, serially diluted, plated on Medium 4 and incubated at 28° C. for four days. Single colonies were then patched in duplicate onto Medium 4 containing apramycin and onto Medium 4 containing no antibiotic and the plates were incubated at 28° C. for four days. Patches that grew on the no antibiotic plate but did not grow on the apramycin plate were re-patched onto +/−apramycin plates to confirm that they had lost the antibiotic marker. The mutant strain encodes an mbcM protein with an in-frame deletion of 502 amino acids (FIG. 6A, SEQ ID NOs: 20 and 21; FIG. 6B shows the encoded protein sequence, SEQ ID NO: 22).

mbcM deletion mutants were patched onto Medium 4 and grown at 28° C. for four days. A 6 mm circular plug from each patch was used to inoculate individual 50 mL falcon tubes containing 10 mL seed medium (adapted from medium 1-2% glucose, 3% soluble starch, 0.5% corn steep solids, 1% soybean flour, 0.5% peptone, 0.3% sodium chloride, 0.5% calcium carbonate). These seed cultures were incubated for 2 days at 28° C., 200 rpm with a 2 inch throw. These were then used to inoculate (0.5 mL into 10 mL) production medium (medium 2-5% glycerol, 1% corn steep solids, 2% yeast extract, 2% potassium dihydrogen phosphate, 0.5% magnesium chloride, 0.1% calcium carbonate) and were grown at 28° C. for 24 hours and then at 26° C. for a further 5 days. Secondary metabolites were extracted and analysed by LCMS for production of macbecin analogues as described in General Methods.

3.5 Identification of 14 and 15 from BIOT-3872

Extracts of the fermentation described in Example 3.4 were generated and assayed by LCMS as described in General Methods using LCMS method 1. No macbecin was observed and two new major components were observed. The compounds displayed the physiochemical characteristics shown in Table 5 below:

TABLE 5
compounds identified by LCMS

| | Retention | | | |
|---|---|---|---|---|
| | time | | | |
| Compound | (min) | [M + Na]⁺ | [M − H]⁻ | Mass |
| 14 | 11.5 | 525.2 | 501.2 | 502 |
| 15 | 9.9 | 541.1 | 517.1 | 518 |

Compounds 14 and 15 were shown to be identical to the mabcein analogues 7-O-carbamoylpre-macbecin and 7-O-carbamoyl-15-hydroxypre-macbecin that have been reported previously

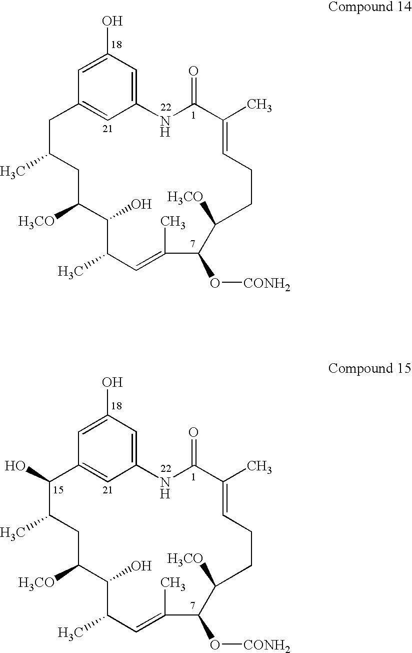

Note that removal of the function of MbcM either by integration into mbcM (Example 2) or deletion of the mbcM gene produces the same compounds; 14 and 15. Analysis of the relationship between the observed structures and the biosynthetic pathway indicates that a number of enzymes are not functioning in addition to MbcM. In the case of compound 15 these are MbcP, MbcMT1 and MbcMT2 and in the case of compound 14 function of MbcP450 is also not observed. As described above there can be a number of reasons why these proteins may not be functional in this system, for example compounds 14 and 15 represent novel structures for these enzymes and they may poor substrates or not substrates at all.

### 3.6 Selection of Individual Colonies by Generating Protoplasts of BIOT-3872

Protoplasts were generated from BIOT-3872 using a method adapted from Weber and Losick 1988 with the following media alterations; Actinosynnema pretiosum cultures were grown on ISP2 plates (medium 3) for 3 days at 28° C. and a 5 mm² scraping used to inoculate 40 ml of ISP2 broth supplemented with 2 ml of sterile 10% (w/v) glycine in water. Protplasts were generated as described in Weber and Losick 1988 and then regenerated on R2 plates (R2 recipe—Sucrose 103 g, K₂SO₄ 0.25 g, MgCl₂.6H₂O 10.12 g, Glucose 10 g, Difco Casaminoacids 0.1 g, Difco Bacto agar 22 g, distilled water to 800 mL, the mixture was sterilised by autoclaving at 121° C. for 20 minutes. After autoclaving the following autoclaved solutions were added; 0.5% KH₂PO₄ 10 ml, 3.68% CaCl₂.2H₂O 80 mL, 20% L-proline 15 mL, 5.73% TES buffer (pH7.2) 100 mL, Trace element solution (ZnCl₂ 40 mg, FeCl₃.6H₂O 200 mg, CuCl₂.2H₂O 10 mg, MnCl₂.4H₂O 10 mg, Na₂B₄O₇.10H₂O 10 mg, (NH₄)₆Mo₇O₂₄.4H₂O 10 mg, distilled water to 1 litre) 2 mL, NaOH (1 N) (unsterilised) 5 mL).

80 individual colonies were patched onto MAM plates (medium 4) and analysed for production of macbecin analogues as described above. The majority of protoplast generated patches produced at similar low levels to the parental strain. 15 out of the 80 samples tested produced significantly more 14 and 15 than the parental strain. The best producing strain, BIOT-3870 (also named WV4a-33) was observed to produce 14 and 15 at significantly higher levels than the parent strain and was selected for use in future experiments.

### Example 4

### Feeding to WV4a-33 to Generate 4,5-dihydro-11-O-desmethyl-15-desmethoxy-18,21-didesoxymacbecin

4.1 Biotransformation of 3-Amino-Benzoic Acid with WV4a-33 (BIOT-3870)

WV4a-33 was patched onto MAM plates (medium 4) and grown at 28° C. for three days. A 6 mm circular plug was used to inoculate individual 50 mL falcon tubes containing 10 mL seed medium (adapted from medium 1-2% glucose, 3% soluble starch, 0.5% corn steep solids, 1% soybean flour, 0.5% peptone, 0.3% sodium chloride, 0.5% calcium carbonate). These seed cultures were incubated for 65 hours at 28° C., 200 rpm with a 2 inch throw. These were then used to inoculate (1 mL into 10 mL) modified production medium (adapted from medium 2-5% glycerol, 1% corn steep solids, 2% yeast extract, 2% potassium dihydrogen phosphate, 0.5% magnesium chloride, 0.1% calcium carbonate media is left to sediment for 2-60 days and the top layer is taken as the production medium) and were grown at 26° C. for 24 hours. 0.1 mL of a 200 mM feed stock solution (3-aminobenzoic acid dissolved in methanol) was added to each falcon tube to give a final feed concentration of 2 mM. Tubes were incubated for a further 6 days at 26° C. In parallel, seed cultures were used to inoculate medium 2. Analysis of these cultures (see below) showed that identical compounds were produced in both types of production media but higher titres were observed when using the modified media.

4.2 Identification of 4,5-dihydro-11-O-desmethyl-15-desmethoxy-18,21-didesoxymacbecin from Cultures of WV4a-33 Fed with 3-aminobenzoic Acid

Extracts of the fermentation described in example 2.7 were generated and assayed by LCMS as described in General Methods. The compounds 14 and 15 were produced as expected. In addition a new compound 16 was clearly observed which could not be seen in extracts of any fermentations that were not fed 3-aminobenzoic acid. 16 eluted later than either 14 or 15 and had the physiochemical characteristics described in Table 6 below.

Based on the available data 16 was identified as 4,5-dihydro-11-O-desmethyl-15-desmethoxy-18,21-didesoxymacbecin

TABLE 6
compounds identified by LCMS

| | Retention | | | |
|---|---|---|---|---|
| | time | | | |
| Compound | (min) | [M + Na]⁺ | [M − H]⁻ | Mass |
| 14 | 11.5 | 525.2 | 501.2 | 502 |
| 15 | 9.9 | 541.1 | 517.1 | 518 |
| 16 | 12.9 | 509.3 | 485.2 | 486 |

### Example 5

### Production and Isolation of 4,5-dihydro-11-O-desmethyl-15-desmethoxy-18,21-didesoxymacbecin (Alternative Method)

5.1 Fermentation of 4,5-dihydro-11-O-desmethyl-15-desmethoxy-18,21-didesoxymacbecin from Cultures of WV4a-33 Fed with 3-aminobenzoic Acid

WV4a-33 was patched onto MAM plates (medium 4) and grown at 28° C. for three days. Two 6 mm circular plugs were used to inoculate 250 ml conical shake flasks containing 30 mL seed medium (adapted from medium 1-2% glucose, 3% soluble starch, 0.5% corn steep solids, 1% soybean flour, 0.5% peptone, 0.3% sodium chloride, 0.5% calcium carbonate). Six flasks were inoculated. These seed cultures were incubated for 65 hours at 28° C., 200 rpm with a 1 inch throw. These were then used to inoculate (1 mL into 10 mL) 170 falcon tubes each containing 10 ml of modified production medium (adapted from medium 2-5% glycerol, 1% corn steep solids, 2% yeast extract, 2% potassium dihydrogen phosphate, 0.5% magnesium chloride, 0.1% calcium carbonate media is left to sediment for 2-60 days and the top layer is taken as the production medium) and were grown at 26° C. for 24 hours. 0.1 mL of a 200 mM feed stock solution (3-aminobenzoic acid dissolved in methanol) was added to each falcon tube to give a final feed concentration of 2 mM. Tubes were incubated for a further 6 days at 26° C. The cultures were pooled (approximately 1.4 l) and the falcon tubes were washed (each with 7 ml of water). The washing liquid was pooled (approximately 1.4 l). The pooled cultures and washing liquids were used for isolation of 4,5-dihydro-11-O-desmethyl-15-desmethoxy-18,21-didesoxymacbecin (approximately 3 L in total). In parallel, the seed cultures were used to inoculate 30 ml of modified production medium (3 ml) followed by the same incubation and feeding regime as described above (final feed concentration of 2 mM). The flasks were incubated in a 2 inch throw shaker. Production levels were estimated by LCMS as being approximately between 50% and 90% of those measured for the falcon tube production cultures.

5.2 Isolation and Characterisation of 4,5-dihydro-11-O-desmethyl-15-desmethoxy-18,21-didesoxymacbecin

The fermentation broth (3 L) was extracted two times with an equal volume of ethyl acetate (EtOAc). The organic extracts were combined and the solvent removed in vacuo at 40° C. to yield 1.2 g of an oily residue. This residue was then chromatographed over Silica gel 60 column (30×2.5 cm) with a stepped gradient from 100% CHCl₃ to CHCl₃:MeOH (97:3) and collecting fractions of approx. 250 mL. The fractions were monitored by analytical HPLC. Fractions containing 16 were combined and solvent removed in vacuo at 40° C. to yield 435 mg of semi-pure 16. This semi-pure material was further purified by reversed-phase HPLC over a Phenomenex-Luna C₁₈-BDS column (21.2×250 mm, 5 micron particle size) eluting with a gradient of water:acetonitrile, (77:23) to (20:80), over 25 min at a flow rate of 21 ml/min. 16 eluted at 17 min and the relevant fractions were combined, the solvent removed at reduced pressure to yield 16 as a white powder (125 mg).

The purity of 16 was confirmed by LCMS using method 1 as described in General Methods. LCMS: 16, R=12.9 min ([M+Na]⁺, m/z=509.4; [M−H]⁻, m/z=485.5.

Proton NMR data collected at 400 MHz was consistent with the structure shown.

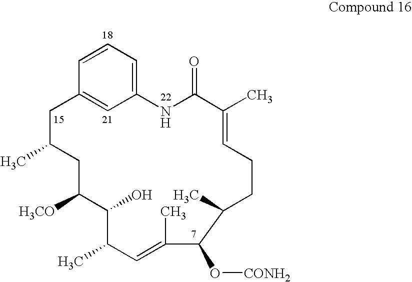

### Example 6

### Generation of 4,5-dihydro-11-O-desmethyl-15-desmethoxy-17-fluoro-18,21-didesoxymacbecin by Feeding 5-amino-2-fluorobenzoic acid to BIOT-3870

6.1 Biotransformation of 5-amino-2-fluorobenzoic acid with BIOT-3870

BIOT-3870 was patched onto MAM plates (medium 4) and grown at 28° C. for three days. A 6 mm circular plug was used to inoculate individual 50 mL falcon tubes containing 10 mL seed medium (adapted from medium 1-2% glucose, 3% soluble starch, 0.5% corn steep solids, 1% soybean flour, 0.5% peptone, 0.3% sodium chloride, 0.5% calcium carbonate). These seed cultures were incubated for 65 hours at 28° C., 200 rpm with a 2 inch throw. These were then used to inoculate (1 mL into 10 mL) modified production medium (adapted from medium 2-5% glycerol, 1% corn steep solids, 2% yeast extract, 2% potassium dihydrogen phosphate, 0.5% magnesium chloride, 0.1% calcium carbonate media is left to sediment for 2-60 days and the top layer is taken as the production medium) and were grown at 26° C. for 24 hours. 0.1 mL of a 200 mM feed stock solution (5-amino-2-fluorobenzoic acid dissolved in methanol) was added to each falcon tube to give a final feed concentration of 2 mM. Tubes were incubated for a further 6 days at 26° C.

6.2 Identification of 4,5-dihydro-11-O-desmethyl-15-desmethoxy-17-fluoro-18,21-didesoxymacbecin, 17

Analysis was performed as described in General Methods using LCMS method 1. In addition to 14 and 15a new compound was observed with LCMS charateristics described in Table 7. These data were consistent with the title compound.

TABLE 7

| | Retention time | | | |
|---|---|---|---|---|
| Compound | (min) | [M + Na]⁺, m/z | [M − H]⁻, m/z | Mass |
| 14 | 11.5 | 525.2 | 501.2 | 502 |
| 15 | 9.9 | 541.1 | 517.1 | 518 |
| 17 | 13.3 | 527.3 | 503.3 | 504 |

6.3 Production and extraction of 4,5-dihydro-11-O-desmethyl-15-desmethoxy-17-fluoro-18,21-didesoxymacbecin, 17

BIOT-3870 was patched onto MAM plates (medium 4) and grown at 28° C. for three days. Two 6 mm circular plugs were used to inoculate 250 ml conical shake flasks containing 30 mL seed medium (adapted from medium 1-2% glucose, 3% soluble starch, 0.5% corn steep solids, 1% soybean flour, 0.5% peptone, 0.3% sodium chloride, 0.5% calcium carbonate). Six flasks were inoculated. These seed cultures were incubated for 65 hours at 28° C., 200 rpm with a 1 inch throw. These were then used to inoculate (1 mL into 10 mL) 170 falcon tubes each containing 10 mL of modified production medium (adapted from medium 2-5% glycerol, 1% corn steep solids, 2% yeast extract, 2% potassium dihydrogen phosphate, 0.5% magnesium chloride, 0.1% calcium carbonate media is left to sediment for 2-60 days and the top layer is taken as the production medium) and were grown at 26° C. for 24 hours. 0.1 mL of a 200 mM feed stock solution (5-amino-2-fluorobenzoic acid dissolved in methanol) was added to each falcon tube to give a final feed concentration of 2 mM. Tubes were incubated for a further 6 days at 26° C. The cultures were pooled (approximately 1.4 L) and the falcon tubes were washed (each with 7 mL of water). The washing liquid was pooled (approximately 1.4 L). The pooled cultures and washing liquids were used for isolation of 4,5-dihydro-11-O-desmethyl-15-desmethoxy-17-fluoro-18,21-didesoxymacbecin see below.

6.4 Purification and Characterisation of 4,5-dihydro-11-O-desmethyl-15-desmethoxy-17-fluoro-18,21-didesoxymacbecin, 17

The fermentation broth (−3 L) was extracted two times with an equal volume of ethyl acetate (EtOAc). The organic extracts were combined and the solvent removed in vacuo at 40° C. to yield 3.0 g of an oily residue. This residue was then chromatographed over Silica gel 60 column eluting with 2% methanol in CHCl₃ and collecting fractions of approx. 250 mL. The fractions were monitored by analytical HPLC. Fractions containing were combined and solvent removed in vacuo at 40° C. This semi-pure material was further purified by reversed-phase HPLC over a Phenomenex-Luna C₁₈-BDS column (21.2×250 mm, 5 micron particle size) eluting with a gradient of water:acetonitrile, (77:23) to (20:80), over 25 min at a flow rate of 21 mL/min. 17 eluted at 18 min and the relevant fractions were combined and the solvent removed at reduced pressure to yield as a white powder (54 mg). NMR data acquired in d₆-acetone were entirely consistent with the reported structure.

The purity of 17 was confirmed as described in General Methods using LCMS method 2. Measurements were taken at multiple wavelengths and using MS analysis in both positive and negative modes. LCMS: 17, RT=11.3 min ([M−H]⁻, m/z=503.3; [M+Na]⁺, m/z=527.3; [2M+Na]⁺, m/z=1032.0).

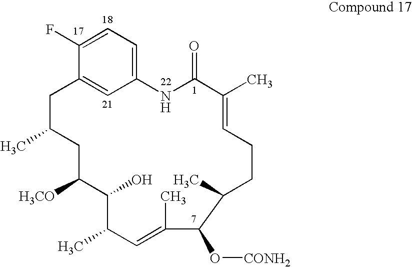

### Example 7

### Generation of 4,5-dihydro-11-O-desmethyl-15-desmethoxy-18-fluoro-18,21-didesoxymacbecin by feeding 5-amino-3-fluorobenzoic acid to BIOT-3870

7.1 Biotransformation of 5-amino-3-fluorobenzoic Acid with BIOT-3870

BIOT-3870 was patched onto MAM plates (medium 4) and grown at 28° C. for three days. A 6 mm circular plug was used to inoculate individual 50 mL falcon tubes containing 10 mL seed medium (adapted from medium 1-2% glucose, 3% soluble starch, 0.5% corn steep solids, 1% soybean flour, 0.5% peptone, 0.3% sodium chloride, 0.5% calcium carbonate). These seed cultures were incubated for 65 hours at 28° C., 200 rpm with a 2 inch throw. These were then used to inoculate (1 mL into 10 mL) modified production medium (adapted from medium 2-5% glycerol, 1% corn steep solids, 2% yeast extract, 2% potassium dihydrogen phosphate, 0.5% magnesium chloride, 0.1% calcium carbonate media is left to sediment for 2-60 days and the top layer is taken as the production medium) and were grown at 26° C. for 24 hours. 0.1 mL of a 200 mM feed stock solution (5-amino-3-fluorobenzoic acid dissolved in methanol) was added to each falcon tube to give a final feed concentration of 2 mM. Tubes were incubated for a further 6 days at 26° C.

7.2 Identification of 4,5-dihydro-11-O-desmethyl-15-desmethoxy-18-fluoro-18,21-didesoxymacbecin, 18

Analysis was performed as described in General Methods using LCMS method 1. In addition to 14 and 15 two new compounds were observed with LCMS charateristics described in Table 8. These data were consistent with the title compound, 18 and its C15-hydroxylated analogue, 19

TABLE 8

| | Retention time | | | |
|---|---|---|---|---|
| Compound | (min) | [M + Na]⁺, m/z | [M − H]⁻, m/z | Mass |
| 14 | 11.5 | 525.2 | 501.2 | 502 |
| 15 | 9.9 | 541.1 | 517.1 | 518 |
| 18 | 14.1 | 527.2 | 503.1 | 504 |
| 19 | 11.5 | 543.3 | 519.3 | 520 |

7.3 Production and Extraction of 4,5-dihydro-11-O-desmethyl-15-desmethoxy-18-fluoro-18,21-didesoxymacbecin, 18

BIOT-3870 was patched onto MAM plates (medium 4) and grown at 28° C. for three days. Two 6 mm circular plugs were used to inoculate 250 ml conical shake flasks containing 30 mL seed medium (adapted from medium 1-2% glucose, 3% soluble starch, 0.5% corn steep solids, 1% soybean flour, 0.5% peptone, 0.3% sodium chloride, 0.5% calcium carbonate). Six flasks were inoculated. These seed cultures were incubated for 65 hours at 28° C., 200 rpm with a 1 inch throw. These were then used to inoculate (1 mL into 10 mL) 170 falcon tubes each containing 10 mL of modified production medium (adapted from medium 2-5% glycerol, 1% corn steep solids, 2% yeast extract, 2% potassium dihydrogen phosphate, 0.5% magnesium chloride, 0.1% calcium carbonate media is left to sediment for 2-60 days and the top layer is taken as the production medium) and were grown at 26° C. for 24 hours. 0.1 mL of a 200 mM feed stock solution (5-amino-3-fluorobenzoic acid dissolved in methanol) was added to each falcon tube to give a final feed concentration of 2 mM. Tubes were incubated for a further 6 days at 26° C. The cultures were pooled (approximately 1.4 L) and the falcon tubes were washed (each with 7 mL of water). The washing liquid was pooled (approximately 1.4 L). The pooled cultures and washing liquids were used for isolation of 4,5-dihydro-11-O-desmethyl-15-desmethoxy-18-fluoro-18,21-didesoxymacbecin see below.

7.4 Isolation and characterisation 4,5-dihydro-11-O-desmethyl-15-desmethoxy-18-fluoro-18,21-didesoxymacbecin, 18

The fermentation broth (−3 L) was extracted two times with an equal volume of EtOAc. The organic extracts were combined and the solvent removed in vacuo at 40° C. to yield 3.4 g of an oily residue. This residue was then chromatographed over Silica gel 60 (30×2.5 cm column) with a stepped gradient from 100% CHCl₃ to CHCl₃:MeOH (96:4) and collecting fractions of approx. 250 mL. The fractions were monitored by analytical HPLC. Fractions containing 18 were combined and solvent removed in vacuo at 40° C. to yield 528 mg of semi-pure 18. This semi-pure material was further purified by reversed-phase HPLC over a Phenomenex-Luna C₁₈-BDS column (21.2×250 mm, 5 micron particle size) eluting with a gradient of water:acetonitrile, (77:23) to (20:80), over 25 min at a flow rate of 21 mL/min. 18 eluted at 20 min and the relevant fractions were combined, the solvent removed at reduced pressure to yield 18 as a white powder (224 mg). NMR data acquired in d₆-acetone were entirely consistent with the reported structure.

The purity of 18 was confirmed as described in General Methods using LCMS method 2. Measurements were taken at multiple wavelengths and using MS analysis in both positive and negative modes. LCMS: 18, RT=11.9 min ([M−H]⁻, m/z=503.1; [M+Na]⁺, m/z=527.2; [2M+Na]⁺, 1031.5).

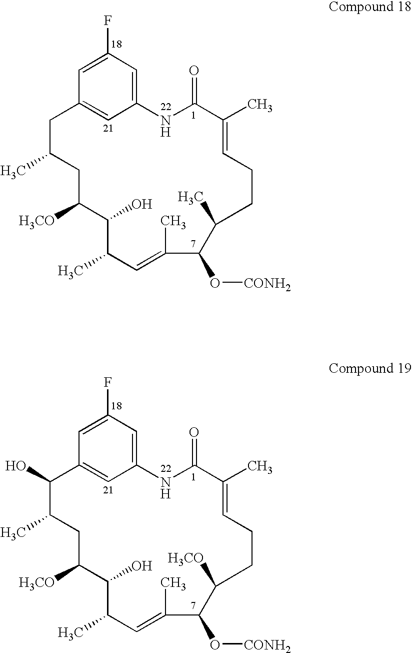

### Example 8

### Generation of 4,5-dihydro-11-O-desmethyl-15-desmethoxy-18,21-didesoxy-17,18,21-trifluoromacbecin by feeding 5-amino-2,3,6-tri-fluorobenzoic acid to BIOT-3870

8.1 Biotransformation of 5-amino-2,3,6-tri-fluorobenzoic acid with BIOT-3870

BIOT-3870 was patched onto MAM plates (medium 4) and grown at 28° C. for three days. A 6 mm circular plug was used to inoculate individual 50 mL falcon tubes containing 10 mL seed medium (adapted from medium 1-2% glucose, 3% soluble starch, 0.5% corn steep solids, 1% soybean flour, 0.5% peptone, 0.3% sodium chloride, 0.5% calcium carbonate). These seed cultures were incubated for 65 hours at 28° C., 200 rpm with a 2 inch throw. These were then used to inoculate (1 mL into 10 mL) modified production medium (adapted from medium 2-5% glycerol, 1% corn steep solids, 2% yeast extract, 2% potassium dihydrogen phosphate, 0.5% magnesium chloride, 0.1% calcium carbonate media is left to sediment for 2-60 days and the top layer is taken as the production medium) and were grown at 26° C. for 24 hours. 0.1 mL of a 200 mM feed stock solution (5-amino-2,3,6-tri-fluorobenzoic acid dissolved in methanol) was added to each falcon tube to give a final feed concentration of 2 mM. Tubes were incubated for a further 6 days at 26° C.

8.2 Identification of 4,5-dihydro-11-O-desmethyl-15-desmethoxy-18,21-didesoxy-17,18,21-trifluoromacbecin, 20

Analysis was performed as described in General Methods using LCMS method 1. In addition to 14 and 15 a new compound was observed with LCMS charateristics described in Table 9. These data were consistent with the title compound.

TABLE 9

| | Retention | | | |
|---|---|---|---|---|
| Compound | time (min) | [M + Na]⁺, m/z | [M − H]⁻, m/z | Mass |
| 14 | 11.5 | 525.2 | 501.2 | 502 |
| 15 | 9.9 | 541.1 | 517.1 | 518 |
| 20 | 13.2 | Not observed | 539.2 | 540 |

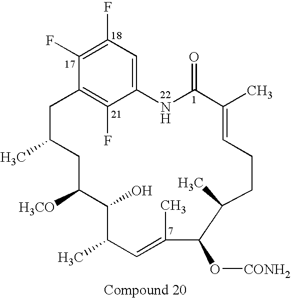

### Example 9

### Generation of an Actinosynnema pretiosum strain in which mbcM has an In-Frame Deletion and mbcMT1, mbcMT2, mbcP and mbcP450 have Additionally been Deleted

9.1 Cloning of DNA Homologous to the Downstream Flanking Region of mbcMT2

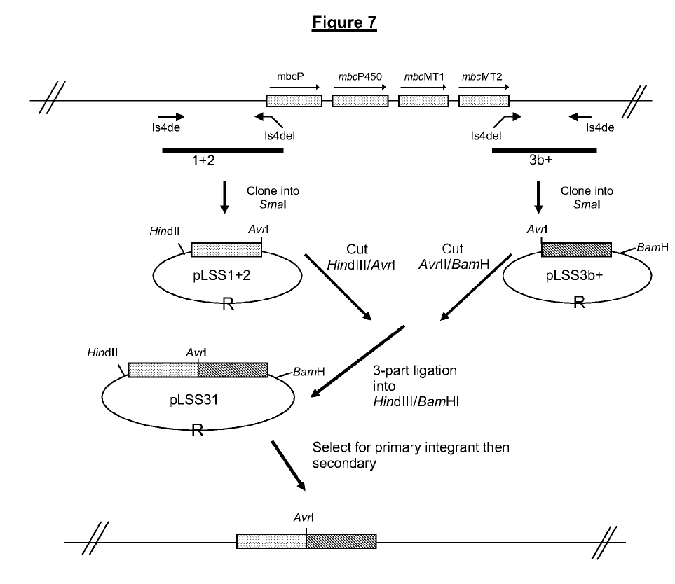

Oligos ls4del1 (SEQ ID NO: 23) and ls4del2a (SEQ ID NO: 24) were used to amplify a 1595 bp region of DNA from Actinosynnema pretiosum (ATCC 31280) in a standard PCR reaction using cosmid 52 (from example 1) as the template and Pfu DNA polymerase. A 5′ extension was designed in oligo ls4del2a to introduce an AvrII site to aid cloning of the amplified fragment (FIG. 7). The amplified PCR product (1+2a, FIG. 8 SEQ ID NO: 25) encoded 196 bp of the 3′ end of mbcMT2 and a further 1393 bp of downstream homology. This 1595 bp fragment was cloned into pUC19 that had been linearised with SmaI, resulting in plasmid pLSS1+2a.

Is4del1

(SEQ ID NO: 23)

5′-GGTCACTGGCCGAAGCGCACGGTGTCATGG-3′
Is4del2a

(SEQ ID NO: 24)

5′-CCTAGGCGACTACCCCGCACTACTACACCGAGCAGG-3′

9.2 Cloning of DNA Homologous to the Upstream Flanking Region of mbcM.

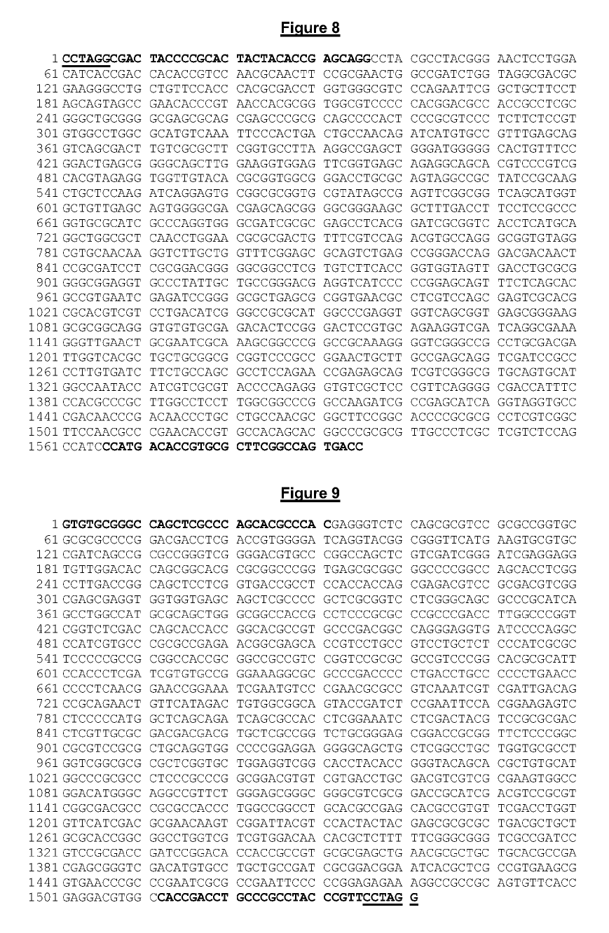

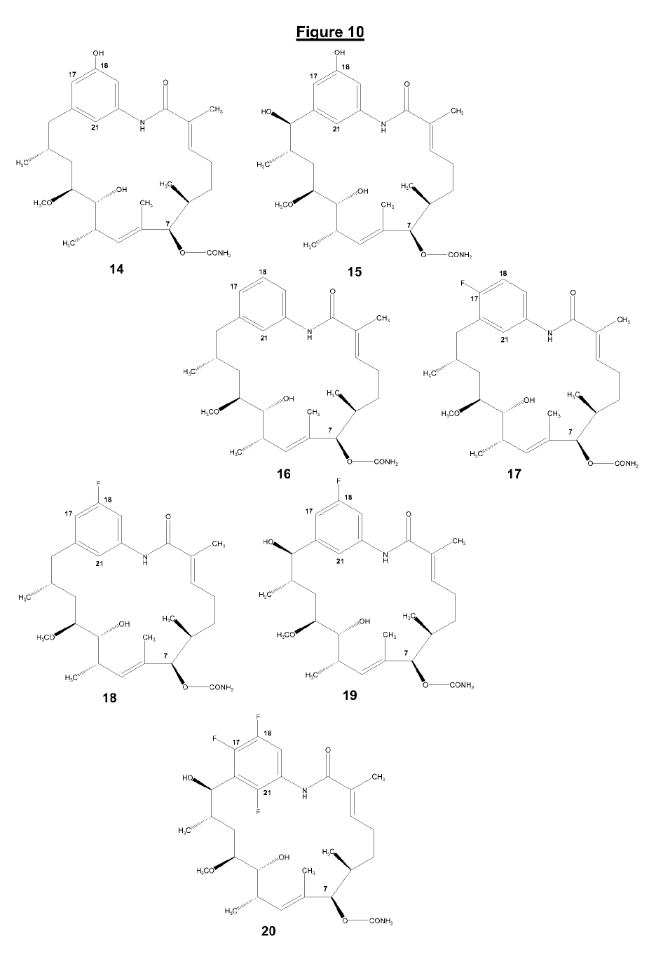

Oligos ls4del3b (SEQ ID NO: 26) and ls4del4 (SEQ ID NO: 27) were used to amplify a 1541 bp region of DNA from Actinosynnema pretiosum (ATCC 31280) in a standard PCR reaction using cosmid 52 (from example 1) as the template and Pfu DNA polymerase. A 5′ extension was designed in oligo ls4del3b to introduce an AvrII site to aid cloning of the amplified fragment (FIG. 7). The amplified PCR product (3b+4, FIG. 9, SEQ ID NO: 28) encoded 100 bp of the 5′ end of mbcP and a further ˜1450 bp of upstream homology. This ˜1550 bp fragment was cloned into pUC19 that had been linearised with SmaI, resulting in plasmid pLSS3b+4.

Is4del3b

(SEQ ID NO: 26)

5′-CCTAGGAACGGGTAGGCGGGCAGGTCGGTG-3′
Is4del4

(SEQ ID NO: 27)

5′-GTGTGCGGGCCAGCTCGCCCAGCACGCCCAC-3′

The products 1+2a and 3b+4 were cloned into pUC19 to utilise the HindIII and BamHI sites in the pUC19 polylinker for the next cloning step.

The 1621 bp AvrII/HindIII fragment from pLSS1+2a and the 1543 bp AvrII/BamHI fragment from pLSS3b+4 were cloned into the 3556 bp HindIII/BamHI fragment of pKC1132 to make pLSS315. pLSS315 therefore contained a HindIII/BamHI fragment encoding DNA homologous to the flanking regions of the desired four ORF deletion region fused at an AvrII site (FIG. 7).

9.3 Transformation of BIOT-3870 with pLSS315

Escherichia coli ET12567, harbouring the plasmid pUZ8002 was transformed with pLSS315 by electroporation to generate the E. coli donor strain for conjugation. This strain was used to transform BIOT-3870 by vegetative conjugation (Matsushima et al, 1994). Exconjugants were plated on MAM medium (1% wheat starch, 0.25% corn steep solids, 0.3% yeast extract, 0.3% calcium carbonate, 0.03% iron sulphate, 2% agar) and incubated at 28° C. Plates were overlayed after 24 h with 50 mg/L apramycin and 25 mg/L nalidixic acid. As pLSS315 is unable to replicate in BIOT-3870, apramycin resistant colonies were anticipated to be transformants that contained plasmid integrated into the chromosome by homologous recombination via the plasmid borne regions of homology.

### 9.4 Screening for Secondary Crosses

Three primary transformants of BIOT-3870:pLSS315 were selected for subculturing to screen for secondary crosses.

Strains were patched onto MAM media (supplemented with 50 mg/L apramycin) and grown at 28° C. for four days. Two 6 mm circular plugs were used to inoculate 30 mL of ISP2 (0.4% yeast extract, 1% malt extract, 0.4% dextrose, not supplemented with antibiotic) in a 250 ml conical flask. Cultures were grown for 2-3 days then subcultured (5% inoculum) into 30 mL of ISP2 in a 250 ml conical flask. After 4-5 rounds of subculturing the cultures were protoplasted as described in Example 3.6, the protoplasts were serially diluted, plated on regeneration media (see Example 3.6) and incubated at 28° C. for four days. Single colonies were then patched in duplicate onto MAM media containing apramycin and onto MAM media containing no antibiotic and the plates were incubated at 28° C. for four days. Seven patches derived from clone no 1 (no 32-37) and four patches derived from clone no 3 (no 38-41) that grew on the no antibiotic plate but did not grow on the apramycin plate were re-patched onto +/−apramycin plates to confirm that they had lost the antibiotic marker.

Production of macbecin analogues was carried out as described in the General Methods. Analysis was performed as described in General Methods using LCMS method 1. Compound 14 was produced in yields comparable to the parent strain BIOT-3870 and no production of compound 15 was observed for patches 33, 34, 35, 37, 39 and 41. This result shows that the desired mutant strains have a deletion of 3892 bp of the macbecin cluster containing the genes mbcP, mbcP450, mbcMT1 and mbcMT2 in addition to the original deletion of mbcM.

### Example 10

### Binding to Hsp90

### Isothermal Titration Carorimetry and K_{d} Determinations.

Yeast Hsp90 was dialysed against 20 mM Tris pH 7.5 containing 1 mM EDTA and 5 mM NaCl and then diluted to 0.008 mM in the same buffer, but containing 2% DMSO. The test compounds were dissolved in 100% DMSO at a concentration of 50 mM and subsequently diluted to 0.1 mM in the same buffer as for Hsp90 with 2% DMSO. Heats of interaction were measured at 30° C. on a MSC system (Microcal), with a cell volume of 1.458 mL. 10 aliquots of 0.027 mL of 0.100 mM of each test compound were injected into 0.008 mM yeast Hsp90. Heats of dilution were determined in a separate experiment by injecting the test compound into buffer containing 2% DMSO, and the corrected data fitted using a nonlinear least square curve-fitting algorithm (Microcal Origin) with three floating variables: stoichiometry, binding constant and change in enthalpy of interaction. The results are shown below in Table 10.

TABLE 10
Kd values for Hsp90 binding

| | Kd (nM) |
|---|---|
| macbecin | 240 |
| 16 | 20 |
| 17 | 19 |
| 18 | 23.5 |
| Geldanamycin | 1200 |

### Example 11

### Biological Data—In Vitro Evaluation of Anticancer Activity of 18,21-didesoxymacbecin Analogues

In vitro evaluation of the test compounds for anticancer activity in a panel of human tumour cell lines in a monolayer proliferation assay was carried out as described in the general methods using a modified propidium iodide assay.

The results are displayed in Table 11 below, all treated/control (% T/C) values shown are the average of at least 3 separate experiments. Table 12 shows the mean IC₇₀ for the compounds across the cell line panel tested, with macbecin shown as a reference (where the mean is calculated as the geometric mean of all replicates).

TABLE 11
in vitro cell line data

| | Test/Control (%) at drug concentration (μg/mL) | | | | | | | | | | | | | | |
|---|---|---|---|---|---|---|---|---|---|---|---|---|---|---|---|
| | Compound 16 | | | | | Compound 17 | | | | | Compound 18 | | | | |
| Cell line | 0.01 | 0.1 | 1 | 10 | 100 | 0.01 | 0.1 | 1 | 10 | 100 | 0.01 | 0.1 | 1 | 10 | 100 |
| CNXF | 100 | 19 | 8 | 8 | 6 | 97 | 22 | 7 | 8 | 7 | 64 | 9 | 9 | 11 | 9 |
| 498NL | | | | | | | | | | | | | | | |
| CXF | 106 | 52 | 8 | 7 | 6 | 102 | 49 | 7 | 7 | 8 | 98 | 14 | 11 | 13 | 11 |
| HT29 | | | | | | | | | | | | | | | |
| LXF | 106 | 48 | 10 | 7 | 5 | 100 | 41 | 10 | 11 | 11 | 94 | 43 | 13 | 13 | 12 |
| 1121L | | | | | | | | | | | | | | | |
| MCF-7 | 83 | 21 | 10 | 11 | 10 | 86 | 20 | 10 | 10 | 8 | 77 | 27 | 12 | 12 | 8 |
| MEXF | 100 | 64 | 7 | 5 | 3 | 101 | 21 | 4 | 3 | 3 | 91 | 24 | 7 | 5 | 4 |
| 394NL | | | | | | | | | | | | | | | |
| DU145 | 96 | 47 | 8 | 8 | 8 | 93 | 7 | 8 | 10 | 9 | 66 | 9 | 8 | 9 | 6 |

TABLE 12
average IC₇₀ value across the cell-line panel

| | IC₇₀ (μg/mL) |
|---|---|
| macbecin | 0.21 |
| 16 | 0.193 |
| 17 | 0.106 |
| 18 | 0.077 |

All references including patent and patent applications referred to in this application are incorporated herein by reference to the fullest extent possible.

Throughout the specification and the claims which follow, unless the context requires otherwise, the word ‘comprise’, and variations such as ‘comprises’ and ‘comprising’, will be understood to imply the inclusion of a stated integer or step or group of integers but not to the exclusion of any other integer or step or group of integers or steps.

### REFERENCES

Allen, I. W. and Ritchie, D. A. (1994) Cloning and analysis of DNA sequences from Streptomyces hygroscopicus encoding geldanamycin biosynthesis. Mol. Gen. Genet. 243: 593-599.
Bagatell, R. and Whitesell, L. (2004) Altered Hsp90 function in cancer: A unique therapeutic opportunity. Molecular Cancer Therapeutics 3: 1021-1030.
Beliakoff, J. and Whitesell, L. (2004) Hsp90: an emerging target for breast cancer therapy. Anti-Cancer Drugs 15:651-662.
Bierman, M., Logan, R., O'Brien, K., Seno, E T., Nagaraja Rao, R. and Schoner, B E. (1992) “Plasmid cloning vectors for the conjugal transfer of DNA from Escherichia coli to Streptomyces spp.” Gene 116: 43-49.
Blagosklonny, M. V. (2002) Hsp-90-associated oncoproteins: multiple targets of geldanamycin and its analogues. Leukemia 16:455-462.
Blagosklonny, M. V., Toretsky, J., Bohen, S, and Neckers, L. (1996) Mutant conformation of p53 translated in vitro or in vivo requires functional HSP90. Proc. Natl. Acad. Sci. USA 93:8379-8383.
Bohen, S. P. (1998) Genetic and biochemical analysis of p23 and ansamycin antibiotics in the function of Hsp90-dependent signaling proteins. Mol Cell Biol 18:3330-3339.
Carreras, C. W., Schirmer, A., Zhong, Z. and Santi D. V. (2003) Filter binding assay for the geldanamycin-heat shock protein 90 interaction. Analytical Biochemistry 317:40-46.
Cassady, J. M., Chan, K. K., Floss, H. G. and Leistner E. (2004) Recent developments in the maytansinoid antitumour agents. Chem. Pharm. Bull. 52(1) 1-26.
Chiosis, G., Huezo, H., Rosen, N., Mimnaugh, E., Whitesell, J. and Neckers, L. (2003) 17AAG: Low target binding affinity and potent cell activity—finding an explanation. Molecular Cancer Therapeutics 2:123-129.
Chiosis, G., Vilenchik, M., Kim, J. and Solit, D. (2004) Hsp90: the vulnerable chaperone. Drug Discovery Today 9:881-888.
Csermely, P. and Soti, C. (2003) Inhibition of Hsp90 as a special way to inhibit protein kinases. Cell. Mol. Biol. Lett. 8:514-515.
DeBoer, C and Dietz, A. (1976) The description and antibiotic production of Streptomyces hygroscopicus var. geldanus. J. Antibiot. 29:1182-1188.
DeBoer, C., Meulman, P. A., Wnuk, R. J., and Peterson, D. H. (1970) Geldanamycin, a new antibiotic. J. Antibiot. 23:442-447.
Dengler W. A., Schulte J., Berger D. P., Mertelsmann R. and Fiebig H H. (1995) Development of a propidium iodide fluorescence assay for proliferation and cytotoxicity assay. Anti-Cancer Drugs, 6:522-532.
Dikalov, s., Landmesser, U., Harrison, D G., 2002, Geldanamycin Leads to Superoxide Formation by Enzymatic and Non-enzymatic Redox Cycling, The Journal of Biological Chemistry, 277(28), pp 25480-25485
Donzê O. and Picard, D. (1999) Hsp90 binds and regulates the ligand-inducible a subunit of eukaryotic translation initiation factor kinase Gcn2. Mol Cell Biol 19:8422-8432.
Egorin M J, Lagattuta T F, Hamburger D R, Covey J M, White K D, Musser S M, Eiseman J L. (2002) “Pharmacokinetics, tissue distribution, and metabolism of 17-(dimethylaminoethylamino)-17-demethoxygeldanamycin (NSC 707545) in CD2F1 mice and Fischer 344 rats.” Cancer Chemother Pharmacol, 49(1), pp 7-19.
Eustace, B. K., Sakurai, T., Stewart, J. K., et al. (2004) Functional proteomic screens reveal an essential extracellular role for hsp90α in cancer cell invasiveness. Nature Cell Biology 6:507-514.
Fang, Y., Fliss, A. E., Rao, J. and Caplan A. J. (1998) SBAI encodes a yeast Hsp90 cochaperone that is homologous to vertebrate p23 proteins. Mol Cell Biol 18:3727-3734.
Fiebig H. H., Dengler W. A. and Roth T. Human tumor xenografts: Predictivity, characterization, and discovery of new anticancer agents. In: Fiebig H H, Burger A M (eds). Relevance of Tumor Models for Anticancer Drug Development. Contrib. Oncol. 1999, 54: 29-50.
Goetz, M. P., Toft, D. O., Ames, M. M. and Ehrlich, C. (2003) The Hsp90 chaperone complex as a novel target for cancer therapy. Annals of Oncology 14:1169-1176.
Harris, S. F., Shiau A. K. and Agard D. A. (2004) The crystal structure of the carboxy-terminal dimerization domain of htpG, the Escherichia coli Hsp90, reveals a potential substrate binding site. Structure 12: 1087-1097.
Hong, Y.-S., Lee, D., Kim, W., Jeong, J.-K. et al. (2004) Inactivation of the carbamoyltransferase gene refines post-polyketide synthase modification steps in the biosynthesis of the antitumor agent geldanamycin. J. Am. Chem. Soc. 126:11142-11143.
Hostein, I., Robertson, D., DiStefano, F., Workman, P. and Clarke, P.A. (2001) Inhibition of signal transduction by the Hsp90 inhibitor 17-allylamino-17-demethoxygeldanamycin results in cytostasis and apoptosis. Cancer Research 61:4003-4009.
Hu, Z., Liu, Y., Tian, Z.-Q., Ma, W., Starks, C. M. et al. (2004) Isolation and characterization of novel geldanamycin analogues. J. Antibiot. 57:421-428.
Hur, E., Kim, H.-H., Choi, S. M., et al. (2002) Reduction of hypoxia-induced transcription through the repression of hypoxia-inducible factor-1α/aryl hydrocarbon receptor nuclear translocator DNA binding by the 90-kDa heat-shock protein inhibitor radicicol. Molecular Pharmacology 62:975-982.
Iwai Y, Nakagawa, A., Sadakane, N., Omura, S., Oiwa, H., Matsumoto, S., Takahashi, M., Ikai, T., Ochiai, Y. (1980) Herbimycin B, a new benzoquinoid ansamycin with anti-TMV and herbicidal activities. The Journal of Antibiotics, 33(10), pp 1114-1119.
Jez, J. M., Chen, J. C.-H., Rastelli, G., Stroud, R. M. and Santi, D. V. (2003) Crystal structure and molecular modeling of 17-DMAG in complex with human Hsp90. Chemistry and Biology 10:361-368.
Kaur, G., Belotti, D, Burger, A. M., Fisher-Nielson, K., Borsotti, P. et al. (2004) Antiangiogenic properties of 17-(Dimethylaminoethylamino)-17-Demethoxygeldanamycin: an orally bioavailable heat shock protein 90 modulator. Clinical Cancer Research 10:4813-4821.
Kieser, T., Bibb, M. J., Buttner, M. J., Chater, K. F., and Hopwood, D. A. (2000) Practical Streptomyces Genetics, John Innes Foundation, Norwich Kumar, R., Musiyenko, A. and Barik S. (2003) The heat shock protein 90 of Plasmodium falciparum and antimalarial activity of its inhibitor, geldanamycin. J Malar 2:30.
Kurebayashi, J., Otsuke, T., Kurosumi, M., Soga, S., Akinaga, S, and Sonoo, H. (2001) A radicicol derivative, KF58333, inhibits expression of hypoxia-inducible factor-1α and vascular endothelial growth factor, angiogenesis and growth of human breast cancer xenografts. Jpn. J. Cancer Res. 92:1342-1351.
Le Brazidec, J.-Y., Kamal, A., Busch, D., Thao, L., Zhang, L. et al. (2003) Synthesis and biological evaluation of a new class of geldanamycin derivatives as potent inhibitors of Hsp90. J. Med. Chem. 47: 3865-3873.
Lee, Y.-S., Marcu, M. G. and Neckers, L. (2004) Quantum chemical calculations and mutational analysis suggest heat shock protein 90 catalyzes trans-cis isomeration of geldanamycin. Chem. Biol. 11:991-998.
Liu, X.-D., Morano, K. A. and Thiele D. J. (1999); The yeast Hsp 110 family member, Sse1, is an Hsp90 cochaperone. J Biol Chem 274:26654-26660.
Mandler, R., Wu, C., Sausville, E. A., Roettinger, A. J., Newman, D. J., Ho, D. K., King, R., Yang, D., Lippman, M. E., Landolfi, N. F., Dadachova, E., Brechbiel, M. W. and Waldman, T. A. (2000) Immunoconjugates of geldanamycin and anti-HER2 monoclonal antibodies: antiproliferative activity on human breast carcinoma cell lines. Journal of the National Cancer Institute 92:1573-1581.
Matsushima, P., M. C. Broughton, et al. (1994). Conjugal transfer of cosmid DNA from Escherichia coli to Saccharopolyspora spinosa: effects of chromosomal insertions on macrolide A83543 production. Gene 146(1): 39-45.
McLaughlin S. H., Smith, H. W. and Jackson S. E. (2002) Stimulation of the weak ATPase activity of human Hsp90 by a client protein. J. Mol. Biol. 315: 787-798.
McCammon, M. T. and L. W. Parks (1981). Inhibition of sterol transmethylation by S-adenosylhomocysteine analogs. J Bacteriol 145(1): 106-12.
Muroi M, Izawa M, Kosai Y, Asai M. (1981) “The structures of macbecin 1 and 11” Tetrahedron, 37, pp 1123-1130.

Muroi, M., Izawa M., Kosai, Y., and Asai, M. (1980) Macbecins 1 and 11, New Antitumor antibiotics. II. Isolation and characterization. J Antibiotics 33:205-212.
Neckers, L (2003) Development of small molecule Hsp90 inhibitors: utilizing both forward and reverse chemical genomics for drug identification. Current Medicinal Chemistry 9:733-739.
Neckers, L. (2002) Hsp90 inhibitors as novel cancer chemotherapeutic agents. Trends in Molecular Medicine 8:S55-S61.
Nimmanapalli, R., O'Bryan, E., Kuhn, D., Yamaguchi, H., Wang, H.-G. and Bhalla, K. N. (2003) Regulation of 17-AAG-induced apoptosis: role of Bcl-2, Bcl-X_{L}, and Bax downstream of 17-AAG-mediated down-regulation of Akt, Raf-1, and Src kinases. Neoplasia 102:269-275.
Omura, S., Iwai, Y., Takahashi, Y., Sadakane, N., Nakagawa, A., Oiwa, H., Hasegawa, Y., Ikai, T., (1979), Herbimycin, a new antibiotic produced by a strain of Streptomyces. The Journal of Antibiotics, 32(4), pp 255-261.
Omura, S., Miyano, K., Nakagawa, A., Sano, H., Komiyama, K., Umezawa, I., Shibata, K, Satsumabayashi, S., (1984), “Chemical modification and antitumor activity of Herbimycin A. 8,9-epoxide, 7,9-carbamate, and 17 or 19-amino derivatives”. The Journal of Antibiotics, 37(10), pp 1264-1267.
Ono, Y., Kozai, Y. and Ootsu, K. (1982) Antitumor and cytocidal activities of a newly isolated benzenoid ansamycin, Macbecin I. Gann. 73:938-44.
Patel, K., M. Piagentini, Rascher, A., Tian, Z. Q., Buchanan, G. O., Regentin, R., Hu, Z., Hutchinson, C. R. And McDaniel, R. (2004). “Engineered biosynthesis of geldanamycin analogs for hsp90 inhibition.” Chem Biol 11(12): 1625-33.
Pfeifer, B. A. and C. Khosla (2001). “Biosynthesis of polyketides in heterologous hosts.” Microbiology and Molecular Biology Reviews 65(1): 106-118.
Rascher, A., Hu, Z., Viswanathan, N., Schirmer, A. et al. (2003) Cloning and characterization of a gene cluster for geldanamycin production in Streptomyces hygroscopicus NRRL 3602. FEMS Microbiology Letters 218:223-230.
Rascher, A., Z. Hu, Buchanan, G. O., Reid, R. and Hutchinson, C. R. (2005). Insights into the biosynthesis of the benzoquinone ansamycins geldanamycin and herbimycin, obtained by gene sequencing and disruption. Appl Environ Microbiol 71(8): 4862-71.
Rawlings, B. J. (2001). “Type I polyketide biosynthesis in bacteria (Part B).” Natural Product Reports 18(3): 231-281.
Roth T., Burger A. M., Dengler W., Willmann H. and Fiebig H. H. Human tumor cell lines demonstrating the characteristics of patient tumors as useful models for anticancer drug screening. In: Fiebig H H, Burger A M (eds). Relevance of Tumor Models for Anticancer Drug Development. Contrib. Oncol. 1999, 54: 145-156.
Rowlands, M. G., Newbatt, Y. M., Prodromou, C., Pearl, L. H., Workman, P. and Aherne, W. (2004) High-throughput screening assay for inhibitors of heat-shock protein 90 ATPase activity. Analytical Biochemistry 327:176-183
Schulte, T. W., Akinaga, S., Murakata, T., Agatsuma, T. et al., (1999) Interaction of radicicol with members of the heat shock protein 90 family of molecular chaperones. Molecular Endocrinology 13:1435-1488.
Shibata, K., Satsumabayashi, S., Nakagawa, A., Omura, S. (1986a) The structure and cytocidal activity of herbimycin C. The Journal of Antibiotics, 39(11), pp 1630-1633.
Shibata, K., Satsumabayashi, S., Sano, H., Komiyama, K., Nakagawa, A., Omura, S. (1986b)
Chemical modification of Herbimycin A: synthesis and in vivo antitumor activities of halogenated and other related derivatives of herbimycin A. The Journal of Antibiotics, 39(3), pp 415-423.
Shirling, E. B. and Gottlieb, D. (1966) International Journal of Systematic Bacteriology 16:313-340
Smith-Jones, P. M., Solit, D. B., Akhurst, T., Afroze, F., Rosen, N. and Larson, S. M. (2004) Imaging the pharmacodynamics of HER2 degradation in response to Hsp90 inhibitors. Nature Biotechnology 22:701-706.
Spiteller, P., Bai, L., Shang, G., Carroll, B. J., Yu, T.-W. and Floss, H. G. (2003). The post-polyketide synthase modification steps in the biosynthesis of the antitumor agent ansamitocin by Actinosynnema pretiosum. J Am Chem Soc 125(47): 14236-7
Sreedhar A. S., Nardai, G. and Csermely, P. (2004) Enhancement of complement-induced cell lysis: a novel mechanism for the anticancer effects of Hsp90 inhibitors. Immunology letters 92:157-161.
Sreedhar, A. S., Soti, C. and Csermely, P. (2004a) Inhibition of Hsp90: a new strategy for inhibiting protein kinases. Biochimica Biophysica Acta 1697:233-242.
Staunton, J. and K. J. Weissman (2001). “Polyketide biosynthesis: a millennium review.” Natural Product Reports 18(4): 380-416.
Stead, P., Latif, S., Blackaby, A. P. et al. (2000) Discovery of novel ansamycins possessing potent inhibitory activity in a cell-based oncostatin M signalling assay. J Antibiotics 53:657-663.
Supko, J. G., Hickman, R. L., Grever, M. R. and Malspeis, L (1995) Preclinical pharmacologic evaluation of geldanamycin as an antitumor agent. Cancer Chemother. Pharmacol. 36:305-315.
Takahashi, A., Casais, C., Ichimura K. and Shirasu, K. (2003) HSP90 interacts with RAR1 and SGT1 and is essential for RPS2-mediated disease resistance in Arabidopsis. Proc. Natl. Acad. Sci. USA 20:11777-11782.
Tanida, S., Hasegawa, T. and Higashide E. (1980) Macbecins I and II, New Antitumor antibiotics. I. Producing organism, fermentation and antimicrobial activities. J Antibiotics 33:199-204.
Tian, Z.-Q., Liu, Y., Zhang, D., Wang, Z. et al. (2004) Synthesis and biological activities of novel 17-aminogeldanamycin derivatives. Bioorganic and Medicinal Chemistry 12:5317-5329. Uehara, Y. (2003) Natural product origins of Hsp90 inhibitors. Current Cancer Drug Targets 3:325-330.
Vasilevskaya, I. A., Rakitina, T. V. and O'Dwyer, P. J. (2003) Geldanamycin and its 17-Allylamino-17-Demethoxy analogue antagonize the action of cisplatin in human colon adenocarcinoma cells: differential caspase activation as a basis of interaction. Cancer Research 63: 3241-3246.
Watanabe, K., Okuda, T., Yokose, K., Furumai, T. and Maruyama, H. H. (1982) Actinosynnema mirum, a new producer of nocardicin antibiotics. J. Antibiot. 3:321-324.
Weber, J. M., Losick, R. (1988) The use of a chromosome integration vector to a map erythromycin resistance and production genes in Sacharopolyspora erythraea (Streptomyces erythraeus) Gene 68(2), 173-180
Wegele, H., Müller, L. and Buchner, J. (2004) Hsp70 and Hsp90-a relay team for protein folding. Rev Physiol Biochem Pharmacol 151:1-44.
Wenzel, S. C., Gross, F, Zhang, Y., Fu, J., Stewart, A. F. and Müller, R (2005) Heterologous expression of a myxobacterial natural products assembly line in Pseudomonads via Red/ET recombineering. Chemistry & Biology 12: 249-356.
Whitesell, L., Mimnaugh, E. G., De Costa, B., Myers, C. E. and Neckers, L. M. (1994) Inhibition of heat shock protein HSP90-pp60^{v-src} heteroprotein complex formation by benzoquinone ansamycins: Essential role for stress proteins in oncogenic transformation. Proc. Natl. Acad. Sci. USA 91: 8324-8328.
Winklhofer, K. F., Heller, U., Reintjes, A. and Tatzelt J. (2003) Inhibition of complex glycosylation increases the formation of PrPsc. Traffic 4:313-322.
Workman P. (2003) Auditing the pharmacological accounts for Hsp90 molecular chaperone inhibitors: unfolding the relationship between pharmacokinetics and pharmacodynamics. Molecular Cancer Therapeutics 2:131-138.
Workman, P. and Kaye, S. B. (2002) Translating basic cancer research into new cancer therapeutics. Trends in Molecular Medicine 8:S1-S9.
Young, J. C.; Moarefi, I. and Hartl, U. (2001) Hsp90: a specialized but essential protein folding tool. J. Cell. Biol. 154:267-273.

The application, of which this description and claims form part, may be used as a basis for priority in respect of any subsequent application. The claims of such subsequent application may be directed to any feature or combination of features described herein. They may take the form of product, composition, process, or use claims and may include, by way of example and without limitation, the following claims:

1: An 18,21-didesoxymacbecin analogue according to the formula (I) below, or a pharmaceutically acceptable salt thereof: 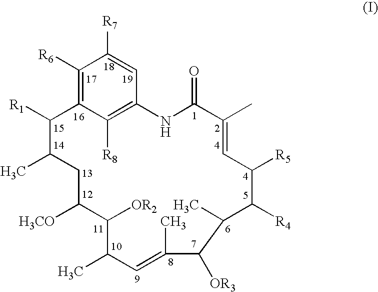
wherein:
R₁ represents H, OH, OMe;
R₂ represents H or Me;
R₃ represents H or CONH₂;
R₄ and R₅ either both represent H or together they represent a bond (i.e. C4 to C5 is a double bond);
R₆ represents H or F;
R₇ represents H or F; and
R₈represents H or F.

2: The compound according to claim 1, wherein R₁ represents H.

3: The compound according to claim 1, wherein R₁ represents OH.

4: The compound according to claim 1, wherein R₂ represents H.

5: The compound according to claim 1, wherein R₃ represents CONH₂.

6: The compound according to claim 1, wherein R₄ and R₅ together represent a bond.

7: The compound according to claim 1, wherein R₄ and R₅ each represent H.

8: The compound according to claim 1 wherein R₆, R₇ and R₈ all represent H.

9: The compound according to claim 1 wherein R₆, R₇ and R₈ do not all represent H.

10: The compound according to claim 1, wherein R₁ represents H, R₂ represents H and R₃ represents CONH₂ and R₄ and R₅ each represent H.

11: The compound according to claim 1, wherein R₁ represents OH, R₂ represents H and R₃ represents CONH₂ and R₄ and R₅ each represent H.

12: The compound according to claim 1, wherein R₁ represents H, R₂ represents H, R₃ represents CONH₂, R₄ and R₅ each represent H, R₆, R₇ and R₈ each represent H.

13: The compound according to claim 1, wherein R₁ represents OH, R₂ represents H, R₃ represents CONH₂, R₄ and R₅ each represent H, R₆, R₇ and R₈ each represent H.

14: The compound according to claim 1, wherein R₁ represents H, R₂ represents H, R₃ represents CONH₂, R₄ and R₅ each represent H, R₆ represents F and R₇ and R₈ each represent H.

15: The compound according to claim 1, wherein R₁ represents OH, R₂ represents H, R₃ represents CONH₂, R₄ and R₅ each represent H, R₆ represents F and R₇ and R₈ each represent H.

16: The compound according to claim 1, wherein R₁ represents H, R₂ represents H, R₃ represents CONH₂, R₄ and R₅ each represent H, R₆ represents H, R₇ represents F and R₈ represents H.

17: The compound according to claim 1, wherein R₁ represents OH, R₂ represents H, R₃ represents CONH₂, R₄ and R₅ each represent H, R₆ represents H, R₇ represents F and R₈ represents H.

18: The compound according to claim 1, wherein R₁ represents H, R₂ represents H, R₃ represents CONH₂, R₄ and R₅ each represent H, R₆ and R₇ each represent F and R₈ represents H.

19: The compound according to claim 1, wherein R₁ represents OH, R₂ represents H, R₃ represents CONH₂, R₄ and R₅ each represent H, R₆ and R₇ each represent F and R₈ represents H.

20: The compound according to claim 1, wherein R₁ represents H, R₂ represents H, R₃ represents CONH₂, R₄ and R₅ each represent H, R₆, R₇ and R₈ each represent F.

21: The compound according to claim 1 which is 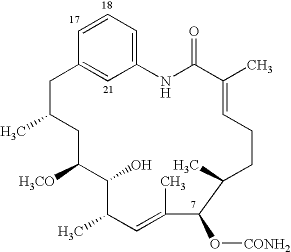
or a pharmaceutically acceptable salt thereof.

22: The compound according to claim 1 which is 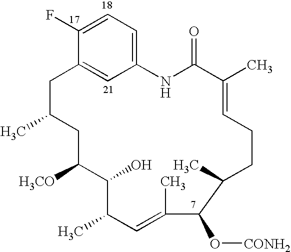
or a pharmaceutically acceptable salt thereof.

23: The compound according to claim 1 which is 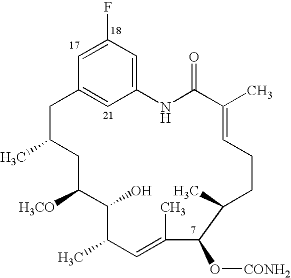
or a pharmaceutically acceptable salt thereof.

24: The compound according to claim 1 which is 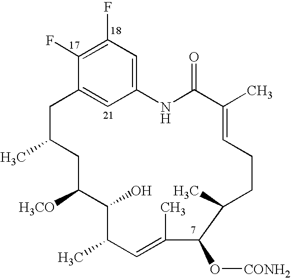
or a pharmaceutically acceptable salt thereof.

25: The compound according to claim 1 which is 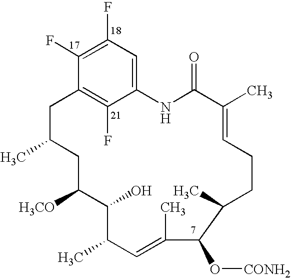
or a pharmaceutically acceptable salt thereof.

26: The compound according to claim 1 which is 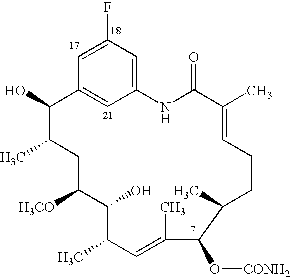
or a pharmaceutically acceptable salt thereof.

27: A pharmaceutical composition comprising an 18,21-didesoxymacbecin analogue or a pharmaceutically acceptable salt thereof according to claim 1, together with one or more pharmaceutically acceptable diluents or carriers.

28-30. (canceled)

31: A method of treatment of cancer, B-cell malignancies, malaria, fungal infection, diseases of the central nervous system and neurodegenerative diseases, diseases dependent on angiogenesis, autoimmune diseases and/or as a prophylactic pretreatment for cancer which comprises administering to a patient in need thereof an effective amount of an 18,21-didesoxymacbecin analogue or a pharmaceutically acceptable salt thereof according to claim 1.

32: The method according to claim 31, wherein the 18,21-didesoxymacbecin analogue or a pharmaceutically acceptable salt thereof is administered in combination with another treatment.

33: The method according to claim 32 where the other treatment is selected from the group consisting of: methotrexate, leukovorin, prenisone, bleomycin, cyclophosphamide, 5-fluorouracil, paclitaxel, docetaxel, vincristine, vinblastine, vinorelbine, doxorubicin, tamoxifen, toremifene, megestrol acetate, anastrozole, goserelin, anti-HER2 monoclonal antibody (e.g. Herceptin™), capecitabine, raloxifene hydrochloride, EGFR inhibitors, VEGF inhibitors, proteasome inhibitors (e.g. Velcade™), radiotherapy and surgery.

34: The method according to claim 32 where the other treatment is selected from the group consisting of: bleomycin, capecitabine, cisplatin, cytarabine, cyclophosphamide, doxorubicin, 5-fluorouracil, gemcitabine, leucovorin, methotrexate, mitoxantone, the taxanes including paclitaxel and docetaxel, vincristine, vinblastine and vinorelbine; the hormonal therapies, anastrozole, goserelin, megestrol acetate, prenisone, tamoxifen and toremifene; the monoclonal antibody therapies such as trastuzumab (ani-Her2), cetuximab (ant-EGFR) and bevacizumab (anti-VEGF); and protein kinase inhibitors such as imatinib, dasatinib, gefitinib, erlotinib, lapatinib, temsirolimus; the proteasome inhibitors such as bortezomib; histone deacetylase (HDAC) inhibitors such as vorinostat; angiogenesis inhibitors such as sunitinib, sorafenib, lenalidomide, radiotherapy and surgery.

35: The method according to claim 32 where the other treatment is selected from the group consisting of conventional chemotherapeutics such as cisplatin, cytarabine, cyclohexylchloroethylnitrosurea, cyclophosphamide, gemcitabine, Ifosfamid, leucovorin, mitomycin, mitoxantone, oxaliplatin and taxanes including taxol and vindesine; hormonal therapies such as anastrozole, goserelin, megestrol acetate and prenisone; monoclonal antibody therapies such as cetuximab (anti-EGFR); protein kinase inhibitors such as dasatinib, lapatinib; histone deacetylase (HDAC) inhibitors such as vorinostat; angiogenesis inhibitors such as sunitinib, sorafenib, lenalidomide; mTOR inhibitors such as temsirolimus; and imatinib.

36: A method for the production of an 18,21-didesoxymacbecin analogue or a pharmaceutically acceptable salt thereof according to claim 1, said method comprising:
a) providing a first host strain that produces macbecin or an analogue thereof when cultured under appropriate conditions
b) feeding a non-natural starter unit to said strain
c) culturing said host strain under suitable conditions for the production of 18,21-didesoxymacbecin analogues; and
d) optionally isolating the compounds produced.

37: The method according to claim 36 wherein the method additional comprises the step of:
e) deleting or inactivating one or more of the starter unit biosynthesis genes, or a homologue thereof, said step usually occurring prior to step c).

38: The method according to claim 36 wherein the method additional comprises the step of:
f) deleting or inactivating one or more post-PKS genes, said step usually occurring prior to step c).

39: The method of claim 36 wherein the non-natural starter unit of step b) is 3-amino-benzoic acid.

40: The method of claim 36 wherein the non-natural starter unit of step b) is 5-amino-2-fluorobenzoic acid.

41: The method of claim 36 wherein the non-natural starter unit of step b) is 5-amino-3-fluorobenzoic acid.

42: The method of claim 36 wherein the non-natural starter unit of step b) is 5-amino-2,3-di-fluorobenzoic acid.

43: The method of claim 36, wherein the non-natural starter unit of step b) is 5-amino-2,3,6-tri-fluorobenzoic acid.

44: The method according to claim 36 wherein in step (a) the strain is a macbecin producing strain.

45: The method according to claim 36 wherein in step (a) the strain is an engineered strain based on a macbecin producing strain in which one or more of the starter unit biosynthesis genes have been deleted or inactivated.

46: The method according to claim 36 wherein in step (a) the strain is an engineered strain based on a macbecin producing strain in which one or more of the post-PKS genes have been deleted or inactivated.

47: The method according to claim 46 wherein in step (a) the strain is an engineered strain based on a macbecin producing strain in which mbcM and optionally further post-PKS genes have been deleted or inactivated.

48: The method according to claim 47 wherein in step (a) the strain is an engineered strain based on a macbecin producing strain in which mbcM has been deleted or inactivated.

49: The method according to claim 47 wherein in step (a) the strain is an engineered strain based on a macbecin producing strain in which mbcM, mbcMT1, mbcMT2, mbcP and mbcP450 have been deleted or inactivated.

50: An engineered strain based on a macbecin producing strain in which mbcM and optionally further post-PKS genes have been deleted or inactivated.

51: An engineered strain based on a macbecin producing strain in which mbcM and optionally further post-PKS genes and/or starter unit biosynthesis genes have been deleted or inactivated.

52: The An engineered strain according to claim 50 in which mbcM has been deleted or inactivated.

53: The engineered strain according to claim 50 in which mbcM, mbcMT1, mbcMT2, mbcP and mbcP450 have been deleted or inactivated.

54: The engineered strain according to claim 50 wherein the macbecin producing strain is A. pretiosum or A. mirum.

55: A method of producing a 18,21-didesoxymacbecin analogue or a pharmaceutically acceptable salt thereof comprising culturing the engineered strain according to claim 50.

56: The method according to claim 55 wherein the 18,21-didesoxymacbecin analogue is defined by formula (I).

57: The composition according to claim 27 further comprising another treatment.

58: The composition according to claim 57 where the other treatment is selected from the group consisting of: methotrexate, leukovorin, prenisone, bleomycin, cyclophosphamide, 5-fluorouracil, paclitaxel, docetaxel, vincristine, vinblastine, vinorelbine, doxorubicin, tamoxifen, toremifene, megestrol acetate, anastrozole, goserelin, anti-HER2 monoclonal antibody (e.g. Herceptin™), capecitabine, raloxifene hydrochloride, EGFR inhibitors, VEGF inhibitors, proteasome inhibitors (e.g. Velcade™), radiotherapy and surgery.

59: The composition according to claim 57 where the other treatment is selected from the group consisting of: bleomycin, capecitabine, cisplatin, cytarabine, cyclophosphamide, doxorubicin, 5-fluorouracil, gemcitabine, leucovorin, methotrexate, mitoxantone, the taxanes including paclitaxel and docetaxel, vincristine, vinblastine and vinorelbine; the hormonal therapies, anastrozole, goserelin, megestrol acetate, prenisone, tamoxifen and toremifene; the monoclonal antibody therapies such as trastuzumab (ani-Her2), cetuximab (ant-EGFR) and bevacizumab (anti-VEGF); and protein kinase inhibitors such as imatinib, dasatinib, gefitinib, erlotinib, lapatinib, temsirolimus; the proteasome inhibitors such as bortezomib; histone deacetylase (HDAC) inhibitors such as vorinostat; angiogenesis inhibitors such as sunitinib, sorafenib, lenalidomide, radiotherapy and surgery.

60: The composition according to claim 57 where the other treatment is selected from the group consisting of conventional chemotherapeutics such as cisplatin, cytarabine, cyclohexylchloroethylnitrosurea, cyclophosphamide, gemcitabine, Ifosfamid, leucovorin, mitomycin, mitoxantone, oxaliplatin and taxanes including taxol and vindesine; hormonal therapies such as anastrozole, goserelin, megestrol acetate and prenisone; monoclonal antibody therapies such as cetuximab (anti-EGFR); protein kinase inhibitors such as dasatinib, lapatinib; histone deacetylase (HDAC) inhibitors such as vorinostat; angiogenesis inhibitors such as sunitinib, sorafenib, lenalidomide; mTOR inhibitors such as temsirolimus; and imatinib.

61: The engineered strain according to claim 51 in which mbcM has been deleted or inactivated.

62: The engineered strain according to claim 51 in which mbcM, mbcMT1, mbcMT2, mbcP and mbcP450 have been deleted or inactivated.

63: The engineered strain according to claim 51 wherein the macbecin producing strain is A. pretiosum or A. mirum.

64: A method of producing a 18,21-didesoxymacbecin analogue or a pharmaceutically acceptable salt thereof comprising culturing the engineered strain according to claim 51.

65: The method according to claim 64 wherein the 18,21-didesoxymacbecin analogue is defined by formula (I).
